(19) United States
(12) Patent Application Publication
(10) Pub. No.: US 2025/0201347 A1
(43) Pub. Date: Jun. 19, 2025

(54) ENCODING FEATURES FOR USE IN MACHINE LEARNING SYSTEMS TO DETECT HEALTH CONDITIONS

(71) Applicant: Flagship Pioneering Innovations VI, LLC, Cambridge, MA (US)

(72) Inventors: Dorna Kashefhaghighi, Menlo Park, CA (US); Amirali Kia, San Diego, CA (US); James Xin Sun, Needham Heights, MA (US); Jocelyn Charlton, London (GB)

(21) Appl. No.: 19/067,342

(22) Filed: Feb. 28, 2025

Related U.S. Application Data
(63) Continuation of application No. PCT/US2023/073069, filed on Aug. 29, 2023.
(60) Provisional application No. 63/373,754, filed on Aug. 29, 2022.

(51) Int. Cl. G16B 40/10

(52) U.S. Cl. CPC G16B 40/10

(57) ABSTRACT

A feature computational module encodes a signal generated by processing a biological sample, given one or more health-condition-informative regions related to an analyte, by using metrics based on marker information occurring within specified windows within a sequence of sites of interest within the health-condition informative regions related to the analyte. Each window has a specified position within a sequence of sites of interest in the health-condition informative region, and a specified size. The size is specified in terms of a number of consecutive sites of interest within the analyte. A metric is thus computed for a plurality of positions within the health-condition informative region. In each metric, a first function of respective marker information for an instance of an analyte for a window is used to compute a respective value for each instance of the analyte in the window. A second function of these respective values is computed to provide one or more values for the one or more metrics for the window.

### BACKGROUND

To detect whether an individual has a health condition, such as cancer, or a characteristic of that health condition, such as a type of cancer or stage of development, a biological sample from the individual typically is processed to generate signals representative of the biological sample. A data processing system determines a likelihood that the individual has a health condition based on data derived from the generated signals. To build the data processing system, techniques from the field of machine learning typically are used. In this context, machine learning techniques are applied to data derived from the signals generated from biological samples. The data derived from the generated signals are commonly referred to as “features”, which are inputs to a computational model. To apply machine learning techniques, biological samples are obtained which originate from individuals for whom a diagnosis for the health condition is known. For each biological sample, respective data corresponding to features used by a computational model are derived. The data for the set of biological samples with a known diagnosis is referred to as a “training set.” The training set includes data both for individuals with the health condition and for individuals without the health condition. A computational model is built, or “trained,” using the training set, and then that trained computational model is applied to data representative of biological samples from individuals with unknown diagnoses to predict whether they likely have the health condition.

### SUMMARY

This Summary introduces a selection of concepts in simplified form that are described further below in the Detailed Description. This Summary neither identifies key or essential features, nor limits the scope, of the claimed subject matter.

To use a computational model in this context, there are several technical problems that arise relating to encoding the signal resulting from processing a biological sample into features.

Some problems arise because the signal includes a large amount of information. One of the challenges involves reducing the volume of data into a set of informative features. However, as the number of features increases, the complexity of the computational model increases. However, as the number of features decreases, information relevant to detection of a health condition may be lost.

Some problems arise because of uncertainty around which metrics and which regions of an analyte are truly informative of a health condition. Omission of some metrics or some regions from the set of features may impact the performance of a trained computational model.

To address such problems, the feature computational module encodes a signal generated by processing a biological sample, given one or more health-condition-informative regions related to an analyte, by using metrics based on marker information occurring within specified windows within a sequence of sites of interest within the health-condition informative regions related to the analyte. Each window has a specified position within a sequence of sites of interest in the health-condition informative region, and a specified size. The size is specified in terms of a number of consecutive sites of interest within the analyte. A metric is thus computed for a plurality of positions within the health-condition informative region.

In each metric, a first function of respective marker information for an instance of an analyte for a window is used to compute a respective value for each instance of the analyte in the window. A second function of these respective values is computed to provide one or more values for the one or more metrics for the window.

An example of a first function applied to an instance of an analyte is a count of occurrences of marker information within the instance of the analyte within the window. The second function first computes counts of the number of instances having each possible count resulting from the first function. The second function then divides the respective number of instances computed for each possible count by the total number of instances, thus providing a fractional value for each of the possible counts for this window.

Another example of a first function applied to an instance of an analyte is a function that identifies a pattern of the marker information in the instance, from among a set of possible patterns, and outputs an indication of that pattern. The second function first computes a count of the number of instances having each possible pattern in a window. The second function then divides the respective number of instances identified for each possible pattern by the total number of instances, thus providing a fractional value for each of the possible patterns for this window.

Using such metrics, each site of interest within each health-condition-informative region can have a plurality of metrics, thus providing numerous metrics for each health-condition-informative region, and substantially increasing the number of features available to a machine learning system. Also, the metrics as described above, lose less information than other metrics, such as metrics that average information over an entire health-condition-informative region. For example, a metric based on a count of patterns preserves a large amount of original information, effectively compressing the original data to reduce storage and computational requirements while preserving information useful to training a machine learning model.

Accordingly, in one aspect, a process encodes a signal generated from processing a biological sample originating from a subject. The signal is indicative of marker information in instances of an analyte in the biological sample. The process involves processing the signal using a computer processor having access to computer storage that stores the signal generated from processing a biological sample originating from a subject. The processing includes computing, for each instance of an analyte in the biological sample, and for each window of a plurality of windows on health-condition-informative regions of the analyte, a respective value for the instance for the window based on a first function of respective marker information for the instance of the analyte for the window. The processing includes computing, for each window of the plurality of windows on the health-condition-informative region, one or more respective metrics for the window based on a second function of the respective values computed for the instances of the analyte for the window based on the first function. The processing includes storing a data structure in memory including the one or more respective metrics computed for the window as a set of values associated with an identifier of the window of the health-condition-informative region. The set of values corresponds to a set of features corresponding to inputs of a computational model. The set of values for the subject for the plurality of windows represents an encoding of the signal from the biological sample of the subject.

In one aspect, a process encodes a signal generated from processing a biological sample originating from a subject. The signal is indicative of marker information in instances of an analyte in the biological sample. The computer-implemented process involves processing the signal using a computer processor having access to computer storage that stores the signal generated from processing a biological sample originating from a subject. The processing includes, for each window of a plurality of windows on a health-condition-informative region of an analyte: computing, for each instance of the analyte overlapping the window, a respective value for the instance for the window based on a first function of the respective marker information for the instance overlapping the window; computing one or more respective metrics for the window based on a second function of the respective values computed for the instances overlapping the window based on the first function; and storing a data structure in memory including the one or more respective metrics computed for the window as a set of values associated with an identifier of the window of the health-condition-informative region. The set of values corresponds to a set of features corresponding to inputs of a computational model. The set of values for the subject for the plurality of windows represents an encoding of the signal generated from processing the biological sample originating from the subject.

In one aspect, a process encodes methylation signals for DNA fragments from a liquid biopsy of a subject, wherein each methylation signal is indicative of methylation of CpGs in a respective DNA fragment. The process involves processing the methylation signals using a computer processor having access to computer storage that stores the methylation signals for the DNA fragments from the liquid biopsy of the subject. The processing includes computing, for each DNA fragment, and for each window of a plurality of windows on a cancer-informative region of DNA of the subject, a respective value for the DNA fragment for the window based on a first function of the respective methylation signal for the DNA fragment for the window. The processing includes computing, for each window of the plurality of windows on the cancer-informative region, one or more respective metrics for the window based on a second function of the respective values computed for the DNA fragments for the window based on the first function. The processing includes storing a data structure in memory including the one or more respective metrics computed for the window as a set of values associated with an identifier of the window of the cancer-informative region. The set of values corresponds to a set of features corresponding to inputs of a computational model. The set of values for the subject for the plurality of windows represents an encoding of the methylation signals for the DNA fragments from the liquid biopsy of the subject.

In one aspect, a process encodes methylation signals for DNA fragments from a liquid biopsy of a subject, wherein each methylation signal is indicative of methylation of CpGs in a respective DNA fragment. The process involves processing the methylation signals using a computer processor having access to computer storage that stores the methylation signals for the DNA fragments from the liquid biopsy of the subject. The processing includes, for each window of a plurality of windows on a cancer-informative region of DNA of the subject: computing, for each DNA fragment overlapping the window, a respective value for the DNA fragment for the window based on a first function of the respective methylation signal for the DNA fragment in the window; computing one or more respective metrics for the window based on a second function of the respective values computed for the DNA fragments for the window based on the first function; storing a data structure in memory including the one or more respective metrics computed for the window as a set of values associated with an identifier of the window of the cancer-informative region. The set of values corresponds to a set of features corresponding to inputs of a computational model. The set of values for the subject for the plurality of windows represents an encoding of the methylation signals for the DNA fragments from the liquid biopsy of the subject.

In one aspect, a process encodes methylation signals for DNA fragments from a liquid biopsy of a subject, wherein each methylation signal is indicative of methylation of CpGs in a respective DNA fragment. The process involves processing the liquid biopsy of the subject to generate in computer storage a respective methylation signal for each of a plurality of DNA fragments in the liquid biopsy, the respective methylation signal indicative of methylation of CpGs in the DNA fragment. The methylations signals are processed using a computer processor having access to the computer storage that stores the methylation signals for the DNA fragments from the liquid biopsy of the subject. The processing includes, for each window of a plurality of windows on a cancer-informative region of DNA of the subject: computing, for each DNA fragment overlapping the window, a respective value for the DNA fragment for the window based on a first function of the respective methylation signal for the DNA fragment in the window; computing one or more respective metrics for the window based on a second function of the respective values computed for the DNA fragments for the window based on the first function; storing a data structure in memory including the one or more respective metrics computed for the window as a set of values associated with an identifier of the window of the cancer-informative region. The set of values corresponds to a set of features corresponding to inputs of a computational model. The set of values for the subject for the plurality of windows represents an encoding of the methylation signals for the DNA fragments from the liquid biopsy of the subject.

In one aspect, a non transitory computer storage medium comprises computer storage with data encoded thereon. The data defines a training set for training a computational model, wherein the data in the training set represents a plurality of processed samples, each processed sample originating from a respective liquid biopsy from a respective subject. The data for each processed sample includes a respective set of values for the processed sample encoding methylation signals from DNA fragments in the processed sample, each set of values including, for each cancer-informative region of DNA, and for each window on the cancer-informative region, one or more respective metrics computed for the window and associated with an identifier of the window of the cancer-informative region, wherein each respective metric comprises a value based on computing a respective value for each DNA fragment for the window based on a first function of the respective methylation signal for the DNA fragment in the window, and a second function of the respective values computed for the DNA fragments for the window based on the first function; and a respective label for the processed sample indicative of a respective known characteristic of the respective subject associated with the processed sample.

In one aspect, a machine includes a processing system comprising at least one computer processor and computer storage, accessible by the processing system. The computer storage includes data defining a training set for training a computational model, data the in the training set representing a plurality of processed samples, each processed sample originating from a respective liquid biopsy from a respective subject, the data for each processed sample including: i. a respective set of values for the processed sample encoding methylation signals from DNA fragments in the processed sample, each set of values including, for each cancer-informative region of DNA, and for each window on the cancer-informative region, one or more respective metrics computed for the window and associated with an identifier of the window of the cancer-informative region, wherein each respective metric comprises a value based on computing a respective value for each DNA fragment for the window based on a first function of the respective methylation signal for the DNA fragment in the window, and a second function of the respective values computed for the DNA fragments for the window based on the first function, and ii. a respective label for the processed sample indicative of a respective known characteristic of the respective subject associated with the processed sample.

For such training, computer program code is stored in the computer storage that when executed by the processing system defines a computational model having inputs for receiving a set of values for a processed sample from the training set, and having an output providing a computed characteristic based on the set of values received at the inputs and parameters of a function, the computer program code further configuring the processing system to access the training set and train the computational model. Training involves repeatedly i. applying the respective sets of values for processed samples in the training set to the inputs of the computational model, ii. receiving, from the output of the computational model, respective outputs in response to the respective set of values applied to the inputs of the computational model, iii. comparing the respective outputs for the respective sets of values to the respective labels for the processed samples corresponding to the input sets of values, and iv. adjusting the parameters of the computational model to reduce error between the respective outputs from the computational model and the respective labels for the processed samples.

In one aspect, a cancer recognition system recognizes a risk of presence of a neoplasm in a subject based on a liquid biopsy from the subject. The system includes equipment having an input that receives a liquid biopsy and an output that provides a methylation signal for the liquid biopsy. The methylation signal is indicative of methylation of CpGs of DNA fragments in the liquid biopsy. The system includes an analytical platform having an input receiving the methylation signal for the liquid biopsy from the equipment and having a processing system that, in response to computer program instructions, is configured to process the methylation signals. To perform such processing, the processing system is configured to, for each window of a plurality of windows on a cancer-informative region of DNA of the subject: compute, for each DNA fragment overlapping the window, a respective value for the DNA fragment for the window based on a first function of the respective methylation signal for the DNA fragment in the window; compute one or more respective metrics for the window based on a second function of the respective values computed for the DNA fragments for the window based on the first function; store a data structure in memory including the one or more respective metrics computed for the window as a set of values associated with an identifier of the window of the cancer-informative region, wherein the set of values corresponds to a set of features corresponding to inputs of a computational model, and wherein the set of values for the subject for the plurality of windows represents an encoding of the methylation signals for the DNA fragments from the liquid biopsy of the subject; and input the computed set of values for the subject for the set of features to a trained computational model that applies a function to the computed set of values to produce an output indicative of a risk of presence of a neoplasm in the subject.

In one aspect, a health condition recognition system recognizes a risk of presence of a health condition in a subject based on a biological sample from the subject. The system includes an analytical platform having an input receiving a signal for the biological sample from the equipment and having a processing system that, in response to computer program instructions, is configured to process the signal. To perform such processing, the processing system is configured to compute, for each instance of an analyte in the biological sample, and for each window of a plurality of windows on health-condition-informative regions of the analyte, a respective value for the instance for the window based on a first function of respective marker information for the instance of the analyte for the window. The processing includes computing, for each window of the plurality of windows on the health-condition-informative region, one or more respective metrics for the window based on a second function of the respective values computed for the instances of the analyte for the window based on the first function. The processing includes storing a data structure in memory including the one or more respective metrics computed for the window as a set of values associated with an identifier of the window of the health-condition-informative region. The set of values corresponds to a set of features corresponding to inputs of a computational model. The set of values for the subject for the plurality of windows represents an encoding of the signal from the biological sample of the subject.

In one aspect, a process detects a risk of presence of a health condition in a subject. The process includes obtaining a biological sample from the subject, and processing the biological sample to obtain a signal indicative of marker information in instances of an analyte in the biological sample. This part of the process can be used to obtain training set samples or samples from individuals for whom status of a health condition is unknown. The signal is processed to provide a set of values for the subject as an encoding of the signal from the biological sample of the subject. The encoded signal is applied to a computational model trained using machine learning techniques to provide an output representing a risk of presence of a health condition in a subject.

In one aspect, a process detects a risk of presence of a neoplasm in a subject. The process includes obtaining a liquid biopsy from the subject, and processing the liquid biopsy to obtain a methylation signal for the liquid biopsy. The part of the process can be used to obtain training set samples or samples from individuals for whom status of a health condition is unknown. The methylation signals are processed to provide a set of values for the subject as an encoding of the methylation signal from the liquid biopsy of the subject. The encoded methylation signal is applied to a computational model trained using machine learning techniques to provide an output representing a risk of presence of a neoplasm in a subject.

In one aspect, a health condition recognition system recognizes a risk of presence of a health condition in a subject based on a biological sample from the subject. The system means for receiving a signal for the biological sample and means for processing the signal. The means for processing computes, for each instance of an analyte in the biological sample, and for each window of a plurality of windows on health-condition-informative regions of the analyte, a respective value for the instance for the window based on a first function of respective marker information for the instance of the analyte for the window. The means for processing computes, for each window of the plurality of windows on the health-condition-informative region, one or more respective metrics for the window based on a second function of the respective values computed for the instances of the analyte for the window based on the first function. The means for processing stores a data structure in memory including the one or more respective metrics computed for the window as a set of values associated with an identifier of the window of the health-condition-informative region. The set of values corresponds to a set of features corresponding to inputs of a computational model. The set of values for the subject for the plurality of windows represents an encoding of the signal from the biological sample of the subject.

In any of the foregoing aspects, the encoded signal represents features that can be applied to a computational model trained using machine learning techniques to provide an output representing a risk of presence of a health condition in a subject.

In any of the foregoing aspects, the respective metrics computed for each of the plurality of windows on the region can be stored in a database as data representing the biological sample or liquid biopsy sample. In some implementations, stored data includes an identifier identifying a liquid biopsy sample or a biological sample. In some implementations, stored data includes an identifier identifying a subject corresponding to the liquid biopsy sample or biological sample. In some implementations, stored data includes a plurality of sets of values corresponding to a plurality of liquid biopsy samples or biological samples from a single subject. In some implementations, the set of values for the subject corresponding to the set of features includes data associating the respective metric for each window for each health-condition-informative region with an identifier of the window and an identifier of the health-condition-informative region. In some implementations, the data is stored in a database allowing search and retrieval of the data given one or more of an identifier of a subject, an identifier of a health-condition-informative region, and identifier of a window, or an identifier of a liquid biopsy sample or biological sample.

In any of the foregoing aspects, samples in a training set can have a label. In some implementations, a label for a sample is selected from a group comprising a label indicative of non-cancer and a label indicative of cancer. In some implementations, a label for a sample is selected from a group comprising a label indicative of a type of cancer. In some implementations, a label for a sample is indicative of a health condition.

In any of the foregoing aspects, processing can further include applying the data representing the biological sample or liquid biopsy sample to a computational model that determines the risk of presence of the early-stage neoplasm in the subject based on the set of values for the set of features.

In any of the foregoing aspects, the first function applied to an instance of an analyte can include a count of occurrences of marker information within the instance of the analyte within the window. In some implementations, the second function of the respective values computed for the instances of the analyte for the window is based on a respective ratio of a count of instances of the analyte having a specific count of occurrences of marker information to a count of instances of analyte for the window.

In any of the foregoing aspects, the first function of the methylation signal for a DNA fragment in a window can include a count of methylated CpGs in the DNA fragment in the window. In some implementations, the second function of the respective values computed for DNA fragments for a window is based on a respective ratio of a count of DNA fragments having a specific count of methylated CpGs to a count of DNA fragments for the window. In some implementations, the second function of the respective values computed for DNA fragments for a window is based on a count of DNA fragments having a specific count of methylated CpGs.

In any of the foregoing aspects, the first function applied to an instance of an analyte can include an indication of a pattern of marker information in the instance in the window, from among a set of possible patterns. In some implementations, the second function of the respective values computed for the instances of the analyte for the window is based on, for each possible pattern of marker information in the window, a ratio of a count of instances of the analyte having the pattern to a count of instances of the analyte in the window.

In any of the foregoing aspects, the first function of the methylation signal can include an indication of a pattern of methylation of CpGs in the DNA fragment in the window. In some implementations, the second function of the respective values computed for DNA fragments for a window is based on, for each possible pattern of methylation in the window, a respective ratio of a count of DNA fragments having the pattern of methylation to a count of the DNA fragments.

In any of the foregoing aspects, a methylation signal can include data indicative of a respective methylation of each CpG in a sequence of CpGs of a DNA fragment. In any of the foregoing aspects, the region can include a plurality of CpGs wherein a number N of CpGs in the region is an integer greater than or equal to 1 and less than or equal to X, a positive integer. In any of the foregoing aspects, the region can include a plurality of CpGs wherein a number N of CpGs in the region is an integer selected from the group consisting of 1, 2, . . . , N.

In any of the foregoing aspects, the first function and the second function can be computed for a plurality of different window sizes for a region. In any of the foregoing aspects, the first function can include, for a window of size W within a region, for each possible pattern of 2 W patterns, a respective count for the pattern, wherein a “count” is when a read has that pattern in that window in that region.

In any of the foregoing aspects, each window has a specified position within a sequence of sites of interest in a health-condition informative region, and a specified size, wherein the size is specified in terms of a number of consecutive sites of interest within the analyte. In any of the foregoing aspects, the health-condition informative regions can be selected from among the genomic regions in one or more of Table I or Table II.

In any of the foregoing aspects, processing a can include includes processing the sample to locate cell-free DNA fragments. In some implementations, the liquid biopsy samples can be obtained and processed such that an average number of cell-free DNA located per cancer-informative region is sufficient to likely include one or more cell-free DNA originating from an early-stage neoplasm if present in the subject. In some implementations, the cell-free DNA fragments originate from health-condition-informative regions of DNA. In some implementations, processing each sample includes processing located cell-free DNA fragments to determine respective methylation information related to CpGs of the located cell-free DNA fragments. In some implementations, the sample is processed such that an average number of cell-free DNA fragments processed per cancer-informative region is sufficient to be likely to detect one or more cell-free DNA fragments per cancer-informative region originating from a present cancer.

In any of the foregoing aspects, a liquid biopsy sample or biological sample comprises plasma obtained from an asymptomatic individual. In any of the foregoing aspects, a liquid biopsy sample or biological sample comprises plasma obtained from a symptomatic individual.

In another aspect, an article of manufacture includes at least one computer storage, and computer program instructions stored on the at least one computer storage. The computer program instructions, when processed by a processing system of a computer, the processing system comprising one or more processing units and storage, configures the computer as set forth in any of the foregoing aspects and/or performs a process as set forth in any of the foregoing aspects.

Any of the foregoing aspects may be embodied as a computer system, as any individual component of such a computer system, as a process performed by such a computer system or any individual component of such a computer system, or as an article of manufacture including computer storage in which computer program instructions are stored and which, when processed by one or more computers, configure the one or more computers to provide such a computer system or any individual component of such a computer system.

The following Detailed Description references the accompanying drawings which form a part this application, and which show, by way of illustration, specific example implementations. Other implementations may be made without departing from the scope of the disclosure.

### BRIEF DESCRIPTION OF THE DRAWINGS

FIG. 1 is a data flow diagram of an example implementation of a system for detecting a health condition.

FIG. 2 is an illustrative example of raw data representing instances of an analyte and markers related to those instances.

FIG. 3A is an illustration of an example implementation of generating features.

FIG. 3B is an illustration of an example implementation of generating features.

FIG. 3C is an illustration of an example implementation of a data structures storing a set of values for a set of features.

FIG. 4A is a flowchart describing steps of an example implementation of generating features.

FIG. 4B is a flowchart describing steps of an example implementation of selecting features.

FIG. 5 is a block diagram of an example general purpose computer.

In the drawings, in the data flow diagrams, a parallelogram indicates an object, e.g., data, which is an input to a component of a system that manipulates the object or an output of such a system, whereas a rectangle indicates the component of the system, e.g., a programmed processor, which manipulates that object.

### DETAILED DESCRIPTION

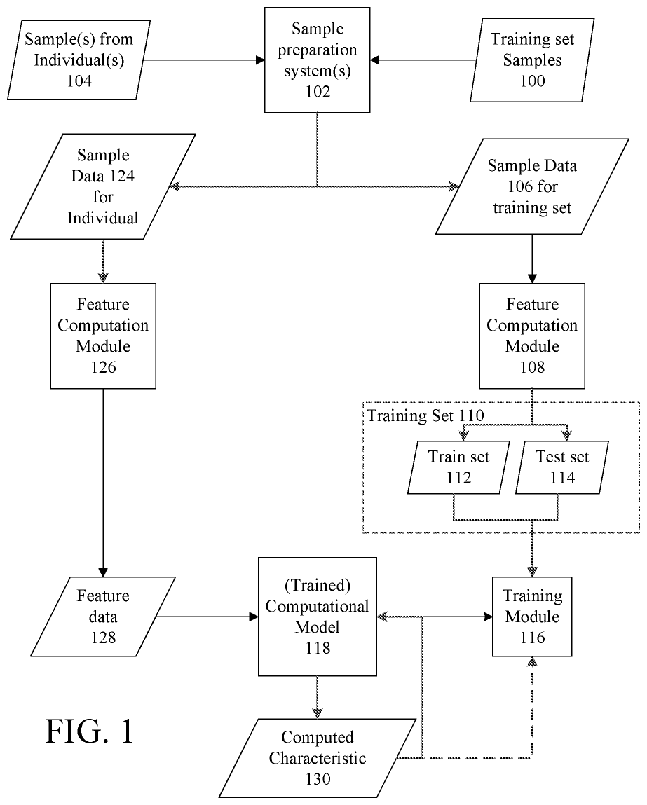

Referring now to the data flow diagram of FIG. 1, an example implementation of a system for detecting a health condition (e.g., cancer) or characteristic of the health condition (e.g., type of cancer or stage of development) using machine learning techniques will be described.

Biological samples from a plurality of individuals with known diagnoses are obtained. These biological samples are used to create data for a training set and therefore are referred to as training set samples 100. The training set samples include biological samples both from individuals with a health condition and from individuals without the health condition. For example, if the health condition is cancer, then the training set samples include biological samples both from individuals with cancer and from individuals without cancer. Machine learning techniques are applied to data derived from signals generated from processing the training set samples to train a computational model (118). The trained computational model 118 is used to detect the health condition in individuals (with unknown diagnoses) by analyzing data derived from signals generated from processing biological samples (104) from those individuals.

The biological samples (whether training set samples 100, or samples from individuals 104) are subjected to several processing steps, represented by sample preparation system(s) 102. Such sample preparation systems generally use reagents, probes, and other ingredients and processes to process a biological sample. Characteristics of the processed biological samples are measured using equipment that generates signals representing these characteristics. Data representing signals are output from the sample preparation system(s) 102. The output of the sample preparation system(s) 102 is called herein sample data, which can be sample data 124 for an individual sample 104, or sample data 106 for a training set sample. Training set sample data 106 and sample data 124 for individuals are derived from such measurements and are output by the sample preparation system. While FIG. 1 illustrates using the same sample preparation system(s) 102 for both training set samples 100 and samples 104 from individuals, it is possible that these biological samples are processed by different sample preparation systems. In some implementations, the additional samples obtained over time are processed by different, lower cost, lower complexity sample preparation systems.

A feature computation module 108 processes the training set sample data 106 to derive respective data for each training set sample. This processing converts the raw data output from the sample preparation system into a set of values for a set of “features” for training and using machine learning models. This set of values for the set of features for a training set sample is stored in association with a respective label indicative of a known characteristic of the individual associated with the training set sample, as training data 110.

The known classification or “label” related to a health condition indicates either the absence of a health condition (or normal) or the presence of the health condition (or abnormal), with terms such as “normal” or “abnormal,” or “healthy” or “unhealthy.” In an implementation where the health condition being detected is cancer, the label can indicate whether, and in some cases what type or to what extent, an individual has cancer, with terms such as non-cancer (or normal) and cancer (or abnormal). Other information about the individual or the biological sample may be known, such as a tissue or liquid of origin, a stage of development, severity of condition, or type of any tumor, or other information.

It should be understood that the labels “normal” and “abnormal,” or “cancer” and “non-cancer,” are not intended to be limiting, and that any label intended to indicate that a biological sample from an individual is not definitively associated with a health condition can be used. A non-exhaustive set of examples of labels includes “normal,” “not detected,” “healthy,” “not identified,” or any other character or set of characters that is interpreted within the computer to discriminate such samples from other samples where the individual has been diagnosed with a health condition or any characteristics of such a health condition.

The training set 110, which comprises sets of values for a set of features which represent the training set samples 100 for which respective classifications or labels for the corresponding individual or biological sample are known, is used by a training module 116 to build or train a computational model 118. Typically, training is performed by dividing the training set into a train set 112 and a test set 114, applying the values for features for the train set samples to the computational model 118, and receiving the computed characteristic 130 from the computational model. The training module 116 repeatedly adjusts parameters of the computational model 118 using the train set 112 while minimizing errors in the computed characteristic 130. The training module 116 then uses the test set 114 to verify how well the computational model 118 has been trained.

The sample data 124 for individual samples are processed by a feature computation module 126 to generate a set of values for the set of features for the individual samples to provide feature data 128. Feature computation module 126 is similar to, if not identical to, feature computation module 108; however, feature data 128 from processed individual samples 124 are inputs to the trained computational model 118, to allow the associated individual to be classified by the trained computational model. Thus, the feature data 128 for processed individual samples does not include a label. The trained computational model 118, when applied to feature data 128, outputs the computed characteristic 130 which indicates the model's determination of the likelihood of the presence of the health condition in the individual.

The computational model 118 can be any computational model for detection of, or prediction of a likelihood of presence of, a health condition in a subject. Examples of such health conditions include, but are not limited to, various neoplasms or tumors, such as pre-cancerous neoplasms, cancer, including early-stage cancerous solid tumors, other diseases, or other disorders, including but not limited to autoimmune diseases, metabolic disorders, neurological disorders, aging, and trauma.

Example types of computational models include, but are not limited to, random forest or other form of classification or decision trees, ensembles of models, and “deep learning” models. Computational models are known by a variety of names, including, but not limited to, classifiers, decision trees, random forests, classification and regression trees, clustering algorithms, predictive models, neural networks, genetic algorithms, deep learning algorithms, convolutional neural networks, artificial intelligence systems, machine learning algorithms, Bayesian models, expert rules, support vector machines, conditional random fields, logistic regression, maximum entropy, among others.

In general, such computational models receive a vector or n-dimensional matrix of features as an input, and provide an output such as a classification, prediction, or other value. The computational model used for classification may or may not be a computational model that is trained by a training set. For example, the computational model can be a simplified set of computations performed on values for the features derived for an individual, where that set of computations is based on insights obtained by analyzing data from the training set. Typically, the computational model computes a function of the features, which may be a linear or non-linear function, to produce an output where the output is indicative of the resulting classification.

The output of the computational model can be a form of prediction, indicating a likelihood that the individual from whom a sample was obtained has a health condition, such as cancer, or a characteristic of a health condition, such as a type of cancer or stage of development. This prediction can be in the form of a probability between zero and one, or a binary output, such as a yes or no answer, or a score (which may be compared to one or more thresholds), or other format. The output can be accompanied by additional information indicating, for example, a level of confidence in the prediction. The output typically depends on the form of the computational model used.

In some implementations for detecting cancer, the output of the computational model indicates the likelihood of presence of cancer, without indicating a type of tumor or cancer, i.e., the affected tissue. In some implementations, the output of the model can indicate a type of cancer, such as its tissue of origin, or stage, or other characteristic, or any combination of these. In some implementations, the output of the model can indicate the presence of cancer, and then one or more additional models can be applied to the data to indicate a type of cancer. In some implementations, a separate model for each type of cancer can be used, and an ensembling process can process the outputs of the separate models.

To apply machine learning techniques to signals generated from biological samples to detect health conditions, one of the problems to solve is the selection of the “features” to be derived from those signals. These features are inputs to a computational model, and challenges arise in how to efficiently discover, compute, store, and otherwise process such features, both for creating and using a training set for training a computational model and for using a trained computational model for classification or prediction. The features to be selected depend in part on the nature of the biological samples and signals generated from processing such samples.

In some implementations, the biological sample is a liquid biopsy, such as a sample of blood or portion of blood such as plasma, urine, stool, saliva, mucous, or other liquid expelled by the body. In some implementations, the biological sample is a tissue biopsy, providing cells extracted from a specific tissue of the body.

A sample preparation system locates an analyte in the biological sample and measures a characteristic of that analyte. As an example, an analyte that can be detected in a biological sample is nucleic acid, for example DNA or RNA. DNA may be whole DNA or cell-free DNA, which are fragments of DNA in the biological sample, for which laboratory equipment such as a sequencer can be used. Such nucleic acids can be processed to measure a variety of different markers at different positions along the nucleic acid. Such markers, can include, but are not limited to, mutations, such as point mutations, (e.g., transition or transversion mutations), deletions, insertions, or other modifications. Such markers can be genetic or epigenetic or of another type.

In some implementations, modifications to DNA can include various types, such as 5-methylcytosine, 5-hydroxymethylcytosine, 5-formylcytosine, 5-carboxylcytosine, or methylation of other DNA bases (e.g., N6-methyladenine), and methylations occurring at different kinds of sites, such as at CpG, CpA, CpT, and CpC sites.

In some implementations, cell-free DNA are processed to determine whether CpGs in the DNA fragment are subject to cytosine methylation. In such an implementation, the sample preparation system includes equipment that generates a signal (herein called a “methylation signal”) for DNA fragments in the sample indicating whether CpGs in the DNA fragments are methylated or not. For example, a modified nucleic acid base (e.g., 5-methylcytosine) may be detected directly using, e.g., nanopore sequencing, or indirectly by chemically converting (e.g., bisulfite converting) the unmodified nucleic acid base to a different chemical entity selectively over the modified nucleic acid base (e.g., cytosine may be converted to uracil using bisulfite conversion under conditions where 5-methylcytosine is not converted to thymine), the presence of which may be detected, e.g., through nucleic acid sequencing.

In such applications, and others, the signal generated from processing a biological sample includes data representing a plurality of instances of an analyte, e.g., a nucleic acid, and respective information about markers related to each instance detected. The respective information about markers related to an analyte generally includes, for a marker, a respective location of the marker on the analyte and information about the marker, such as a state or type or other indication. There is a wide variety of types of sample processing techniques, measurement equipment, analytes, and markers, for different applications that can be used to produce signals representing the instances of an analyte and markers on such analytes within a biological sample.

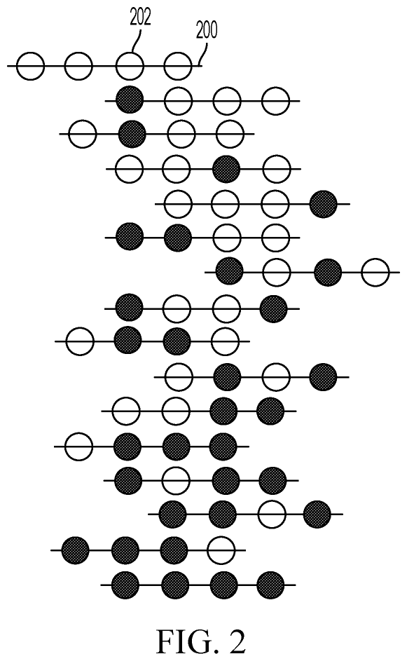

FIG. 2 is an illustrative example of a signal generated by processing a biological sample, which represents instances of an analyte and biomarkers related to those instances. For each instance of an analyte, e.g., a DNA fragment, there is data indicating, for each of a plurality of positions along the instance of the analyte, e.g., distinct CpG sites along a DNA fragment, information about a marker at that position, e.g., whether that CpG is methylated.

Conceptually, using methylation of CpGs in cell-free DNA as an illustrative example, the signal illustrated in FIG. 2 includes a row, e.g., row 200, for each instance of an analyte, such as a single sequenced DNA fragment. Thus, in FIG. 2, data for sixteen instances of an analyte are shown, e.g., sixteen DNA fragments. In FIG. 2, each circle corresponds to a position along the analyte, such as a CpG site. In this example, whether the circle is illustrated as black or white in FIG. 2, is indicative of whether the CpG at that site is methylated (black) or not (white). In some instances, information about a marker at a position in a nucleic acid may not be binary.

The information about the markers for each instance of an analyte in a sample can result in a large amount of data. As an example, in practice, in the case of obtaining methylation state of CpGs in cell-free DNA from a blood sample using deep sequencing, using a DNA sequencer that outputs such data into a FASTQ format data file, the signal generated by processing a single blood sample can be many gigabytes, e.g., 20 to 30 gigabytes, of data.

FIG. 2 also illustrates a relative alignment among the distinct instances of the analyte. In the example of DNA, for example, the position of a DNA fragment within a genome for the individual from which a sample originated can be determined, and each position within the genome can have a respective set of coordinates identifying it. Thus, DNA fragments can be assigned coordinates based on their respective positions within the genome, and then aligned or grouped by those coordinates. Thus, in FIG. 2, column 202 indicates a position on an analyte, such as a single CpG site in a genome, and the distinct instances of the analyte are illustrated as aligned by position on the analyte.

By using the position information for each instance of an analyte, distinct instances of the analyte can be grouped into regions within the analyte. Typically, markers related to health conditions have been found to be localized within identifiable regions of analytes, such as specific genes or regions within the genome in the case of DNA. Thus, the signals generated for each instance of an analyte can be grouped and processed by health-condition-informative regions. Thus, the example in FIG. 2 can be considered to illustrate data about methylation at CpG sites within one informative region of the genome, for multiple DNA fragments obtained from a biological sample. There can be multiple health-condition-informative regions.

Different metrics and health-condition-informative regions if used, may be useful in detecting a variety of diseases or conditions, such as cancer, autoimmune disorders, metabolic disorders, neurological disorders, aging, and trauma. As one example, aspects of the microbiome, such as relative amounts of microorganisms, diversity of microorganisms, gene expression patterns of microorganisms, and others, may be metrics useful in detecting a health condition and may be monitored over time. For instance, sequence information from DNA (e.g., bacterial genomes) or RNA (e.g., 16S rRNA) may be used to distinguish or quantify levels of specific microorganisms in the microbiome.

In certain embodiments, the health-condition-informative regions may be useful for detecting an early-stage health condition, e.g., prior to development of symptoms, such as an early-stage cancer. In certain embodiments, the health-condition-informative regions may be useful for detecting a pre-disease health condition, e.g., a health condition that is not presently a disease state but may later mature to a disease state. Non-limiting examples of pre-disease health conditions include precancer and prediabetes.

The methods described herein may be used in connection with a number of health conditions. The health condition may be cancer or precancer of any solid or liquid tissue. The individual may have hyperplasia, dysplasia, carcinoma in-situ, or a benign tumor. In some embodiments, the tissue associated with a precancer may be gastrointestinal tissue (such as colorectal tissue, pancreatic tissue, gastric tissue, esophageal tissue, hepatocellular tissue, cholangiocellular tissue, oral tissue, lip tissue); urogenital tissue (such as prostate tissue, renal tissue, bladder tissue, penile tissue); gynecological tissue (such as ovarian tissue, cervical tissue, endometrial tissue); lung tissue; head and neck tissue; CNS tissue including glial tissue, astrocytes, retinocyes; breast tissue; skin tissue; thyroid tissue; bone and soft tissue; and hematologic tissue (such as lymphocytes). In some embodiments, the cancer may be acute leukemia, astrocytomas, biliary cancer (cholangiocarcinoma), bone cancer, breast cancer, brain stem glioma, bronchioloalveolar cell lung cancer, cancer of the adrenal gland, cancer of the anal region, cancer of the bladder, cancer of the endocrine system, cancer of the esophagus, cancer of the head or neck, cancer of the kidney, cancer of the parathyroid gland, cancer of the penis, cancer of the pleural/peritoneal membranes, cancer of the salivary gland, cancer of the small intestine, cancer of the thyroid gland, cancer of the ureter, cancer of the urethra, carcinoma of the cervix, carcinoma of the endometrium, carcinoma of the fallopian tubes, carcinoma of the renal pelvis, carcinoma of the vagina, carcinoma of the vulva, cervical cancer, chronic leukemia, colon cancer, colorectal cancer, cutaneous melanoma, ependymoma, epidermoid tumors, Ewing's sarcoma, gastric cancer, glioblastoma, glioblastoma multiforme, glioma, hematologic malignancies, hepatocellular (liver) carcinoma, hepatoma, Hodgkin's Disease, intraocular melanoma, Kaposi sarcoma, lung cancer, lymphomas, medulloblastoma, melanoma, meningioma, mesothelioma, multiple myeloma, muscle cancer, neoplasms of the central nervous system (CNS), neuronal cancer, small cell lung cancer, non-small cell lung cancer, osteosarcoma, ovarian cancer, pancreatic cancer, pediatric malignancies, pituitary adenoma, prostate cancer, rectal cancer, renal cell carcinoma, sarcoma of soft tissue, schwannoma, skin cancer, spinal axis tumors, squamous cell carcinomas, stomach cancer, synovial sarcoma, testicular cancer, uterine cancer, or tumors and their metastases, including refractory versions of any of the above cancers, or any combination thereof.

In some embodiments, the autoimmune disorder may be multiple sclerosis, psoriasis, psoriatic arthritis, rheumatoid arthritis, systemic lupus erythematosus, Crohn's disease, Sjogren's syndrome, Behcet's disease, ulcerative colitis, Guillain-Barre syndrome, or a pre-disease thereof.

In some embodiments, the metabolic disorder may be diabetes, obesity, or a pre-disease thereof.

In some embodiments, the neurological disorder may be Alzheimer's disease, Parkinson's disease, Huntington's disease, amyotrophic lateral sclerosis, or a pre-disease thereof.

In some embodiments, the methods described herein may be informative for treating an individual with an intervention. For example, in some embodiments, a health condition in an individual may be detected as contemplated herein, and an intervention may be provided to the individual to treat the health condition. An individual may be treated using any intervention known to those of ordinary skill in the art. Non-limiting examples of interventions include surgery (e.g., excising diseased or pre-disease tissue from an individual), chemotherapy, gene therapy, gene editing, radiation therapy, or a lifestyle intervention (e.g., change in behavior or habits).

For each training set sample, a respective set of values for the set of features is computed by the feature computation module 108. The specific metrics used, and health-condition-informative regions selected can depend on a variety of factors and may be experimentally determined. The selected metric(s) for the selected health-condition-informative regions are computed for the training set to provide the train set and the test set.

In some implementations, a first set of features is computed for a training set, which can include several candidate features. The candidate features can include one or more candidate metrics, or one or more candidate health-condition-informative regions, or combinations of both. A computational model can be trained using candidate features, and then analyzed to determine which candidate features were more influential in the output of the trained computational model. Such analysis can be used to identify features which are more influential to the model, whether due to the metric or due to the health-condition-informative region. A second set of features can be defined by reducing the first set of features based on those identified features which are more influential, and the trained computational model 118 can be built using the second set of features.

For each individual sample for which a prediction or classification is to be made, a respective set of values for the set of features is computed by the feature computation module 126. The specific metrics used, and health-condition-informative regions selected can depend on a variety of factors and may be experimentally determined. The selected metric(s) for the selected health-condition-informative regions are computed for the sample, and then input to the trained computation model 118.

In some implementations, related to identifying individually informative CpGs, such CpGs typically are found in regions of the human genome referred to as “CGI”'s. Several CGI's may include individually informative CpGs. In some implementations the system may consider specifically information related to a set of CGIs known to be informative of cancer or other health conditions. In some of these implementations, a sample of cell free DNA is processed to obtain a first set of methylation data by measuring methylation level at a plurality of CpGs within one or more genomic regions set forth in one or more of Table I or Table II, attached as appendices to this Specification. These tables, as listed below, referred to in this Specification and Claims, form a part of this Specification and are hereby incorporated by reference into this Specification.

Table I—Listing of CGIs identified in U.S. Patent Publication 2020/0109456A1, which is hereby incorporated by reference, specifically the “Table I” of CGIs listed in that published patent application.

Table II—Listing of CGIs identified in PCT Patent Publication WO2022/133315, which is hereby incorporated by reference, specifically “Table 2” and “Table 3” of CGIs listed in that published patent application.

To use a computational model in this context, there are several technical problems that arise relating to encoding the signal resulting from processing a biological sample into features.

Some problems arise because the signal includes a large amount of information. One of the challenges involves reducing the volume of data into a set of informative features. However, as the number of features increases, the complexity of the computational model increases. However, as the number of features decreases, information relevant to detection of a health condition may be lost.

Some problems arise because of uncertainty around which metrics and which regions of an analyte are truly informative of a health condition. Omission of some metrics or some regions from the set of features may impact the performance of a trained computational model.

To address such problems, the feature computational module encodes a signal generated by processing a biological sample, given one or more health-condition-informative regions related to an analyte, by using metrics based on marker information occurring within a plurality of distinct windows within health-condition informative regions related to the analyte. Each window has a specified position within a sequence of sites of interest in a health-condition informative region, and a specified size. The size is specified in terms of a number of consecutive sites of interest within the analyte. A metric is thus computed for a plurality of positions within the health-condition informative region.

In some implementations, the feature computation module begins encoding by processing each instance of the analyte. For example, the feature computation module computes, for each instance of an analyte in the biological sample, and for each window of a plurality of windows on health-condition-informative regions of the analyte, a respective value for the instance for the window based on a first function of respective marker information for the instance of the analyte for the window. After processing instances of the analyte, the feature computation module then computes, for each window of the plurality of windows on the health-condition-informative region, one or more respective metrics for the window based on a second function of the respective values computed for the instances of the analyte for the window.

In some implementations, the feature computation module begins encoding by processing each window within a health-condition-informative region. For example, the feature computational model computes, for each window of a plurality of windows on a health-condition-informative region of the analyte, for each instance of the analyte overlapping the window, a respective value for the instance of the analyte for the window based on a first function of the respective marker information for the instance overlapping the window. After computing the respective values for the instances of the analyte that overlap the window, the feature computation module then computes one or more respective metrics for the window based on a second function of the respective values computed for the instance overlapping the window.

Example implementations of the feature computation module 108 will now be described. To simplify the following description, in the example described herein, training set sample data 106 and sample data 124 for individuals are described as having been derived from a methylation signal output by the sample preparation system 102 (See FIG. 1).

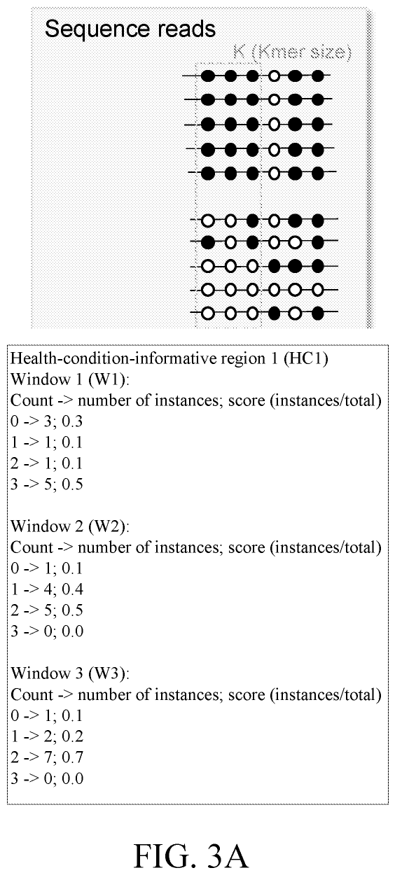

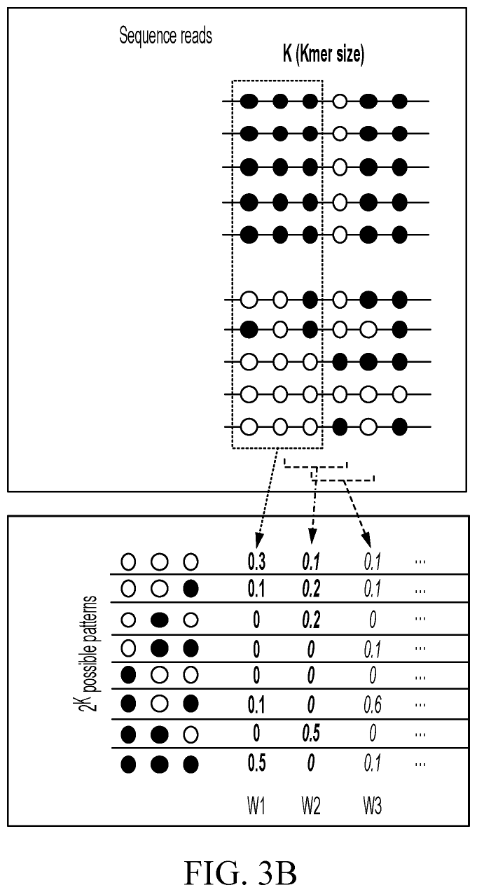

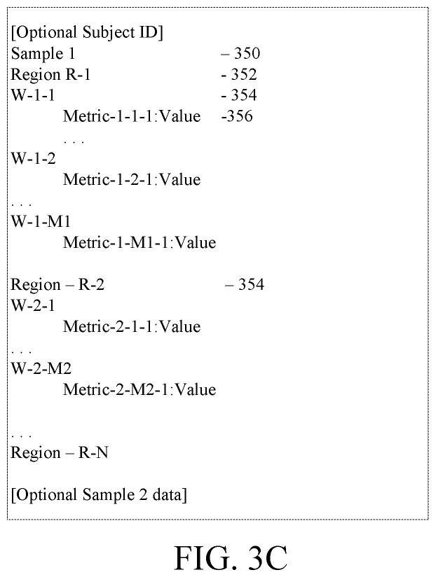

In FIGS. 3A and 3B, example, illustrative marker information for instances of an analyte is shown schematically for the purposes of simplifying this explanation. In this example, there are ten (10) instances of an analyte, each having a length of six (6) sites of interest, at which marker information is a binary value, indicated by a black or white circle. Each window has a size of three (3) consecutive sites of interest within the analyte. There are four (4) windows of size three (3) (i.e., a first window that includes the first, second, and third sites of interest from the left, a second window that includes the second, third, and fourth sites of interest from the left, a third window that includes the third, fourth, and fifth sites of interest from the left, and a fourth window that includes the fourth, fifth, and sixth sites of interest from the left), but computations for three (3) windows are shown.

In FIG. 3A an example of a first function applied to an instance of an analyte is a count of occurrences of marker information within the instance of the analyte within the window. For example, where the marker information is methylation of a CpG site, this function can be a count of methylated CpGs in the window. That is, if the window has a size of three sites of interest, then there are four possible counts: 0, 1, 2, and 3. Note that inverse results would be obtained if the count was of unmethylated CpGs in the window, but such results when used in training would have the same effect.

In FIG. 3A, the second function first computes counts of the number of instances having each possible count resulting from the first function. That is, if the window has a size of three sites of interest, for which there are four possible counts (0, 1, 2, and 3), for that window the second function first computes a count of the number of instances with a count of zero, a count of the number of instances with a count of one, a count of the number of instances with a count of two, and a count of the number of instances with a count of three. The second function then divides the respective number of instances computed for each possible count by the total number of instances, thus providing a fractional value for each of the possible counts for this window.

In this example in FIG. 3A, for this health-condition-informative region (say, “HC1”), there are windows “W1”, “W2”, and “W3”, each of which has four (4) values, representing the respective count for each possible count of methylated CpGs among the instances that overlap that window. Because there are ten (10) instances, each of these values is divided by 10 in the second function, to provide the respective final four output values for each window.

In FIG. 3B an example of a first function applied to an instance of an analyte is a function that identifies a pattern of the marker information in the instance, from among a set of possible patterns, and outputs an indication of that pattern. A pattern is a unique sequence of marker information along the sites of interest in a window. For example, if the window has a size of three sites of interest, and if the marker information for the sites of information is binary, then there are eight possible patterns. For example, where the marker information is methylation of a CpG site, each possible pattern of methylation in a window is a distinct sequence of the methylation state (e.g., methylated or unmethylated) of the CpG sites along the sequence of consecutive CpG sites in the window. When the marker information is methylation of CpG sites, this first function, applied to an instance of a DNA fragment in a window, outputs an indication of which of the possible patterns of methylation of CpGs is present in the window in that DNA fragment.

The second function first computes a count of the number of instances having each possible pattern in a window. That is, for that window, the second function first produces a count of the number of instances with the first pattern, a count of the number of instances with the second pattern, and so on. The second function then divides the respective number of instances identified for each possible pattern by the total number of instances, thus providing a fractional value for each of the possible patterns for this window, as shown in the bottom panel of FIG. 3B.

In this example in FIG. 3B, for this health-condition-informative region (say, “HC1”), there are windows “W1”, “W2”, and “W3”, each of which has eight values, representing the respective number of occurrences each possible pattern among the instances that overlap that window divided by the number of instances, in this case ten (10).

In any of the foregoing example implementations, and in other implementations, a size of a health-condition-informative region, in terms of a number of sites of interest within an instance of an analyte, can vary. For example, cancer-informative regions of DNA may be as small as a single CpG site, and may include several 10's, 100's, or 1000's of CpG sites. Within a set of features, there may be a plurality of health-condition-informative regions, each having its own respective size.

In any of the foregoing example implementations, and in other implementations, a size of a window in a health-condition-informative region, in terms of a number of sites of interest within an instance of an analyte, can vary. Generally, the number of sites of interest is a positive integer number that ranges between 1 and N. In some example implementations, N is less than or equal to 10, or 9, or 8, or 7, or 6, or 5, or 4, or 3. Within a set of features, there may be a plurality of health-condition-informative regions, each having its own respective window size or set of window sizes. Different window sizes may be used in different regions. The same window size may be used in different regions. A region may have metrics computed for it for multiple different window sizes. Windows may be over-lapping or non-overlapping.

The computed sets of values for the set of features for samples can be stored in a data structure, which can be stored in a database, memory, or other computer storage for use in connection with the computational model, or for other purposes.

In some implementations, the sets of values for the set of features for a sample can be stored in association with an identifier of the subject, or an identifier of the sample, or both, so that the identifier of the subject or the identifier of the sample, or both, can be used to access the set of values from the computer storage. In some implementations, each computed value can be associated with an identifier of the cancer-informative region, and an identifier of the window within that region, to which the value corresponds.

Accordingly, an example implementation of such a data structure is shown in FIG. 3C. A set of values for a set of features is stored for a biological sample originating from a subject. The data structure can include an optional identifier for the subject, and an optional identifier for the biological sample. The latter identifier is useful when there are multiple samples for a single subject. For a sample, as indicated at 350, the set of features includes one or more metrics, for each of one or more windows 354, e.g., window “W-1-1”, within each of one or more health-condition-informative regions 352, e.g., region “R1”. For each feature, e.g., R-1, W-1-1, Metric, the computed value, e.g., Value 356, is stored. The number of windows in each region can be different for each region. The size of the window can be different for each window. The metric(s) computed for the window can be different for each window.

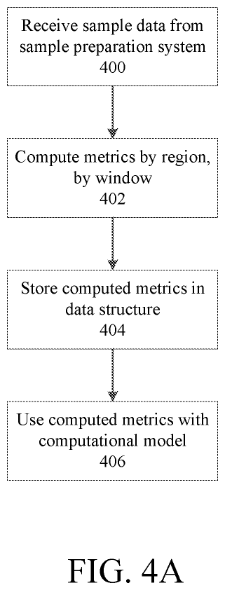

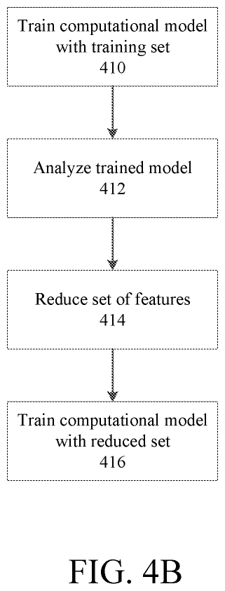

A flowchart for an example process for encoding a signal from a sample is shown in FIG. 4A. Sample data is received 400 from a sample preparation system. Given a type of analyte and health condition, the feature computation module computes 402, for a health-condition-informative region, metrics for each window with the region based on the instances of the analyte found in the sample. These computed metrics can be stored 404 in a data structure, and then used 406 with a computational model, whether stored permanently in computer storage as part of a training set, or in memory or other data structure for later processing.

A flowchart for an example process for selecting features based on such an encoding is shown in FIG. 4B. A first set of features is computed for a training set, which can include several candidate features. The candidate features can include one or more candidate metrics, or one or more candidate health-condition-informative regions, or combinations of both. The computational model is trained 410 using candidate features from this first set. The trained model is analyzed 412 to determine which candidate features were more influential in the output of the trained computational model. Such analysis can be used to identify features which are more influential to the model, whether due to the metric or due to the health-condition-informative region. A second set of features can be defined by reducing 414 the first set of features based on those identified features which are more influential. A trained computational model (e.g., 118 in FIG. 1) can be built by training using the second set of features.

The foregoing description provides example implementations of a computer system implementing these techniques. The various computers used in this computer system can be implemented using one or more general-purpose computers, such as client devices including mobile devices and client computers, one or more server computers, or one or more database computers, or combinations of any two or more of these, which can be programmed to implement the functionality such as described in the example implementations.

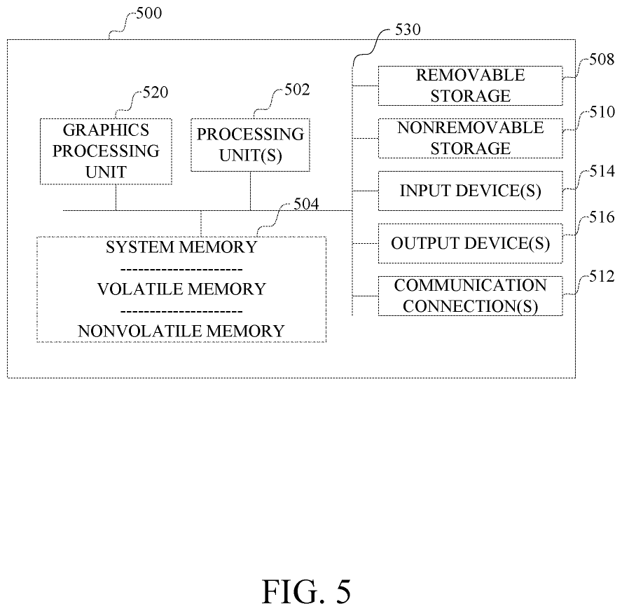

FIG. 5 is a block diagram of a general-purpose computer which processes computer programs using a processing system. Computer programs on a general-purpose computer generally include an operating system and applications. The operating system is a computer program running on the computer that manages access to resources of the computer by the applications and the operating system. The resources generally include memory, storage, communication interfaces, input devices and output devices.

Examples of such general-purpose computers include, but are not limited to, larger computer systems such as server computers, database computers, desktop computers, laptop and notebook computers, as well as mobile or handheld computing devices, such as a tablet computer, handheld computer, smart phone, media player, personal data assistant, audio and/or video recorder, or wearable computing device.

With reference to FIG. 5, an example computer 500 comprises a processing system including at least one processing unit 502 and a memory 504. The computer can have multiple processing units 502 and multiple devices implementing the memory 504. A processing unit 502 can include one or more processing cores (not shown) that operate independently of each other. Additional co-processing units, such as graphics processing unit 520, also can be present in the computer. The memory 504 may include volatile devices (such as dynamic random-access memory (DRAM) or other random-access memory device), and non-volatile devices (such as a read-only memory, flash memory, and the like) or some combination of the two, and optionally including any memory available in a processing device. Other memory such as dedicated memory or registers also can reside in a processing unit. Such a memory configures is delineated by the dashed line 504 in FIG. 5. The computer 500 may include additional storage (removable and/or non-removable) including, but not limited to, solid state devices, or magnetically recorded or optically recorded disks or tape. Such additional storage is illustrated in FIG. 5 by removable storage 508 and non-removable storage 510. The various components in FIG. 5 are interconnected by an interconnection mechanism, such as one or more buses 530.

A computer storage medium is any medium in which data can be stored in and retrieved from addressable physical storage locations by the computer. Computer storage media includes volatile and nonvolatile memory devices, and removable and non-removable storage devices. Memory 504, removable storage 508, and non-removable storage 510 are all examples of computer storage media. Some examples of computer storage media are RAM, ROM, EEPROM, flash memory or other memory technology, CD-ROM, digital versatile disks (DVD) or other optically or magneto-optically recorded storage device, magnetic cassettes, magnetic tape, magnetic disk storage or other magnetic storage devices. Computer storage media and communication media are mutually exclusive categories of media.

The computer 500 may also include communications connection(s) 512 that allow the computer to communicate with other devices over a communication medium. Communication media typically transmit computer program code, data structures, program modules or other data over a wired or wireless substance by propagating a modulated data signal such as a carrier wave or other transport mechanism over the substance. The term “modulated data signal” means a signal that has one or more of its characteristics set or changed in such a manner as to encode information in the signal, thereby changing the configuration or state of the receiving device of the signal. By way of example, and not limitation, communication media includes wired media such as a wired network or direct-wired connection, and wireless media include any non-wired communication media that allows propagation of signals, such as acoustic, electromagnetic, electrical, optical, infrared, radio frequency and other signals. Communications connections 512 are devices, such as a network interface or radio transmitter, that interface with the communication media to transmit data over and receive data from signals propagated through communication media.

The communications connections can include one or more radio transmitters for telephonic communications over cellular telephone networks, and/or a wireless communication interface for wireless connection to a computer network. For example, a cellular connection, a Wi-Fi connection, a Bluetooth connection, and other connections may be present in the computer. Such connections support communication with other devices, such as to support voice or data communications.

The computer 500 may have various input device(s) 514 such as a various pointer (whether single pointer or multi-pointer) devices, such as a mouse, tablet and pen, touchpad and other touch-based input devices, stylus, image input devices, such as still and motion cameras, audio input devices, such as a microphone. The computer may have various output device(s) 516 such as a display, speakers, printers, and so on, also may be included. These devices are well known in the art and need not be discussed at length here.

The various storage 510, communication connections 512, output devices 516 and input devices 514 can be integrated within a housing of the computer or can be connected through various input/output interface devices on the computer, in which case the reference numbers 510, 512, 514 and 516 can indicate either the interface for connection to a device or the device itself as the case may be.

An operating system of the computer typically includes computer programs, commonly called drivers, which manage access to the various storage 510, communication connections 512, output devices 516 and input devices 514. Such access generally includes managing inputs from and outputs to these devices. In the case of communication connections, the operating system also may include one or more computer programs for implementing communication protocols used to communicate information between computers and devices through the communication connections 512.

Any of the foregoing aspects may be embodied as a computer system, as any individual component of such a computer system, as a process performed by such a computer system or any individual component of such a computer system, or as an article of manufacture including computer storage in which computer program code is stored and which, when processed by the processing system(s) of one or more computers, configures the processing system(s) of the one or more computers to provide such a computer system or individual component of such a computer system.

Each component (which also may be called a “module” or “engine” or “computational model” or the like), of a computer system such as described herein, and which operates on one or more computers, can be implemented as computer program code processed by the processing system(s) of one or more computers. Computer program code includes computer-executable instructions and/or computer-interpreted instructions, such as program modules, which instructions are processed by a processing system of a computer. Such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

It should be understood that the subject matter defined in the appended claims is not necessarily limited to the specific implementations described above. The specific implementations described above are disclosed as examples only.

What is claimed is:

TABLE I
Characterization of genomic location of the feature, the number of
cancer types as reported in the disclosure in which they are specifically
methylated, and one of two additional designations - their orthology
to regions that are methylated during mouse development (Mouse ExE
ortholog) or their specific methylation in human placenta and at
least 8 of the 15 human cancer types examined.

| Human CGI (hg19) | # Cancers | Group |
|---|---|---|
| chr1: 1181756-1182470 | 3 | Mouse ExE ortholog |
| chr1: 1470604-1471450 | 0 | Mouse ExE ortholog |
| chr1: 2772126-2772665 | 1 | Mouse ExE ortholog |
| chr1: 4713989-4716555 | 9 | Mouse ExE ortholog |
| chr1: 18436551-18437673 | 5 | Mouse ExE ortholog |
| chr1: 18956895-18959829 | 12 | Mouse ExE ortholog |
| chr1: 18962842-18963481 | 7 | Mouse ExE ortholog |
| chr1: 18967251-18968119 | 0 | Mouse ExE ortholog |
| chr1: 19203874-19204234 | 1 | Mouse ExE ortholog |
| chr1: 21616380-21617101 | 0 | Mouse ExE ortholog |
| chr1: 25255527-25259005 | 5 | Mouse ExE ortholog |
| chr1: 29585897-29586598 | 1 | Mouse ExE ortholog |
| chr1: 34628783-34630976 | 7 | Mouse ExE ortholog |
| chr1: 39980365-39981768 | 13 | Mouse ExE ortholog |
| chr1: 40235767-40237190 | 0 | Mouse ExE ortholog |
| chr1: 41831976-41832542 | 0 | Mouse ExE ortholog |
| chr1: 46951168-46951792 | 12 | Mouse ExE ortholog |
| chr1: 47909712-47911020 | 13 | Mouse ExE ortholog |
| chr1: 53742297-53742845 | 2 | Mouse ExE ortholog |
| chr1: 55505060-55506015 | 3 | Mouse ExE ortholog |
| chr1: 61515875-61516831 | 1 | Mouse ExE ortholog |
| chr1: 63782394-63790471 | 2 | Mouse ExE ortholog |
| chr1: 65731411-65731849 | 7 | Mouse ExE ortholog |
| chr1: 66258440-66258918 | 12 | Mouse ExE ortholog |
| chr1: 77747314-77748224 | 7 | Mouse ExE ortholog |
| chr1: 91172102-91172771 | 10 | Mouse ExE ortholog |
| chr1: 91176404-91176701 | 14 | Mouse ExE ortholog |
| chr1: 92945907-92952609 | 1 | Mouse ExE ortholog |
| chr1: 115880167-115881332 | 6 | Mouse ExE ortholog |
| chr1: 116380359-116382364 | 3 | Mouse ExE ortholog |
| chr1: 156105707-156106171 | 5 | Mouse ExE ortholog |
| chr1: 156338758-156339251 | 0 | Mouse ExE ortholog |
| chr1: 156358050-156358252 | 10 | Mouse ExE ortholog |
| chr1: 156390403-156391581 | 1 | Mouse ExE ortholog |
| chr1: 160340604-160340843 | 13 | Mouse ExE ortholog |
| chr1: 161695637-161697298 | 0 | Mouse ExE ortholog |
| chr1: 177133392-177133846 | 10 | Mouse ExE ortholog |
| chr1: 180198119-180204975 | 0 | Mouse ExE ortholog |
| chr1: 197887088-197887791 | 8 | Mouse ExE ortholog |
| chr1: 201252452-201253648 | 3 | Mouse ExE ortholog |
| chr1: 202678881-202679769 | 6 | Mouse ExE ortholog |
| chr1: 214156000-214156851 | 10 | Mouse ExE ortholog |
| chr1: 214158726-214159080 | 9 | Mouse ExE ortholog |
| chr1: 221057463-221057757 | 11 | Mouse ExE ortholog |
| chr1: 221067447-221068185 | 12 | Mouse ExE ortholog |
| chr1: 226075150-226075680 | 12 | Mouse ExE ortholog |
| chr1: 248020330-248021252 | 5 | Mouse ExE ortholog |
| chr10: 50602989-50606783 | 8 | Mouse ExE ortholog |
| chr10: 50817601-50820356 | 11 | Mouse ExE ortholog |
| chr10: 71331926-71333392 | 7 | Mouse ExE ortholog |
| chr10: 88122924-88127364 | 5 | Mouse ExE ortholog |
| chr10: 94820026-94823252 | 11 | Mouse ExE ortholog |
| chr10: 101279941-101280382 | 6 | Mouse ExE ortholog |
| chr10: 101281181-101282116 | 8 | Mouse ExE ortholog |
| chr10: 102419147-102419668 | 11 | Mouse ExE ortholog |
| chr10: 102473206-102474026 | 9 | Mouse ExE ortholog |
| chr10: 102484200-102484476 | 11 | Mouse ExE ortholog |
| chr10: 102489343-102491011 | 2 | Mouse ExE ortholog |
| chr10: 102507482-102509646 | 4 | Mouse ExE ortholog |
| chr10: 102893660-102895059 | 13 | Mouse ExE ortholog |
| chr10: 102896342-102896665 | 13 | Mouse ExE ortholog |
| chr10: 102899822-102900263 | 13 | Mouse ExE ortholog |
| chr10: 102975969-102978096 | 4 | Mouse ExE ortholog |
| chr10: 105361784-105362188 | 0 | Mouse ExE ortholog |
| chr10: 105420685-105421076 | 6 | Mouse ExE ortholog |
| chr10: 106399567-106402812 | 13 | Mouse ExE ortholog |
| chr10: 118899247-118900329 | 14 | Mouse ExE ortholog |
| chr10: 119000435-119001530 | 7 | Mouse ExE ortholog |
| chr10: 119311204-119312104 | 10 | Mouse ExE ortholog |
| chr10: 119312766-119313563 | 5 | Mouse ExE ortholog |
| chr10: 124905634-124906161 | 12 | Mouse ExE ortholog |
| chr10: 124907283-124911035 | 11 | Mouse ExE ortholog |
| chr10: 129534410-129537366 | 10 | Mouse ExE ortholog |
| chr11: 725596-726870 | 6 | Mouse ExE ortholog |
| chr11: 8190226-8190671 | 10 | Mouse ExE ortholog |
| chr11: 17740789-17743779 | 7 | Mouse ExE ortholog |
| chr11: 20181200-20182325 | 14 | Mouse ExE ortholog |
| chr11: 20622720-20623399 | 13 | Mouse ExE ortholog |
| chr11: 31825743-31826967 | 14 | Mouse ExE ortholog |
| chr11: 31839363-31839813 | 14 | Mouse ExE ortholog |
| chr11: 31848487-31848776 | 14 | Mouse ExE ortholog |
| chr11: 32452144-32452708 | 13 | Mouse ExE ortholog |
| chr11: 32454874-32457311 | 9 | Mouse ExE ortholog |
| chr11: 36397926-36399398 | 0 | Mouse ExE ortholog |
| chr11: 44327240-44327932 | 2 | Mouse ExE ortholog |
| chr11: 46299544-46300216 | 0 | Mouse ExE ortholog |
| chr11: 46366876-46367101 | 3 | Mouse ExE ortholog |
| chr11: 64136814-64138187 | 0 | Mouse ExE ortholog |
| chr11: 65352231-65353134 | 13 | Mouse ExE ortholog |
| chr11: 69517840-69519929 | 0 | Mouse ExE ortholog |
| chr11: 69831571-69832484 | 1 | Mouse ExE ortholog |
| chr11: 70672834-70673055 | 8 | Mouse ExE ortholog |
| chr11: 72532612-72533774 | 2 | Mouse ExE ortholog |
| chr11: 79148358-79152200 | 4 | Mouse ExE ortholog |
| chr11: 124629723-124629926 | 6 | Mouse ExE ortholog |
| chr12: 3475010-3475654 | 3 | Mouse ExE ortholog |
| chr12: 5018585-5021171 | 9 | Mouse ExE ortholog |
| chr12: 6438272-6438931 | 2 | Mouse ExE ortholog |
| chr12: 15475318-15475901 | 8 | Mouse ExE ortholog |
| chr12: 29302034-29302954 | 2 | Mouse ExE ortholog |
| chr12: 45444202-45445386 | 8 | Mouse ExE ortholog |
| chr12: 49183049-49183282 | 0 | Mouse ExE ortholog |
| chr12: 49371690-49375550 | 1 | Mouse ExE ortholog |
| chr12: 49484920-49485178 | 7 | Mouse ExE ortholog |
| chr12: 53491572-53491955 | 0 | Mouse ExE ortholog |
| chr12: 54338761-54339168 | 11 | Mouse ExE ortholog |
| chr12: 54366815-54369103 | 2 | Mouse ExE ortholog |
| chr12: 54378696-54380102 | 1 | Mouse ExE ortholog |
| chr12: 54423427-54423712 | 11 | Mouse ExE ortholog |
| chr12: 54440642-54441543 | 9 | Mouse ExE ortholog |
| chr12: 54447744-54448091 | 10 | Mouse ExE ortholog |
| chr12: 54519768-54520457 | 2 | Mouse ExE ortholog |
| chr12: 57618769-57619402 | 4 | Mouse ExE ortholog |
| chr12: 58003880-58004249 | 7 | Mouse ExE ortholog |
| chr12: 58158855-58160000 | 0 | Mouse ExE ortholog |
| chr12: 63543636-63544967 | 12 | Mouse ExE ortholog |
| chr12: 75602991-75603344 | 13 | Mouse ExE ortholog |
| chr12: 99139386-99139769 | 10 | Mouse ExE ortholog |
| chr12: 101109863-101111622 | 11 | Mouse ExE ortholog |
| chr12: 106979429-106981086 | 12 | Mouse ExE ortholog |
| chr12: 113590806-113591304 | 0 | Mouse ExE ortholog |
| chr12: 113900750-113906442 | 5 | Mouse ExE ortholog |
| chr12: 113908887-113910681 | 9 | Mouse ExE ortholog |
| chr12: 113913615-113914322 | 13 | Mouse ExE ortholog |
| chr12: 114878143-114879155 | 12 | Mouse ExE ortholog |
| chr12: 114886354-114886579 | 11 | Mouse ExE ortholog |
| chr12: 115109503-115110061 | 7 | Mouse ExE ortholog |
| chr12: 117798076-117799448 | 9 | Mouse ExE ortholog |
| chr12: 120835586-120835927 | 11 | Mouse ExE ortholog |
| chr12: 122016170-122017693 | 4 | Mouse ExE ortholog |
| chr12: 130387609-130389139 | 13 | Mouse ExE ortholog |
| chr12: 130908777-130909191 | 0 | Mouse ExE ortholog |
| chr13: 27334226-27335205 | 8 | Mouse ExE ortholog |
| chr13: 28498226-28499046 | 14 | Mouse ExE ortholog |
| chr13: 36049570-36050159 | 8 | Mouse ExE ortholog |
| chr13: 36052553-36053119 | 7 | Mouse ExE ortholog |
| chr13: 79182859-79183880 | 2 | Mouse ExE ortholog |
| chr13: 84453664-84453897 | 14 | Mouse ExE ortholog |
| chr13: 108518334-108518633 | 9 | Mouse ExE ortholog |
| chr13: 109147798-109149019 | 13 | Mouse ExE ortholog |
| chr14: 36974548-36975425 | 9 | Mouse ExE ortholog |
| chr14: 36986362-36990576 | 4 | Mouse ExE ortholog |
| chr14: 37049333-37051726 | 6 | Mouse ExE ortholog |
| chr14: 37116188-37117628 | 1 | Mouse ExE ortholog |
| chr14: 38678245-38680937 | 7 | Mouse ExE ortholog |
| chr14: 54418677-54418881 | 9 | Mouse ExE ortholog |
| chr14: 57274607-57276840 | 13 | Mouse ExE ortholog |
| chr14: 57283967-57284558 | 11 | Mouse ExE ortholog |
| chr14: 69256676-69257036 | 5 | Mouse ExE ortholog |
| chr14: 74706188-74708192 | 11 | Mouse ExE ortholog |
| chr14: 95237622-95238211 | 13 | Mouse ExE ortholog |
| chr14: 105167663-105168129 | 0 | Mouse ExE ortholog |
| chr15: 33009530-33011696 | 7 | Mouse ExE ortholog |
| chr15: 40268581-40269061 | 6 | Mouse ExE ortholog |
| chr15: 45408573-45409528 | 7 | Mouse ExE ortholog |
| chr15: 47476369-47477499 | 10 | Mouse ExE ortholog |
| chr15: 49254984-49255564 | 1 | Mouse ExE ortholog |
| chr15: 60287107-60287663 | 8 | Mouse ExE ortholog |
| chr15: 60296135-60298520 | 5 | Mouse ExE ortholog |
| chr15: 67073306-67073943 | 0 | Mouse ExE ortholog |
| chr15: 74419870-74423044 | 3 | Mouse ExE ortholog |
| chr15: 79724099-79725643 | 6 | Mouse ExE ortholog |
| chr15: 89914363-89915061 | 12 | Mouse ExE ortholog |
| chr15: 89920793-89922768 | 7 | Mouse ExE ortholog |
| chr15: 89949373-89951130 | 12 | Mouse ExE ortholog |
| chr15: 91642908-91643702 | 2 | Mouse ExE ortholog |
| chr15: 96873408-96877721 | 2 | Mouse ExE ortholog |
| chr16: 2228190-2230946 | 0 | Mouse ExE ortholog |
| chr16: 3013016-3013228 | 3 | Mouse ExE ortholog |
| chr16: 3190765-3191389 | 0 | Mouse ExE ortholog |
| chr16: 22824616-22826459 | 12 | Mouse ExE ortholog |
| chr16: 48844551-48845264 | 13 | Mouse ExE ortholog |
| chr16: 49311413-49312308 | 11 | Mouse ExE ortholog |
| chr16: 49314037-49316543 | 9 | Mouse ExE ortholog |
| chr16: 49872449-49872926 | 11 | Mouse ExE ortholog |
| chr16: 51147490-51147944 | 8 | Mouse ExE ortholog |
| chr16: 51168266-51169110 | 13 | Mouse ExE ortholog |
| chr16: 54970301-54972846 | 7 | Mouse ExE ortholog |
| chr16: 55513220-55513526 | 7 | Mouse ExE ortholog |
| chr16: 58030214-58031633 | 0 | Mouse ExE ortholog |
| chr16: 62069121-62070634 | 11 | Mouse ExE ortholog |
| chr16: 67208067-67208678 | 0 | Mouse ExE ortholog |
| chr16: 67571252-67572728 | 1 | Mouse ExE ortholog |
| chr16: 68480864-68482822 | 1 | Mouse ExE ortholog |
| chr16: 86530747-86532994 | 7 | Mouse ExE ortholog |
| chr16: 86549069-86550512 | 6 | Mouse ExE ortholog |
| chr16: 86612188-86613821 | 8 | Mouse ExE ortholog |
| chr16: 88943427-88943669 | 0 | Mouse ExE ortholog |
| chr17: 12568667-12569335 | 3 | Mouse ExE ortholog |
| chr17: 14248391-14248721 | 0 | Mouse ExE ortholog |
| chr17: 32484007-32484280 | 15 | Mouse ExE ortholog |
| chr17: 35291899-35300875 | 5 | Mouse ExE ortholog |
| chr17: 37764092-37764304 | 3 | Mouse ExE ortholog |
| chr17: 40937258-40937480 | 9 | Mouse ExE ortholog |
| chr17: 43472527-43474343 | 0 | Mouse ExE ortholog |
| chr17: 45949676-45949885 | 2 | Mouse ExE ortholog |
| chr17: 46607804-46608390 | 5 | Mouse ExE ortholog |
| chr17: 46620367-46621373 | 1 | Mouse ExE ortholog |
| chr17: 46631800-46632212 | 6 | Mouse ExE ortholog |
| chr17: 46669434-46669811 | 8 | Mouse ExE ortholog |
| chr17: 46691520-46692097 | 7 | Mouse ExE ortholog |
| chr17: 48194634-48195085 | 0 | Mouse ExE ortholog |
| chr17: 50235175-50236466 | 12 | Mouse ExE ortholog |
| chr17: 59485573-59485780 | 11 | Mouse ExE ortholog |
| chr17: 59528979-59530266 | 14 | Mouse ExE ortholog |
| chr17: 70116274-70119998 | 1 | Mouse ExE ortholog |
| chr17: 70120139-70120442 | 7 | Mouse ExE ortholog |
| chr17: 72855621-72858012 | 0 | Mouse ExE ortholog |
| chr17: 72915568-72916510 | 4 | Mouse ExE ortholog |
| chr17: 74017769-74018658 | 5 | Mouse ExE ortholog |
| chr17: 77805866-77809046 | 0 | Mouse ExE ortholog |
| chr17: 79314962-79320653 | 0 | Mouse ExE ortholog |
| chr17: 79859808-79860963 | 1 | Mouse ExE ortholog |
| chr18: 19744936-19752363 | 1 | Mouse ExE ortholog |
| chr18: 30349690-30352302 | 2 | Mouse ExE ortholog |
| chr18: 35144907-35147628 | 6 | Mouse ExE ortholog |
| chr18: 55103154-55108853 | 6 | Mouse ExE ortholog |
| chr18: 55922987-55924068 | 1 | Mouse ExE ortholog |
| chr18: 59000683-59001692 | 12 | Mouse ExE ortholog |
| chr18: 74153239-74155073 | 0 | Mouse ExE ortholog |
| chr18: 74961556-74963822 | 13 | Mouse ExE ortholog |
| chr19: 407011-409511 | 0 | Mouse ExE ortholog |
| chr19: 1063544-1064265 | 4 | Mouse ExE ortholog |
| chr19: 1108394-1109610 | 2 | Mouse ExE ortholog |
| chr19: 1748167-1750243 | 1 | Mouse ExE ortholog |
| chr19: 2424005-2427983 | 0 | Mouse ExE ortholog |
| chr19: 7933263-7934898 | 0 | Mouse ExE ortholog |
| chr19: 11594372-11594987 | 1 | Mouse ExE ortholog |
| chr19: 13135317-13136169 | 8 | Mouse ExE ortholog |
| chr19: 13198699-13198999 | 2 | Mouse ExE ortholog |
| chr19: 13213450-13213821 | 2 | Mouse ExE ortholog |
| chr19: 18979351-18981200 | 13 | Mouse ExE ortholog |
| chr19: 19368708-19369681 | 0 | Mouse ExE ortholog |
| chr19: 30715549-30715753 | 0 | Mouse ExE ortholog |
| chr19: 35633409-35633697 | 1 | Mouse ExE ortholog |
| chr19: 36336275-36337138 | 2 | Mouse ExE ortholog |
| chr19: 36500169-36500530 | 1 | Mouse ExE ortholog |
| chr19: 38876070-38876332 | 0 | Mouse ExE ortholog |
| chr19: 42891311-42891646 | 0 | Mouse ExE ortholog |
| chr19: 45898879-45900315 | 4 | Mouse ExE ortholog |
| chr19: 48965002-48965792 | 2 | Mouse ExE ortholog |
| chr19: 50881418-50881664 | 3 | Mouse ExE ortholog |
| chr19: 50931270-50931638 | 3 | Mouse ExE ortholog |
| chr19: 51169659-51172023 | 9 | Mouse ExE ortholog |
| chr19: 55815940-55816277 | 0 | Mouse ExE ortholog |
| chr19: 56598038-56600296 | 2 | Mouse ExE ortholog |
| chr2: 3750828-3751927 | 8 | Mouse ExE ortholog |
| chr2: 30453566-30455655 | 1 | Mouse ExE ortholog |
| chr2: 38301276-38304518 | 0 | Mouse ExE ortholog |
| chr2: 45155195-45157049 | 12 | Mouse ExE ortholog |
| chr2: 45395869-45398186 | 5 | Mouse ExE ortholog |
| chr2: 50574045-50574817 | 10 | Mouse ExE ortholog |
| chr2: 66808568-66809404 | 13 | Mouse ExE ortholog |
| chr2: 71787430-71787897 | 3 | Mouse ExE ortholog |
| chr2: 73143055-73148260 | 1 | Mouse ExE ortholog |
| chr2: 80529677-80530846 | 11 | Mouse ExE ortholog |
| chr2: 102803672-102804556 | 2 | Mouse ExE ortholog |
| chr2: 105459127-105461770 | 12 | Mouse ExE ortholog |
| chr2: 105468851-105473488 | 4 | Mouse ExE ortholog |
| chr2: 108602824-108603467 | 11 | Mouse ExE ortholog |
| chr2: 119599458-119600966 | 12 | Mouse ExE ortholog |
| chr2: 137522460-137523696 | 8 | Mouse ExE ortholog |
| chr2: 142887724-142888553 | 7 | Mouse ExE ortholog |
| chr2: 144694666-144695180 | 3 | Mouse ExE ortholog |
| chr2: 157185557-157186355 | 7 | Mouse ExE ortholog |
| chr2: 162273294-162273725 | 8 | Mouse ExE ortholog |
| chr2: 176949511-176949795 | 9 | Mouse ExE ortholog |
| chr2: 176964062-176965509 | 12 | Mouse ExE ortholog |
| chr2: 176969217-176969895 | 6 | Mouse ExE ortholog |
| chr2: 176977284-176977540 | 13 | Mouse ExE ortholog |
| chr2: 176982107-176982402 | 12 | Mouse ExE ortholog |
| chr2: 177036254-177037213 | 14 | Mouse ExE ortholog |
| chr2: 177042751-177043444 | 2 | Mouse ExE ortholog |
| chr2: 182321761-182323029 | 10 | Mouse ExE ortholog |
| chr2: 182521221-182521927 | 2 | Mouse ExE ortholog |
| chr2: 219736132-219736592 | 8 | Mouse ExE ortholog |
| chr2: 219848919-219850541 | 6 | Mouse ExE ortholog |
| chr2: 219857682-219858917 | 1 | Mouse ExE ortholog |
| chr2: 220299483-220300243 | 10 | Mouse ExE ortholog |
| chr2: 220412341-220412678 | 0 | Mouse ExE ortholog |
| chr2: 223183013-223185468 | 0 | Mouse ExE ortholog |
| chr2: 237071794-237078762 | 7 | Mouse ExE ortholog |
| chr2: 241758141-241760783 | 3 | Mouse ExE ortholog |
| chr20: 3145121-3145746 | 5 | Mouse ExE ortholog |
| chr20: 21485932-21496714 | 5 | Mouse ExE ortholog |
| chr20: 21686199-21687689 | 12 | Mouse ExE ortholog |
| chr20: 22557517-22559240 | 8 | Mouse ExE ortholog |
| chr20: 33296514-33298242 | 0 | Mouse ExE ortholog |
| chr20: 37352130-37357372 | 13 | Mouse ExE ortholog |
| chr20: 39994545-39995810 | 7 | Mouse ExE ortholog |
| chr20: 44657463-44659243 | 12 | Mouse ExE ortholog |
| chr20: 44685771-44687610 | 9 | Mouse ExE ortholog |
| chr20: 51589707-51590020 | 4 | Mouse ExE ortholog |
| chr20: 52789252-52790986 | 3 | Mouse ExE ortholog |
| chr20: 57415135-57417153 | 5 | Mouse ExE ortholog |
| chr21: 31311386-31312106 | 11 | Mouse ExE ortholog |
| chr21: 32624144-32624382 | 0 | Mouse ExE ortholog |
| chr21: 38065179-38066185 | 10 | Mouse ExE ortholog |
| chr22: 19967279-19967808 | 0 | Mouse ExE ortholog |
| chr22: 29709281-29712013 | 0 | Mouse ExE ortholog |
| chr22: 31198491-31199033 | 1 | Mouse ExE ortholog |
| chr22: 31500396-31501239 | 12 | Mouse ExE ortholog |
| chr22: 37212769-37213467 | 0 | Mouse ExE ortholog |
| chr22: 37911979-37912258 | 1 | Mouse ExE ortholog |
| chr22: 38476836-38478839 | 6 | Mouse ExE ortholog |
| chr22: 42305617-42307254 | 0 | Mouse ExE ortholog |
| chr22: 42322043-42322909 | 3 | Mouse ExE ortholog |
| chr22: 44726724-44727590 | 1 | Mouse ExE ortholog |
| chr22: 46318693-46319087 | 1 | Mouse ExE ortholog |
| chr22: 46440393-46441019 | 1 | Mouse ExE ortholog |
| chr3: 3840513-3842772 | 8 | Mouse ExE ortholog |
| chr3: 6902823-6903516 | 13 | Mouse ExE ortholog |
| chr3: 13114627-13115245 | 11 | Mouse ExE ortholog |
| chr3: 19189688-19190100 | 6 | Mouse ExE ortholog |
| chr3: 49947621-49948430 | 2 | Mouse ExE ortholog |
| chr3: 55508336-55508708 | 4 | Mouse ExE ortholog |
| chr3: 62354291-62355012 | 13 | Mouse ExE ortholog |
| chr3: 62357639-62359774 | 11 | Mouse ExE ortholog |
| chr3: 71834068-71834653 | 4 | Mouse ExE ortholog |
| chr3: 87841796-87842563 | 6 | Mouse ExE ortholog |
| chr3: 137482964-137484454 | 7 | Mouse ExE ortholog |
| chr3: 137489594-137491004 | 10 | Mouse ExE ortholog |
| chr3: 147108511-147111703 | 13 | Mouse ExE ortholog |
| chr3: 147113608-147114479 | 15 | Mouse ExE ortholog |
| chr3: 147130342-147130577 | 15 | Mouse ExE ortholog |
| chr3: 147131066-147131333 | 14 | Mouse ExE ortholog |
| chr3: 154146347-154146965 | 13 | Mouse ExE ortholog |
| chr3: 157821232-157821604 | 15 | Mouse ExE ortholog |
| chr3: 170303044-170303249 | 12 | Mouse ExE ortholog |
| chr3: 172165372-172166738 | 14 | Mouse ExE ortholog |
| chr4: 4868440-4869173 | 9 | Mouse ExE ortholog |
| chr4: 25090106-25090510 | 12 | Mouse ExE ortholog |
| chr4: 41749184-41749811 | 13 | Mouse ExE ortholog |
| chr4: 47034427-47034940 | 7 | Mouse ExE ortholog |
| chr4: 54966163-54968063 | 3 | Mouse ExE ortholog |
| chr4: 81119095-81119391 | 11 | Mouse ExE ortholog |
| chr4: 90228714-90229010 | 1 | Mouse ExE ortholog |
| chr4: 94755786-94756310 | 13 | Mouse ExE ortholog |
| chr4: 100870377-100871994 | 0 | Mouse ExE ortholog |
| chr4: 107956555-107957453 | 11 | Mouse ExE ortholog |
| chr4: 109093038-109094546 | 3 | Mouse ExE ortholog |
| chr4: 114900355-114900810 | 2 | Mouse ExE ortholog |
| chr4: 122301567-122302290 | 10 | Mouse ExE ortholog |
| chr4: 128544031-128544903 | 10 | Mouse ExE ortholog |
| chr4: 144620822-144622218 | 9 | Mouse ExE ortholog |
| chr4: 147559205-147561901 | 14 | Mouse ExE ortholog |
| chr4: 156680095-156681386 | 6 | Mouse ExE ortholog |
| chr4: 164264821-164265772 | 10 | Mouse ExE ortholog |
| chr4: 172733734-172735118 | 11 | Mouse ExE ortholog |
| chr4: 174430386-174430861 | 14 | Mouse ExE ortholog |
| chr4: 185939222-185942747 | 4 | Mouse ExE ortholog |
| chr5: 1879689-1879928 | 10 | Mouse ExE ortholog |
| chr5: 1881924-1887743 | 8 | Mouse ExE ortholog |
| chr5: 2748368-2757024 | 7 | Mouse ExE ortholog |
| chr5: 37834671-37835128 | 12 | Mouse ExE ortholog |
| chr5: 38257825-38259136 | 4 | Mouse ExE ortholog |
| chr5: 52777788-52777996 | 0 | Mouse ExE ortholog |
| chr5: 54527319-54527760 | 0 | Mouse ExE ortholog |
| chr5: 59189046-59189894 | 7 | Mouse ExE ortholog |
| chr5: 63256548-63257886 | 12 | Mouse ExE ortholog |
| chr5: 71014917-71015715 | 13 | Mouse ExE ortholog |
| chr5: 72529099-72529976 | 7 | Mouse ExE ortholog |
| chr5: 76932317-76933523 | 10 | Mouse ExE ortholog |
| chr5: 76934581-76935296 | 12 | Mouse ExE ortholog |
| chr5: 77805753-77806313 | 2 | Mouse ExE ortholog |
| chr5: 92923487-92924497 | 14 | Mouse ExE ortholog |
| chr5: 92939795-92940216 | 12 | Mouse ExE ortholog |
| chr5: 134363092-134365146 | 12 | Mouse ExE ortholog |
| chr5: 134366913-134367438 | 7 | Mouse ExE ortholog |
| chr5: 134374385-134376751 | 3 | Mouse ExE ortholog |
| chr5: 139138875-139139242 | 4 | Mouse ExE ortholog |
| chr5: 140052059-140053381 | 0 | Mouse ExE ortholog |
| chr5: 140305712-140307193 | 10 | Mouse ExE ortholog |
| chr5: 140798757-140799359 | 15 | Mouse ExE ortholog |
| chr5: 140810494-140812617 | 15 | Mouse ExE ortholog |
| chr5: 145718289-145720095 | 11 | Mouse ExE ortholog |
| chr5: 145725286-145725852 | 13 | Mouse ExE ortholog |
| chr5: 158523906-158524598 | 7 | Mouse ExE ortholog |
| chr5: 172665306-172666072 | 4 | Mouse ExE ortholog |
| chr5: 179228283-179229003 | 9 | Mouse ExE ortholog |
| chr6: 391188-393790 | 13 | Mouse ExE ortholog |
| chr6: 1381743-1385211 | 6 | Mouse ExE ortholog |
| chr6: 5997027-5997414 | 2 | Mouse ExE ortholog |
| chr6: 6007387-6007797 | 4 | Mouse ExE ortholog |
| chr6: 7229877-7230865 | 0 | Mouse ExE ortholog |
| chr6: 10390038-10390565 | 8 | Mouse ExE ortholog |
| chr6: 29894140-29895117 | 13 | Mouse ExE ortholog |
| chr6: 33393592-33393908 | 0 | Mouse ExE ortholog |
| chr6: 33655966-33656238 | 1 | Mouse ExE ortholog |
| chr6: 41908745-41909711 | 0 | Mouse ExE ortholog |
| chr6: 42072032-42072701 | 10 | Mouse ExE ortholog |
| chr6: 46655262-46656738 | 0 | Mouse ExE ortholog |
| chr6: 50682334-50683214 | 14 | Mouse ExE ortholog |
| chr6: 50791110-50791573 | 12 | Mouse ExE ortholog |
| chr6: 55039170-55039392 | 12 | Mouse ExE ortholog |
| chr6: 99275763-99276038 | 7 | Mouse ExE ortholog |
| chr6: 101846766-101847135 | 11 | Mouse ExE ortholog |
| chr6: 108485671-108490539 | 12 | Mouse ExE ortholog |
| chr6: 108491033-108491410 | 12 | Mouse ExE ortholog |
| chr6: 108497595-108497996 | 9 | Mouse ExE ortholog |
| chr6: 117198089-117198705 | 3 | Mouse ExE ortholog |
| chr6: 117591533-117592279 | 9 | Mouse ExE ortholog |
| chr6: 134210639-134211218 | 10 | Mouse ExE ortholog |
| chr6: 134638797-134639021 | 5 | Mouse ExE ortholog |
| chr6: 137242315-137245442 | 0 | Mouse ExE ortholog |
| chr6: 137814355-137815202 | 13 | Mouse ExE ortholog |
| chr6: 138745348-138745593 | 0 | Mouse ExE ortholog |
| chr7: 1362811-1363643 | 0 | Mouse ExE ortholog |
| chr7: 6590563-6590957 | 2 | Mouse ExE ortholog |
| chr7: 6661875-6662695 | 0 | Mouse ExE ortholog |
| chr7: 19145872-19146256 | 12 | Mouse ExE ortholog |
| chr7: 20370003-20371504 | 0 | Mouse ExE ortholog |
| chr7: 20830567-20830817 | 6 | Mouse ExE ortholog |
| chr7: 26415746-26416891 | 2 | Mouse ExE ortholog |
| chr7: 27146069-27146600 | 13 | Mouse ExE ortholog |
| chr7: 27182613-27185562 | 8 | Mouse ExE ortholog |
| chr7: 27227520-27229043 | 3 | Mouse ExE ortholog |
| chr7: 27278945-27279469 | 13 | Mouse ExE ortholog |
| chr7: 27282086-27283136 | 7 | Mouse ExE ortholog |
| chr7: 30721372-30722445 | 12 | Mouse ExE ortholog |
| chr7: 37955622-37956555 | 2 | Mouse ExE ortholog |
| chr7: 49813008-49815752 | 11 | Mouse ExE ortholog |
| chr7: 56355508-56355798 | 13 | Mouse ExE ortholog |
| chr7: 87563342-87564571 | 0 | Mouse ExE ortholog |
| chr7: 90893567-90896683 | 0 | Mouse ExE ortholog |
| chr7: 95225503-95226194 | 2 | Mouse ExE ortholog |
| chr7: 96650221-96651551 | 12 | Mouse ExE ortholog |
| chr7: 96651963-96652246 | 10 | Mouse ExE ortholog |
| chr7: 97841636-97842005 | 0 | Mouse ExE ortholog |
| chr7: 113724924-113727795 | 1 | Mouse ExE ortholog |
| chr7: 130790358-130792773 | 0 | Mouse ExE ortholog |
| chr7: 136553854-136556194 | 12 | Mouse ExE ortholog |
| chr7: 155595692-155599414 | 6 | Mouse ExE ortholog |
| chr7: 155604725-155605095 | 3 | Mouse ExE ortholog |
| chr7: 156795355-156799394 | 11 | Mouse ExE ortholog |
| chr8: 21905461-21905757 | 6 | Mouse ExE ortholog |
| chr8: 25900562-25905842 | 3 | Mouse ExE ortholog |
| chr8: 55366180-55367628 | 11 | Mouse ExE ortholog |
| chr8: 65710990-65711722 | 6 | Mouse ExE ortholog |
| chr8: 70981873-70984888 | 14 | Mouse ExE ortholog |
| chr8: 105478672-105479340 | 13 | Mouse ExE ortholog |
| chr8: 120428398-120429178 | 1 | Mouse ExE ortholog |
| chr8: 143545445-143546178 | 5 | Mouse ExE ortholog |
| chr8: 144808221-144810978 | 1 | Mouse ExE ortholog |
| chr8: 144990270-145002135 | 0 | Mouse ExE ortholog |
| chr9: 17906419-17907488 | 11 | Mouse ExE ortholog |
| chr9: 21970913-21971190 | 1 | Mouse ExE ortholog |
| chr9: 22005887-22006229 | 0 | Mouse ExE ortholog |
| chr9: 86152353-86153777 | 0 | Mouse ExE ortholog |
| chr9: 95477296-95477708 | 5 | Mouse ExE ortholog |
| chr9: 96713326-96718186 | 9 | Mouse ExE ortholog |
| chr9: 97401286-97402067 | 0 | Mouse ExE ortholog |
| chr9: 102590742-102591303 | 12 | Mouse ExE ortholog |
| chr9: 112081402-112082905 | 1 | Mouse ExE ortholog |
| chr9: 120175253-120177496 | 8 | Mouse ExE ortholog |
| chr9: 122131086-122132214 | 11 | Mouse ExE ortholog |
| chr9: 124413512-124414193 | 0 | Mouse ExE ortholog |
| chr9: 124987743-124991086 | 4 | Mouse ExE ortholog |
| chr9: 126773246-126780953 | 8 | Mouse ExE ortholog |
| chr9: 129372737-129378106 | 3 | Mouse ExE ortholog |
| chr9: 129386112-129389231 | 7 | Mouse ExE ortholog |
| chr9: 131154346-131155923 | 2 | Mouse ExE ortholog |
| chr9: 132459587-132460017 | 4 | Mouse ExE ortholog |
| chr9: 133534534-133542394 | 9 | Mouse ExE ortholog |
| chr9: 135039673-135039978 | 3 | Mouse ExE ortholog |
| chr9: 135455164-135458586 | 4 | Mouse ExE ortholog |
| chr9: 135461934-135462909 | 13 | Mouse ExE ortholog |
| chr9: 135464586-135466240 | 6 | Mouse ExE ortholog |
| chr9: 139096665-139096993 | 6 | Mouse ExE ortholog |
| chr9: 139396205-139397040 | 0 | Mouse ExE ortholog |
| chrX: 67352650-67352923 | 0 | Mouse ExE ortholog |
| chrX: 99891299-99891794 | 0 | Mouse ExE ortholog |
| chrX: 152612775-152613464 | 0 | Mouse ExE ortholog |
| chr1: 1474962-1475220 | 14 | Human Placenta |
| chr1: 2979275-2980758 | 8 | Human Placenta |
| chr1: 10764449-10764925 | 9 | Human Placenta |
| chr1: 12123488-12124148 | 8 | Human Placenta |
| chr1: 16860873-16862296 | 14 | Human Placenta |
| chr1: 18964180-18964401 | 9 | Human Placenta |
| chr1: 24229115-24229537 | 14 | Human Placenta |
| chr1: 32052471-32052771 | 9 | Human Placenta |
| chr1: 34642382-34643024 | 14 | Human Placenta |
| chr1: 36549554-36549965 | 11 | Human Placenta |
| chr1: 38219702-38220012 | 9 | Human Placenta |
| chr1: 38461584-38461988 | 8 | Human Placenta |
| chr1: 38941919-38942404 | 11 | Human Placenta |
| chr1: 39044059-39044561 | 8 | Human Placenta |
| chr1: 40769186-40769871 | 9 | Human Placenta |
| chr1: 41284847-41285149 | 9 | Human Placenta |
| chr1: 44031286-44031853 | 14 | Human Placenta |
| chr1: 47009575-47010132 | 13 | Human Placenta |
| chr1: 50880916-50881516 | 14 | Human Placenta |
| chr1: 50881884-50882103 | 12 | Human Placenta |
| chr1: 50892437-50893243 | 10 | Human Placenta |
| chr1: 53527572-53528974 | 9 | Human Placenta |
| chr1: 63795363-63796140 | 13 | Human Placenta |
| chr1: 65991001-65991811 | 11 | Human Placenta |
| chr1: 67218079-67218293 | 14 | Human Placenta |
| chr1: 67773329-67773767 | 9 | Human Placenta |
| chr1: 86621278-86622871 | 8 | Human Placenta |
| chr1: 91183240-91184540 | 15 | Human Placenta |
| chr1: 91185156-91185577 | 15 | Human Placenta |
| chr1: 91190489-91192804 | 11 | Human Placenta |
| chr1: 91300979-91301891 | 15 | Human Placenta |
| chr1: 110610265-110613303 | 11 | Human Placenta |
| chr1: 113265573-113265787 | 8 | Human Placenta |
| chr1: 113286332-113287172 | 9 | Human Placenta |
| chr1: 114695136-114696672 | 10 | Human Placenta |
| chr1: 119526782-119527192 | 15 | Human Placenta |
| chr1: 119529819-119530712 | 13 | Human Placenta |
| chr1: 119543056-119543454 | 14 | Human Placenta |
| chr1: 119549144-119551320 | 13 | Human Placenta |
| chr1: 145075483-145075845 | 13 | Human Placenta |
| chr1: 146552328-146552577 | 13 | Human Placenta |
| chr1: 147782066-147782473 | 13 | Human Placenta |
| chr1: 149332993-149333389 | 14 | Human Placenta |
| chr1: 155147185-155147444 | 9 | Human Placenta |
| chr1: 155264318-155265536 | 14 | Human Placenta |
| chr1: 155290606-155291001 | 10 | Human Placenta |
| chr1: 156863415-156863711 | 14 | Human Placenta |
| chr1: 164545540-164545917 | 12 | Human Placenta |
| chr1: 165324191-165326328 | 11 | Human Placenta |
| chr1: 170630456-170630851 | 13 | Human Placenta |
| chr1: 173638662-173639045 | 12 | Human Placenta |
| chr1: 175568376-175568808 | 12 | Human Placenta |
| chr1: 179544720-179545307 | 8 | Human Placenta |
| chr1: 181287300-181287873 | 8 | Human Placenta |
| chr1: 181452706-181453073 | 9 | Human Placenta |
| chr1: 200009807-200010036 | 8 | Human Placenta |
| chr1: 202162958-202163390 | 12 | Human Placenta |
| chr1: 203044722-203045390 | 12 | Human Placenta |
| chr1: 208132327-208133117 | 11 | Human Placenta |
| chr1: 214153214-214153668 | 13 | Human Placenta |
| chr1: 217310749-217311178 | 14 | Human Placenta |
| chr1: 221050448-221050864 | 13 | Human Placenta |
| chr1: 221060850-221061071 | 10 | Human Placenta |
| chr1: 225865068-225865328 | 12 | Human Placenta |
| chr1: 226127112-226127695 | 12 | Human Placenta |
| chr1: 228785986-228786204 | 15 | Human Placenta |
| chr1: 231296559-231297345 | 12 | Human Placenta |
| chr1: 243646394-243646888 | 9 | Human Placenta |
| chr10: 1778784-1780018 | 8 | Human Placenta |
| chr10: 8076002-8077261 | 8 | Human Placenta |
| chr10: 8077829-8078378 | 12 | Human Placenta |
| chr10: 15761423-15762101 | 13 | Human Placenta |
| chr10: 16561604-16563822 | 10 | Human Placenta |
| chr10: 22623350-22625875 | 9 | Human Placenta |
| chr10: 22634000-22634862 | 15 | Human Placenta |
| chr10: 22764708-22767050 | 12 | Human Placenta |
| chr10: 23461300-23461610 | 14 | Human Placenta |
| chr10: 23462224-23463889 | 12 | Human Placenta |
| chr10: 23480697-23482455 | 12 | Human Placenta |
| chr10: 23983366-23984978 | 10 | Human Placenta |
| chr10: 26504383-26507434 | 11 | Human Placenta |
| chr10: 27547668-27548402 | 9 | Human Placenta |
| chr10: 43428167-43429460 | 8 | Human Placenta |
| chr10: 48438411-48439320 | 11 | Human Placenta |
| chr10: 63212495-63213009 | 9 | Human Placenta |
| chr10: 71331449-71331691 | 13 | Human Placenta |
| chr10: 75407413-75407706 | 11 | Human Placenta |
| chr10: 76573195-76573507 | 15 | Human Placenta |
| chr10: 94180315-94180754 | 13 | Human Placenta |
| chr10: 94455524-94455896 | 8 | Human Placenta |
| chr10: 94828102-94829040 | 13 | Human Placenta |
| chr10: 99789614-99791320 | 8 | Human Placenta |
| chr10: 100992156-100992687 | 12 | Human Placenta |
| chr10: 101282725-101282934 | 8 | Human Placenta |
| chr10: 101290025-101290338 | 14 | Human Placenta |
| chr10: 102279162-102279730 | 12 | Human Placenta |
| chr10: 102475276-102475579 | 8 | Human Placenta |
| chr10: 102891010-102891794 | 12 | Human Placenta |
| chr10: 102905714-102906693 | 11 | Human Placenta |
| chr10: 102996034-102996646 | 12 | Human Placenta |
| chr10: 103043990-103044480 | 12 | Human Placenta |
| chr10: 108923780-108924805 | 12 | Human Placenta |
| chr10: 109674196-109674964 | 13 | Human Placenta |
| chr10: 110671724-110672326 | 12 | Human Placenta |
| chr10: 111216604-111217083 | 12 | Human Placenta |
| chr10: 118030732-118034230 | 9 | Human Placenta |
| chr10: 118892161-118892639 | 12 | Human Placenta |
| chr10: 118893527-118894432 | 12 | Human Placenta |
| chr10: 119494493-119494991 | 10 | Human Placenta |
| chr10: 120353692-120355821 | 12 | Human Placenta |
| chr10: 121577529-121578385 | 15 | Human Placenta |
| chr10: 123922850-123923542 | 13 | Human Placenta |
| chr10: 124901907-124902617 | 12 | Human Placenta |
| chr10: 125425495-125426642 | 14 | Human Placenta |
| chr10: 125650820-125651373 | 11 | Human Placenta |
| chr10: 125732220-125732843 | 14 | Human Placenta |
| chr10: 130338695-130338994 | 12 | Human Placenta |
| chr10: 130508443-130508658 | 10 | Human Placenta |
| chr10: 134597357-134602649 | 11 | Human Placenta |
| chr11: 626728-628037 | 9 | Human Placenta |
| chr11: 636435-636668 | 9 | Human Placenta |
| chr11: 636906-640628 | 8 | Human Placenta |
| chr11: 2890388-2891337 | 8 | Human Placenta |
| chr11: 14995128-14995908 | 10 | Human Placenta |
| chr11: 20618197-20619920 | 14 | Human Placenta |
| chr11: 27743472-27744564 | 12 | Human Placenta |
| chr11: 31827696-31827921 | 14 | Human Placenta |
| chr11: 31841315-31842003 | 14 | Human Placenta |
| chr11: 31847132-31847958 | 13 | Human Placenta |
| chr11: 43568921-43569854 | 10 | Human Placenta |
| chr11: 44325657-44326517 | 9 | Human Placenta |
| chr11: 60718428-60718888 | 9 | Human Placenta |
| chr11: 64478843-64479598 | 14 | Human Placenta |
| chr11: 64815040-64815722 | 13 | Human Placenta |
| chr11: 65409636-65410127 | 13 | Human Placenta |
| chr11: 65816404-65816665 | 14 | Human Placenta |
| chr11: 68622108-68622339 | 9 | Human Placenta |
| chr11: 70508328-70508617 | 9 | Human Placenta |
| chr11: 71952112-71952528 | 11 | Human Placenta |
| chr11: 88241710-88242562 | 10 | Human Placenta |
| chr11: 89224416-89224718 | 11 | Human Placenta |
| chr11: 105481126-105481422 | 12 | Human Placenta |
| chr11: 115630398-115631117 | 8 | Human Placenta |
| chr11: 119293320-119293943 | 13 | Human Placenta |
| chr11: 123066517-123066986 | 13 | Human Placenta |
| chr11: 128419198-128419513 | 10 | Human Placenta |
| chr11: 128694084-128694688 | 8 | Human Placenta |
| chr11: 131780328-131781532 | 12 | Human Placenta |
| chr11: 132813562-132814395 | 9 | Human Placenta |
| chr11: 132934059-132934291 | 11 | Human Placenta |
| chr11: 132952538-132953307 | 8 | Human Placenta |
| chr11: 133994709-133995090 | 9 | Human Placenta |
| chr12: 186863-187610 | 9 | Human Placenta |
| chr12: 3308812-3310270 | 8 | Human Placenta |
| chr12: 5153012-5154346 | 9 | Human Placenta |
| chr12: 14134626-14135242 | 8 | Human Placenta |
| chr12: 41086522-41087102 | 9 | Human Placenta |
| chr12: 48399168-48399372 | 8 | Human Placenta |
| chr12: 52115410-52115679 | 11 | Human Placenta |
| chr12: 52408381-52408675 | 13 | Human Placenta |
| chr12: 52652018-52652743 | 13 | Human Placenta |
| chr12: 53107912-53108471 | 10 | Human Placenta |
| chr12: 53359192-53359507 | 12 | Human Placenta |
| chr12: 54071053-54071265 | 10 | Human Placenta |
| chr12: 54321301-54321721 | 12 | Human Placenta |
| chr12: 54354529-54355491 | 12 | Human Placenta |
| chr12: 54359658-54359906 | 12 | Human Placenta |
| chr12: 54424610-54425173 | 8 | Human Placenta |
| chr12: 65218245-65219143 | 8 | Human Placenta |
| chr12: 65514878-65515863 | 9 | Human Placenta |
| chr12: 72665683-72667551 | 10 | Human Placenta |
| chr12: 81102034-81102716 | 14 | Human Placenta |
| chr12: 81471569-81472119 | 10 | Human Placenta |
| chr12: 103696090-103696418 | 13 | Human Placenta |
| chr12: 104697348-104697984 | 11 | Human Placenta |
| chr12: 106974412-106974951 | 11 | Human Placenta |
| chr12: 113013099-113013529 | 14 | Human Placenta |
| chr12: 113515164-113515970 | 9 | Human Placenta |
| chr12: 113916433-113916717 | 14 | Human Placenta |
| chr12: 114833911-114834210 | 9 | Human Placenta |
| chr12: 114838312-114838889 | 8 | Human Placenta |
| chr12: 114843022-114843610 | 10 | Human Placenta |
| chr12: 114845861-114847650 | 14 | Human Placenta |
| chr12: 114851957-114852360 | 9 | Human Placenta |
| chr12: 114881649-114881937 | 10 | Human Placenta |
| chr12: 114885105-114885418 | 13 | Human Placenta |
| chr12: 119212110-119212393 | 10 | Human Placenta |
| chr12: 123754049-123754373 | 14 | Human Placenta |
| chr12: 127210778-127211651 | 14 | Human Placenta |
| chr12: 127940451-127940907 | 14 | Human Placenta |
| chr12: 129337870-129338653 | 11 | Human Placenta |
| chr12: 131199824-131200157 | 8 | Human Placenta |
| chr12: 132905449-132906206 | 9 | Human Placenta |
| chr13: 20875518-20876214 | 9 | Human Placenta |
| chr13: 28366549-28368505 | 9 | Human Placenta |
| chr13: 28549839-28550246 | 13 | Human Placenta |
| chr13: 36044844-36045481 | 13 | Human Placenta |
| chr13: 51417371-51418149 | 14 | Human Placenta |
| chr13: 53419897-53422872 | 13 | Human Placenta |
| chr13: 58203586-58204322 | 11 | Human Placenta |
| chr13: 58206526-58208930 | 14 | Human Placenta |
| chr13: 79181944-79182222 | 14 | Human Placenta |
| chr13: 93879245-93880877 | 8 | Human Placenta |
| chr13: 100547633-100548911 | 8 | Human Placenta |
| chr13: 100641334-100642188 | 12 | Human Placenta |
| chr13: 102568425-102569495 | 10 | Human Placenta |
| chr13: 112707804-112708696 | 13 | Human Placenta |
| chr13: 112709884-112712665 | 14 | Human Placenta |
| chr13: 112715359-112716234 | 10 | Human Placenta |
| chr13: 112717125-112717421 | 14 | Human Placenta |
| chr13: 112720564-112723582 | 14 | Human Placenta |
| chr13: 112726281-112728419 | 11 | Human Placenta |
| chr13: 112758598-112760491 | 14 | Human Placenta |
| chr13: 112760865-112761113 | 8 | Human Placenta |
| chr14: 24044886-24046760 | 8 | Human Placenta |
| chr14: 24641053-24642220 | 13 | Human Placenta |
| chr14: 24803678-24804353 | 12 | Human Placenta |
| chr14: 29236835-29237832 | 15 | Human Placenta |
| chr14: 29254365-29255069 | 14 | Human Placenta |
| chr14: 33402094-33404079 | 8 | Human Placenta |
| chr14: 36973169-36973740 | 13 | Human Placenta |
| chr14: 36983440-36983738 | 11 | Human Placenta |
| chr14: 36990873-36991209 | 12 | Human Placenta |
| chr14: 36993488-36994488 | 9 | Human Placenta |
| chr14: 37053134-37053690 | 14 | Human Placenta |
| chr14: 37126786-37128274 | 8 | Human Placenta |
| chr14: 37135513-37136348 | 10 | Human Placenta |
| chr14: 38724254-38725537 | 15 | Human Placenta |
| chr14: 48143433-48145589 | 13 | Human Placenta |
| chr14: 51338712-51339146 | 11 | Human Placenta |
| chr14: 52734207-52735486 | 12 | Human Placenta |
| chr14: 57260878-57262123 | 12 | Human Placenta |
| chr14: 57264638-57265561 | 12 | Human Placenta |
| chr14: 57278709-57279116 | 14 | Human Placenta |
| chr14: 58331676-58333121 | 11 | Human Placenta |
| chr14: 60973772-60974123 | 13 | Human Placenta |
| chr14: 60975732-60978180 | 12 | Human Placenta |
| chr14: 61103978-61104663 | 12 | Human Placenta |
| chr14: 62279476-62280019 | 9 | Human Placenta |
| chr14: 77736733-77737772 | 11 | Human Placenta |
| chr14: 85997468-85998637 | 10 | Human Placenta |
| chr14: 85999532-86000478 | 8 | Human Placenta |
| chr14: 92789494-92790712 | 9 | Human Placenta |
| chr14: 95239375-95239679 | 14 | Human Placenta |
| chr14: 95826675-95826941 | 9 | Human Placenta |
| chr14: 101192851-101193499 | 11 | Human Placenta |
| chr14: 101923575-101925995 | 11 | Human Placenta |
| chr14: 103655241-103655928 | 11 | Human Placenta |
| chr15: 23157794-23158624 | 12 | Human Placenta |
| chr15: 27112030-27113479 | 12 | Human Placenta |
| chr15: 27215951-27216856 | 12 | Human Placenta |
| chr15: 33602816-33604003 | 10 | Human Placenta |
| chr15: 35046443-35047480 | 12 | Human Placenta |
| chr15: 37390175-37390380 | 13 | Human Placenta |
| chr15: 53076187-53077926 | 11 | Human Placenta |
| chr15: 53079220-53079579 | 13 | Human Placenta |
| chr15: 53080458-53083699 | 12 | Human Placenta |
| chr15: 53087211-53087488 | 9 | Human Placenta |
| chr15: 53097561-53098476 | 13 | Human Placenta |
| chr15: 59157045-59157594 | 11 | Human Placenta |
| chr15: 76630029-76630970 | 11 | Human Placenta |
| chr15: 79574830-79575211 | 8 | Human Placenta |
| chr15: 89147660-89149198 | 9 | Human Placenta |
| chr15: 89312719-89313183 | 8 | Human Placenta |
| chr15: 89903446-89903720 | 12 | Human Placenta |
| chr15: 89910521-89912177 | 8 | Human Placenta |
| chr15: 89952271-89953061 | 11 | Human Placenta |
| chr15: 96895306-96895729 | 8 | Human Placenta |
| chr15: 96903311-96903711 | 9 | Human Placenta |
| chr15: 96904722-96905050 | 9 | Human Placenta |
| chr15: 96909815-96910030 | 8 | Human Placenta |
| chr15: 96959341-96960531 | 8 | Human Placenta |
| chr15: 100913438-100914022 | 10 | Human Placenta |
| chr16: 3067521-3068358 | 9 | Human Placenta |
| chr16: 3220438-3221356 | 9 | Human Placenta |
| chr16: 6068914-6070401 | 11 | Human Placenta |
| chr16: 10912159-10912719 | 10 | Human Placenta |
| chr16: 20084707-20085305 | 9 | Human Placenta |
| chr16: 23724270-23724775 | 8 | Human Placenta |
| chr16: 24267040-24267527 | 8 | Human Placenta |
| chr16: 31053479-31053800 | 11 | Human Placenta |
| chr16: 49309123-49309353 | 13 | Human Placenta |
| chr16: 49316997-49317263 | 11 | Human Placenta |
| chr16: 51183699-51188763 | 10 | Human Placenta |
| chr16: 54325040-54325703 | 10 | Human Placenta |
| chr16: 55364823-55365483 | 8 | Human Placenta |
| chr16: 66612749-66613412 | 10 | Human Placenta |
| chr16: 67918679-67918909 | 8 | Human Placenta |
| chr16: 71459781-71460338 | 13 | Human Placenta |
| chr16: 82660651-82661813 | 10 | Human Placenta |
| chr16: 84002269-84002860 | 8 | Human Placenta |
| chr17: 934417-935088 | 11 | Human Placenta |
| chr17: 1173535-1174733 | 10 | Human Placenta |
| chr17: 1880789-1881116 | 9 | Human Placenta |
| chr17: 5000369-5001205 | 15 | Human Placenta |
| chr17: 6616422-6617471 | 10 | Human Placenta |
| chr17: 6679205-6679710 | 13 | Human Placenta |
| chr17: 7832532-7833164 | 13 | Human Placenta |
| chr17: 7905927-7907445 | 9 | Human Placenta |
| chr17: 12877270-12877773 | 8 | Human Placenta |
| chr17: 14201726-14202052 | 13 | Human Placenta |
| chr17: 15820620-15821325 | 14 | Human Placenta |
| chr17: 19883325-19883610 | 11 | Human Placenta |
| chr17: 21367114-21367592 | 9 | Human Placenta |
| chr17: 27899511-27900067 | 11 | Human Placenta |
| chr17: 33776553-33776888 | 12 | Human Placenta |
| chr17: 36717727-36718593 | 11 | Human Placenta |
| chr17: 37321482-37322099 | 9 | Human Placenta |
| chr17: 43037166-43037740 | 12 | Human Placenta |
| chr17: 46604362-46604881 | 9 | Human Placenta |
| chr17: 46627787-46628444 | 8 | Human Placenta |
| chr17: 46673532-46674181 | 10 | Human Placenta |
| chr17: 46697413-46697701 | 8 | Human Placenta |
| chr17: 46796234-46797292 | 11 | Human Placenta |
| chr17: 46800533-46800746 | 10 | Human Placenta |
| chr17: 46824785-46825372 | 12 | Human Placenta |
| chr17: 48041282-48043064 | 10 | Human Placenta |
| chr17: 48545570-48546900 | 11 | Human Placenta |
| chr17: 59531723-59535254 | 8 | Human Placenta |
| chr17: 70111979-70112308 | 12 | Human Placenta |
| chr17: 70112824-70114271 | 12 | Human Placenta |
| chr17: 71948478-71949255 | 8 | Human Placenta |
| chr17: 73749618-73750178 | 11 | Human Placenta |
| chr17: 74533281-74534566 | 8 | Human Placenta |
| chr18: 904578-909574 | 13 | Human Placenta |
| chr18: 11148307-11149936 | 13 | Human Placenta |
| chr18: 11750953-11752756 | 9 | Human Placenta |
| chr18: 12254147-12255089 | 8 | Human Placenta |
| chr18: 13641584-13642415 | 8 | Human Placenta |
| chr18: 13868532-13869026 | 12 | Human Placenta |
| chr18: 43608140-43608510 | 10 | Human Placenta |
| chr18: 44336183-44337110 | 11 | Human Placenta |
| chr18: 44337510-44338100 | 10 | Human Placenta |
| chr18: 44772992-44775577 | 15 | Human Placenta |
| chr18: 44777632-44778084 | 14 | Human Placenta |
| chr18: 44789742-44790678 | 14 | Human Placenta |
| chr18: 54788959-54789194 | 12 | Human Placenta |
| chr18: 55019707-55021605 | 15 | Human Placenta |
| chr18: 55094825-55096310 | 14 | Human Placenta |
| chr18: 56887091-56887665 | 9 | Human Placenta |
| chr18: 56939624-56941540 | 10 | Human Placenta |
| chr18: 70533965-70536871 | 11 | Human Placenta |
| chr18: 72916107-72917233 | 10 | Human Placenta |
| chr18: 73167402-73167920 | 12 | Human Placenta |
| chr18: 74799144-74800038 | 10 | Human Placenta |
| chr18: 76732970-76734765 | 11 | Human Placenta |
| chr18: 76737005-76741244 | 13 | Human Placenta |
| chr18: 77547965-77549038 | 14 | Human Placenta |
| chr18: 77557780-77558948 | 10 | Human Placenta |
| chr19: 870774-871318 | 10 | Human Placenta |
| chr19: 3868586-3869217 | 10 | Human Placenta |
| chr19: 5829048-5829474 | 8 | Human Placenta |
| chr19: 8674332-8674764 | 12 | Human Placenta |
| chr19: 10406934-10407342 | 9 | Human Placenta |
| chr19: 10463626-10464378 | 8 | Human Placenta |
| chr19: 12666243-12666682 | 9 | Human Placenta |
| chr19: 12767749-12767980 | 13 | Human Placenta |
| chr19: 12831793-12832225 | 10 | Human Placenta |
| chr19: 12880574-12880888 | 13 | Human Placenta |
| chr19: 13124959-13125259 | 9 | Human Placenta |
| chr19: 13616752-13617267 | 13 | Human Placenta |
| chr19: 14089570-14089796 | 14 | Human Placenta |
| chr19: 19371675-19372393 | 12 | Human Placenta |
| chr19: 21769189-21769786 | 10 | Human Placenta |
| chr19: 33625467-33625805 | 11 | Human Placenta |
| chr19: 36246328-36247982 | 9 | Human Placenta |
| chr19: 36523391-36523887 | 11 | Human Placenta |
| chr19: 38700333-38700577 | 8 | Human Placenta |
| chr19: 39737689-39739288 | 9 | Human Placenta |
| chr19: 39754973-39756540 | 11 | Human Placenta |
| chr19: 40314926-40315144 | 9 | Human Placenta |
| chr19: 44203558-44203987 | 12 | Human Placenta |
| chr19: 44278273-44278777 | 12 | Human Placenta |
| chr19: 45260352-45261809 | 8 | Human Placenta |
| chr19: 46001830-46002686 | 10 | Human Placenta |
| chr19: 46318490-46319266 | 12 | Human Placenta |
| chr19: 46915311-46915802 | 12 | Human Placenta |
| chr19: 47151768-47153125 | 9 | Human Placenta |
| chr19: 49669275-49669552 | 8 | Human Placenta |
| chr19: 51601822-51602260 | 9 | Human Placenta |
| chr19: 51815157-51815458 | 11 | Human Placenta |
| chr19: 54412710-54413087 | 13 | Human Placenta |
| chr19: 54481412-54481955 | 15 | Human Placenta |
| chr19: 54483021-54483572 | 9 | Human Placenta |
| chr19: 55597977-55598887 | 8 | Human Placenta |
| chr19: 56988313-56989741 | 12 | Human Placenta |
| chr19: 58094739-58095764 | 11 | Human Placenta |
| chr19: 58545115-58545897 | 13 | Human Placenta |
| chr19: 58554354-58554587 | 13 | Human Placenta |
| chr2: 467849-468659 | 14 | Human Placenta |
| chr2: 3286324-3286530 | 8 | Human Placenta |
| chr2: 5831187-5831413 | 15 | Human Placenta |
| chr2: 19560963-19561650 | 10 | Human Placenta |
| chr2: 20870006-20871280 | 14 | Human Placenta |
| chr2: 25499763-25500429 | 12 | Human Placenta |
| chr2: 31805293-31806403 | 12 | Human Placenta |
| chr2: 45169505-45171884 | 11 | Human Placenta |
| chr2: 45227644-45228783 | 8 | Human Placenta |
| chr2: 45240372-45241579 | 8 | Human Placenta |
| chr2: 54086776-54087266 | 13 | Human Placenta |
| chr2: 63282514-63283122 | 14 | Human Placenta |
| chr2: 63283936-63284147 | 13 | Human Placenta |
| chr2: 63285949-63287097 | 8 | Human Placenta |
| chr2: 66652691-66654218 | 9 | Human Placenta |
| chr2: 66672431-66673636 | 10 | Human Placenta |
| chr2: 80549578-80549798 | 10 | Human Placenta |
| chr2: 87015974-87018182 | 9 | Human Placenta |
| chr2: 87088816-87089037 | 11 | Human Placenta |
| chr2: 97192977-97193383 | 10 | Human Placenta |
| chr2: 105480197-105480760 | 13 | Human Placenta |
| chr2: 106681982-106682403 | 13 | Human Placenta |
| chr2: 107103833-107104053 | 11 | Human Placenta |
| chr2: 114033359-114033617 | 11 | Human Placenta |
| chr2: 114034594-114036041 | 9 | Human Placenta |
| chr2: 114256775-114258043 | 14 | Human Placenta |
| chr2: 118981769-118982466 | 10 | Human Placenta |
| chr2: 119592602-119593845 | 12 | Human Placenta |
| chr2: 119599059-119599299 | 14 | Human Placenta |
| chr2: 119602616-119604486 | 8 | Human Placenta |
| chr2: 119606569-119606826 | 10 | Human Placenta |
| chr2: 119611296-119611881 | 12 | Human Placenta |
| chr2: 119616133-119616826 | 13 | Human Placenta |
| chr2: 119914126-119916663 | 12 | Human Placenta |
| chr2: 124782252-124783255 | 11 | Human Placenta |
| chr2: 127413696-127414171 | 9 | Human Placenta |
| chr2: 127782613-127782829 | 14 | Human Placenta |
| chr2: 128421719-128422182 | 8 | Human Placenta |
| chr2: 130763483-130763764 | 13 | Human Placenta |
| chr2: 132182327-132183101 | 8 | Human Placenta |
| chr2: 139537692-139538650 | 10 | Human Placenta |
| chr2: 154727906-154728271 | 13 | Human Placenta |
| chr2: 154728944-154729328 | 12 | Human Placenta |
| chr2: 162279835-162280709 | 10 | Human Placenta |
| chr2: 162283581-162284677 | 13 | Human Placenta |
| chr2: 171671598-171671804 | 13 | Human Placenta |
| chr2: 171678546-171680358 | 10 | Human Placenta |
| chr2: 176931575-176932663 | 9 | Human Placenta |
| chr2: 176936246-176936809 | 9 | Human Placenta |
| chr2: 176944087-176948446 | 8 | Human Placenta |
| chr2: 176949993-176950336 | 13 | Human Placenta |
| chr2: 176956504-176956707 | 11 | Human Placenta |
| chr2: 177012371-177012675 | 15 | Human Placenta |
| chr2: 177016416-177016632 | 11 | Human Placenta |
| chr2: 177024501-177025692 | 12 | Human Placenta |
| chr2: 198029068-198029438 | 14 | Human Placenta |
| chr2: 200333687-200334172 | 12 | Human Placenta |
| chr2: 207506774-207507422 | 13 | Human Placenta |
| chr2: 220173870-220174283 | 14 | Human Placenta |
| chr2: 223159725-223160487 | 10 | Human Placenta |
| chr2: 223162946-223163912 | 13 | Human Placenta |
| chr2: 223167205-223167560 | 10 | Human Placenta |
| chr2: 223168653-223169008 | 12 | Human Placenta |
| chr2: 223176493-223177515 | 13 | Human Placenta |
| chr2: 233251361-233253414 | 12 | Human Placenta |
| chr2: 237068071-237068834 | 11 | Human Placenta |
| chr2: 238864315-238865170 | 12 | Human Placenta |
| chr2: 241459632-241460047 | 15 | Human Placenta |
| chr20: 690575-691099 | 13 | Human Placenta |
| chr20: 2539133-2539877 | 9 | Human Placenta |
| chr20: 2729997-2730797 | 8 | Human Placenta |
| chr20: 2780978-2781497 | 13 | Human Placenta |
| chr20: 5296266-5297798 | 11 | Human Placenta |
| chr20: 9496471-9496893 | 9 | Human Placenta |
| chr20: 10198135-10198984 | 10 | Human Placenta |
| chr20: 17206528-17206952 | 11 | Human Placenta |
| chr20: 17208550-17208756 | 13 | Human Placenta |
| chr20: 21376358-21378245 | 14 | Human Placenta |
| chr20: 21694472-21695344 | 11 | Human Placenta |
| chr20: 22548967-22549720 | 11 | Human Placenta |
| chr20: 25063838-25065525 | 9 | Human Placenta |
| chr20: 32856659-32857248 | 8 | Human Placenta |
| chr20: 36012595-36013439 | 9 | Human Placenta |
| chr20: 36226617-36226841 | 12 | Human Placenta |
| chr20: 41817475-41819212 | 8 | Human Placenta |
| chr20: 48184193-48184833 | 8 | Human Placenta |
| chr20: 57089460-57090237 | 13 | Human Placenta |
| chr20: 57426729-57427047 | 9 | Human Placenta |
| chr20: 61703526-61704022 | 11 | Human Placenta |
| chr21: 19617098-19617874 | 12 | Human Placenta |
| chr21: 34395128-34400245 | 11 | Human Placenta |
| chr21: 38076762-38077685 | 8 | Human Placenta |
| chr21: 38079941-38081833 | 8 | Human Placenta |
| chr21: 42218489-42219222 | 10 | Human Placenta |
| chr22: 19746155-19746369 | 11 | Human Placenta |
| chr22: 25081850-25082112 | 8 | Human Placenta |
| chr22: 37465056-37465331 | 11 | Human Placenta |
| chr22: 38379093-38379964 | 14 | Human Placenta |
| chr22: 39262338-39263211 | 8 | Human Placenta |
| chr22: 42685894-42686095 | 11 | Human Placenta |
| chr22: 44257942-44258612 | 9 | Human Placenta |
| chr22: 44287497-44288061 | 8 | Human Placenta |
| chr22: 48884884-48887043 | 11 | Human Placenta |
| chr22: 50496441-50497393 | 11 | Human Placenta |
| chr3: 238391-240140 | 9 | Human Placenta |
| chr3: 6904133-6904641 | 13 | Human Placenta |
| chr3: 9177691-9178189 | 9 | Human Placenta |
| chr3: 11034446-11035384 | 9 | Human Placenta |
| chr3: 12838471-12838782 | 8 | Human Placenta |
| chr3: 22413492-22414365 | 13 | Human Placenta |
| chr3: 26664104-26664796 | 12 | Human Placenta |
| chr3: 27771638-27771942 | 9 | Human Placenta |
| chr3: 32861141-32861429 | 10 | Human Placenta |
| chr3: 44063314-44063837 | 10 | Human Placenta |
| chr3: 44596535-44597018 | 8 | Human Placenta |
| chr3: 46618307-46618669 | 12 | Human Placenta |
| chr3: 62356119-62356378 | 14 | Human Placenta |
| chr3: 62356773-62357315 | 13 | Human Placenta |
| chr3: 62362610-62363082 | 14 | Human Placenta |
| chr3: 63263989-63264205 | 14 | Human Placenta |
| chr3: 64253533-64253819 | 10 | Human Placenta |
| chr3: 75667777-75669067 | 12 | Human Placenta |
| chr3: 75955759-75956308 | 9 | Human Placenta |
| chr3: 113160299-113160641 | 11 | Human Placenta |
| chr3: 121902742-121903645 | 12 | Human Placenta |
| chr3: 126113547-126113967 | 14 | Human Placenta |
| chr3: 127633993-127634588 | 9 | Human Placenta |
| chr3: 127794369-127796136 | 9 | Human Placenta |
| chr3: 128719865-128721245 | 8 | Human Placenta |
| chr3: 129693127-129694841 | 12 | Human Placenta |
| chr3: 133393118-133393657 | 12 | Human Placenta |
| chr3: 138656627-138659107 | 9 | Human Placenta |
| chr3: 147126988-147128999 | 14 | Human Placenta |
| chr3: 147138916-147139564 | 15 | Human Placenta |
| chr3: 147142181-147142391 | 12 | Human Placenta |
| chr3: 157812053-157812764 | 15 | Human Placenta |
| chr3: 170303532-170303768 | 14 | Human Placenta |
| chr3: 184056419-184056671 | 12 | Human Placenta |
| chr3: 185911344-185912228 | 9 | Human Placenta |
| chr3: 186078710-186080111 | 8 | Human Placenta |
| chr3: 192125821-192127994 | 8 | Human Placenta |
| chr4: 107146-107898 | 15 | Human Placenta |
| chr4: 206377-206892 | 15 | Human Placenta |
| chr4: 682724-683079 | 9 | Human Placenta |
| chr4: 961347-962155 | 14 | Human Placenta |
| chr4: 4859632-4860191 | 14 | Human Placenta |
| chr4: 5709985-5710495 | 13 | Human Placenta |
| chr4: 5891981-5892365 | 13 | Human Placenta |
| chr4: 5894071-5895116 | 13 | Human Placenta |
| chr4: 13524062-13526083 | 11 | Human Placenta |
| chr4: 15779998-15780729 | 9 | Human Placenta |
| chr4: 24801109-24801902 | 10 | Human Placenta |
| chr4: 41869174-41869459 | 12 | Human Placenta |
| chr4: 41875445-41875794 | 14 | Human Placenta |
| chr4: 41880224-41880500 | 13 | Human Placenta |
| chr4: 41882450-41882964 | 14 | Human Placenta |
| chr4: 46995128-46995872 | 14 | Human Placenta |
| chr4: 54975387-54976202 | 12 | Human Placenta |
| chr4: 57521621-57522703 | 8 | Human Placenta |
| chr4: 66535193-66535620 | 13 | Human Placenta |
| chr4: 81109887-81110460 | 13 | Human Placenta |
| chr4: 85403830-85404524 | 14 | Human Placenta |
| chr4: 85413997-85414874 | 11 | Human Placenta |
| chr4: 85422929-85423190 | 9 | Human Placenta |
| chr4: 93226348-93227007 | 10 | Human Placenta |
| chr4: 110222970-110224257 | 10 | Human Placenta |
| chr4: 111554965-111555504 | 13 | Human Placenta |
| chr4: 134069162-134070442 | 9 | Human Placenta |
| chr4: 140201064-140201449 | 8 | Human Placenta |
| chr4: 151504011-151505085 | 11 | Human Placenta |
| chr4: 154709512-154710827 | 10 | Human Placenta |
| chr4: 154712073-154712706 | 8 | Human Placenta |
| chr4: 154713537-154714240 | 11 | Human Placenta |
| chr4: 155663809-155664315 | 11 | Human Placenta |
| chr4: 156129168-156130209 | 11 | Human Placenta |
| chr4: 158143296-158144053 | 12 | Human Placenta |
| chr4: 169799086-169799625 | 9 | Human Placenta |
| chr4: 174422024-174422443 | 12 | Human Placenta |
| chr4: 174427891-174428192 | 11 | Human Placenta |
| chr4: 174437914-174438346 | 12 | Human Placenta |
| chr4: 174439812-174440249 | 13 | Human Placenta |
| chr4: 174448333-174448845 | 12 | Human Placenta |
| chr4: 174450046-174451469 | 11 | Human Placenta |
| chr4: 174451828-174452962 | 11 | Human Placenta |
| chr4: 174459200-174460054 | 8 | Human Placenta |
| chr4: 185937242-185937750 | 11 | Human Placenta |
| chr4: 187219320-187219745 | 8 | Human Placenta |
| chr4: 188916605-188916876 | 14 | Human Placenta |
| chr4: 190938106-190938848 | 13 | Human Placenta |
| chr4: 190939801-190940591 | 12 | Human Placenta |
| chr5: 1874907-1879032 | 12 | Human Placenta |
| chr5: 2738953-2741237 | 10 | Human Placenta |
| chr5: 3590644-3592000 | 10 | Human Placenta |
| chr5: 3594467-3603054 | 12 | Human Placenta |
| chr5: 11384681-11385521 | 9 | Human Placenta |
| chr5: 31193952-31194419 | 8 | Human Placenta |
| chr5: 45695394-45696510 | 12 | Human Placenta |
| chr5: 50685453-50686148 | 13 | Human Placenta |
| chr5: 54519054-54519628 | 9 | Human Placenta |
| chr5: 63255044-63255407 | 13 | Human Placenta |
| chr5: 72526203-72526497 | 12 | Human Placenta |
| chr5: 72594147-72595808 | 9 | Human Placenta |
| chr5: 72676120-72678421 | 9 | Human Placenta |
| chr5: 76923887-76924502 | 15 | Human Placenta |
| chr5: 76936126-76936984 | 8 | Human Placenta |
| chr5: 77140542-77140914 | 13 | Human Placenta |
| chr5: 77146998-77147785 | 13 | Human Placenta |
| chr5: 77253832-77254049 | 13 | Human Placenta |
| chr5: 77268350-77268787 | 8 | Human Placenta |
| chr5: 87968635-87968907 | 13 | Human Placenta |
| chr5: 87980878-87981272 | 12 | Human Placenta |
| chr5: 87985470-87985810 | 11 | Human Placenta |
| chr5: 88185224-88185589 | 9 | Human Placenta |
| chr5: 115697134-115697589 | 8 | Human Placenta |
| chr5: 122430676-122431443 | 9 | Human Placenta |
| chr5: 134385967-134386370 | 13 | Human Placenta |
| chr5: 140346105-140346931 | 8 | Human Placenta |
| chr5: 140787447-140788044 | 14 | Human Placenta |
| chr5: 140864527-140864748 | 10 | Human Placenta |
| chr5: 146888750-146889840 | 8 | Human Placenta |
| chr5: 148033472-148034080 | 10 | Human Placenta |
| chr5: 158478378-158478630 | 8 | Human Placenta |
| chr5: 159399004-159399928 | 8 | Human Placenta |
| chr5: 170735169-170739863 | 10 | Human Placenta |
| chr5: 170741603-170742751 | 13 | Human Placenta |
| chr5: 170743178-170744107 | 11 | Human Placenta |
| chr5: 172110282-172111166 | 12 | Human Placenta |
| chr5: 172659049-172660277 | 13 | Human Placenta |
| chr5: 172660720-172661133 | 12 | Human Placenta |
| chr5: 172661486-172662228 | 9 | Human Placenta |
| chr5: 172672311-172672971 | 13 | Human Placenta |
| chr5: 174158680-174159729 | 11 | Human Placenta |
| chr5: 175085004-175085756 | 11 | Human Placenta |
| chr5: 178421225-178422337 | 14 | Human Placenta |
| chr5: 180486154-180486892 | 9 | Human Placenta |
| chr6: 1378445-1379318 | 12 | Human Placenta |
| chr6: 1393049-1394170 | 12 | Human Placenta |
| chr6: 1619093-1621094 | 9 | Human Placenta |
| chr6: 4079052-4079443 | 12 | Human Placenta |
| chr6: 5999149-5999787 | 10 | Human Placenta |
| chr6: 10381558-10382354 | 11 | Human Placenta |
| chr6: 10881846-10882051 | 14 | Human Placenta |
| chr6: 26614013-26614851 | 11 | Human Placenta |
| chr6: 27228100-27228364 | 13 | Human Placenta |
| chr6: 29595298-29595795 | 12 | Human Placenta |
| chr6: 30095173-30095610 | 14 | Human Placenta |
| chr6: 30139718-30140263 | 11 | Human Placenta |
| chr6: 33048416-33048814 | 12 | Human Placenta |
| chr6: 35479388-35479678 | 15 | Human Placenta |
| chr6: 37616722-37617179 | 15 | Human Placenta |
| chr6: 38682949-38683265 | 13 | Human Placenta |
| chr6: 41528266-41528900 | 10 | Human Placenta |
| chr6: 42145847-42146053 | 11 | Human Placenta |
| chr6: 42879279-42879623 | 14 | Human Placenta |
| chr6: 50787286-50788091 | 12 | Human Placenta |
| chr6: 50810642-50810994 | 13 | Human Placenta |
| chr6: 50813314-50813699 | 12 | Human Placenta |
| chr6: 50818180-50818431 | 12 | Human Placenta |
| chr6: 70992040-70992912 | 10 | Human Placenta |
| chr6: 72298274-72298528 | 11 | Human Placenta |
| chr6: 78172231-78174088 | 13 | Human Placenta |
| chr6: 85472702-85474132 | 11 | Human Placenta |
| chr6: 99290279-99290771 | 11 | Human Placenta |
| chr6: 100038655-100039477 | 9 | Human Placenta |
| chr6: 100897080-100897621 | 15 | Human Placenta |
| chr6: 100903491-100903713 | 11 | Human Placenta |
| chr6: 100905444-100905697 | 14 | Human Placenta |
| chr6: 100905952-100906686 | 12 | Human Placenta |
| chr6: 100914946-100915245 | 9 | Human Placenta |
| chr6: 106429111-106429772 | 15 | Human Placenta |
| chr6: 106433984-106434459 | 9 | Human Placenta |
| chr6: 108495654-108495986 | 14 | Human Placenta |
| chr6: 110299365-110301267 | 8 | Human Placenta |
| chr6: 117869097-117869530 | 8 | Human Placenta |
| chr6: 127441553-127441760 | 8 | Human Placenta |
| chr6: 137809342-137810204 | 13 | Human Placenta |
| chr6: 137816474-137817223 | 9 | Human Placenta |
| chr6: 150335525-150336278 | 12 | Human Placenta |
| chr6: 150358872-150359394 | 12 | Human Placenta |
| chr6: 154360586-154361008 | 10 | Human Placenta |
| chr6: 161188084-161188639 | 8 | Human Placenta |
| chr6: 166579973-166583423 | 12 | Human Placenta |
| chr6: 166666837-166667541 | 9 | Human Placenta |
| chr6: 168841438-168841699 | 8 | Human Placenta |
| chr6: 170732119-170732442 | 14 | Human Placenta |
| chr7: 751712-752150 | 13 | Human Placenta |
| chr7: 12151220-12151559 | 11 | Human Placenta |
| chr7: 19184818-19185033 | 14 | Human Placenta |
| chr7: 23287221-23287508 | 11 | Human Placenta |
| chr7: 27134097-27134303 | 9 | Human Placenta |
| chr7: 27147589-27148389 | 13 | Human Placenta |
| chr7: 27198182-27198514 | 9 | Human Placenta |
| chr7: 27203915-27206462 | 9 | Human Placenta |
| chr7: 27260101-27260467 | 10 | Human Placenta |
| chr7: 27291119-27292197 | 10 | Human Placenta |
| chr7: 32110063-32110910 | 13 | Human Placenta |
| chr7: 35296921-35298218 | 10 | Human Placenta |
| chr7: 42267546-42267823 | 10 | Human Placenta |
| chr7: 43152020-43153340 | 8 | Human Placenta |
| chr7: 53286851-53287192 | 10 | Human Placenta |
| chr7: 54612324-54612558 | 14 | Human Placenta |
| chr7: 70596228-70598382 | 12 | Human Placenta |
| chr7: 71800757-71802768 | 10 | Human Placenta |
| chr7: 72838383-72838815 | 11 | Human Placenta |
| chr7: 73894815-73895110 | 8 | Human Placenta |
| chr7: 89747892-89749036 | 9 | Human Placenta |
| chr7: 97361132-97363018 | 10 | Human Placenta |
| chr7: 100075303-100075551 | 8 | Human Placenta |
| chr7: 100817759-100817975 | 9 | Human Placenta |
| chr7: 100823307-100823701 | 11 | Human Placenta |
| chr7: 101005899-101007443 | 9 | Human Placenta |
| chr7: 103085710-103086132 | 16 | Human Placenta |
| chr7: 103968783-103969959 | 9 | Human Placenta |
| chr7: 121940006-121940648 | 14 | Human Placenta |
| chr7: 121950249-121950927 | 11 | Human Placenta |
| chr7: 121956543-121957341 | 11 | Human Placenta |
| chr7: 124404174-124404432 | 11 | Human Placenta |
| chr7: 127990926-127992616 | 11 | Human Placenta |
| chr7: 128555329-128556650 | 12 | Human Placenta |
| chr7: 129422997-129423355 | 12 | Human Placenta |
| chr7: 142494563-142495248 | 9 | Human Placenta |
| chr7: 143582125-143582610 | 10 | Human Placenta |
| chr7: 149389654-149389976 | 14 | Human Placenta |
| chr7: 149744402-149746469 | 13 | Human Placenta |
| chr7: 152621916-152622149 | 11 | Human Placenta |
| chr7: 153748407-153750444 | 14 | Human Placenta |
| chr7: 154001964-154002281 | 13 | Human Placenta |
| chr7: 155164557-155167854 | 11 | Human Placenta |
| chr7: 155174128-155175248 | 9 | Human Placenta |
| chr7: 155241323-155243757 | 11 | Human Placenta |
| chr7: 155258827-155261403 | 8 | Human Placenta |
| chr7: 155302253-155303158 | 8 | Human Placenta |
| chr7: 156409023-156409294 | 8 | Human Placenta |
| chr7: 156409577-156409865 | 9 | Human Placenta |
| chr7: 156801418-156801632 | 8 | Human Placenta |
| chr7: 156871054-156871297 | 11 | Human Placenta |
| chr7: 158936507-158938492 | 14 | Human Placenta |
| chr8: 4848968-4852635 | 8 | Human Placenta |
| chr8: 9760750-9761643 | 12 | Human Placenta |
| chr8: 9762661-9764748 | 12 | Human Placenta |
| chr8: 11536767-11538961 | 11 | Human Placenta |
| chr8: 11557852-11558252 | 12 | Human Placenta |
| chr8: 11565217-11567212 | 14 | Human Placenta |
| chr8: 21644908-21647845 | 9 | Human Placenta |
| chr8: 23562475-23565175 | 13 | Human Placenta |
| chr8: 23567180-23567678 | 14 | Human Placenta |
| chr8: 24812946-24814299 | 10 | Human Placenta |
| chr8: 26721642-26724566 | 11 | Human Placenta |
| chr8: 37822486-37824008 | 12 | Human Placenta |
| chr8: 41424341-41425300 | 12 | Human Placenta |
| chr8: 49468683-49468959 | 11 | Human Placenta |
| chr8: 50822270-50822860 | 12 | Human Placenta |
| chr8: 53851701-53854426 | 9 | Human Placenta |
| chr8: 55370170-55372525 | 13 | Human Placenta |
| chr8: 55378928-55380186 | 13 | Human Placenta |
| chr8: 57358126-57359415 | 14 | Human Placenta |
| chr8: 65281903-65283043 | 14 | Human Placenta |
| chr8: 65286067-65286659 | 9 | Human Placenta |
| chr8: 65290108-65290946 | 14 | Human Placenta |
| chr8: 68864584-68864946 | 10 | Human Placenta |
| chr8: 72468560-72469561 | 13 | Human Placenta |
| chr8: 85096759-85097247 | 13 | Human Placenta |
| chr8: 86350765-86351196 | 12 | Human Placenta |
| chr8: 87081653-87082046 | 10 | Human Placenta |
| chr8: 97169731-97170432 | 11 | Human Placenta |
| chr8: 97171805-97172022 | 14 | Human Placenta |
| chr8: 98289604-98290404 | 11 | Human Placenta |
| chr8: 99960497-99961438 | 11 | Human Placenta |
| chr8: 99984584-99985072 | 10 | Human Placenta |
| chr8: 99985733-99986983 | 14 | Human Placenta |
| chr8: 101117922-101118693 | 13 | Human Placenta |
| chr8: 130995921-130996149 | 12 | Human Placenta |
| chr8: 132052203-132054749 | 13 | Human Placenta |
| chr8: 139508795-139509774 | 12 | Human Placenta |
| chr8: 142528185-142529029 | 12 | Human Placenta |
| chr8: 145103285-145108027 | 12 | Human Placenta |
| chr8: 145925410-145926101 | 13 | Human Placenta |
| chr9: 969529-973276 | 12 | Human Placenta |
| chr9: 16726859-16727273 | 8 | Human Placenta |
| chr9: 19788215-19789288 | 12 | Human Placenta |
| chr9: 23820691-23822135 | 8 | Human Placenta |
| chr9: 23850910-23851522 | 12 | Human Placenta |
| chr9: 32782936-32783625 | 11 | Human Placenta |
| chr9: 36739534-36739782 | 14 | Human Placenta |
| chr9: 37002489-37002957 | 14 | Human Placenta |
| chr9: 77112712-77113583 | 9 | Human Placenta |
| chr9: 77113709-77113927 | 13 | Human Placenta |
| chr9: 79633326-79636030 | 9 | Human Placenta |
| chr9: 79637814-79638169 | 13 | Human Placenta |
| chr9: 91792662-91793611 | 10 | Human Placenta |
| chr9: 96108466-96108992 | 15 | Human Placenta |
| chr9: 96710811-96711717 | 13 | Human Placenta |
| chr9: 98111364-98112362 | 12 | Human Placenta |
| chr9: 100610696-100611517 | 12 | Human Placenta |
| chr9: 100619984-100620192 | 11 | Human Placenta |
| chr9: 104499849-104501076 | 8 | Human Placenta |
| chr9: 115822071-115823416 | 13 | Human Placenta |
| chr9: 120507227-120507642 | 9 | Human Placenta |
| chr9: 123656750-123656972 | 15 | Human Placenta |
| chr9: 134429866-134430491 | 10 | Human Placenta |
| chr9: 136294738-136295236 | 15 | Human Placenta |
| chr9: 137967110-137967727 | 10 | Human Placenta |
| chr9: 139715663-139716441 | 13 | Human Placenta |

TABLE II

| chr1: 1072370-1072847 | chr11: 65190825-65191058 | chr16: 72821141-72821592 |
|---|---|---|
| chr1: 10895896-10896117 | chr11: 65222491-65222750 | chr16: 73099813-73100791 |
| chr1: 109203594-109204378 | chr11: 65341621-65342501 | chr16: 743925-745943 |
| chr1: 1093212-1093476 | chr11: 65343330-65343849 | chr16: 78079753-78080166 |
| chr1: 110185962-110186164 | chr11: 65553750-65555573 | chr16: 80574742-80575090 |
| chr1: 110626529-110627484 | chr11: 65779312-65779767 | chr16: 80965953-80966478 |
| chr1: 110880395-110880624 | chr11: 66034752-66035054 | chr16: 84029457-84029710 |
| chr1: 111505882-111507007 | chr11: 66035217-66035447 | chr16: 84328520-84328720 |
| [symbol] 38-111747303 | chr11: 66049751-66050229 | chr16: 84346477-84346931 |
| chr1: 113044411-113044992 | chr11: 66314208-66314455 | chr16: 84401958-84402497 |
| chr1: 113392143-113392807 | chr11: 66335576-66336151 | chr16: 85171020-85171323 |
| chr1: 113497987-113498206 | chr11: 67232299-67232558 | chr16: 85783863-85785131 |
| chr1: 1141671-1142150 | chr11: 67770427-67771629 | chr16: 85863382-85863601 |
| chr1: 11538670-11540342 | chr11: 67806252-67806611 | chr16: 85932122-85932942 |
| chr1: 116694665-116694983 | chr11: 68611251-68611807 | chr16: 86546360-86546632 |
| chr1: 116710838-116711260 | chr11: 69258150-69258544 | chr16: 87902455-87903460 |
| chr1: 11710460-11710788 | chr11: 69924339-69925197 | chr16: 88292764-88293010 |
| chr1: 11779567-11780016 | chr11: 705795-706534 | chr16: 88716990-88717606 |
| chr1: 118727817-118728097 | chr11: 70962174-70964161 | chr16: 88803803-88804112 |
| chr1: 120835962-120839391 | chr11: 71954817-71955659 | chr16: 88850205-88850537 |
| chr1: 12655927-12656248 | chr11: 720562-721369 | chr16: 89070647-89070904 |
| chr1: 1362955-1363299 | chr11: 72301303-72301746 | chr16: 89267824-89268087 |
| chr1: 1370768-1371449 | chr11: 72463093-72463717 | chr16: 89268493-89268865 |
| chr1: 13839506-13840613 | chr11: 72492282-72492644 | chr16: 89323281-89323661 |
| chr1: 13909607-13909842 | chr11: 74022429-74022703 | chr16: 89632593-89632799 |
| chr1: 14026482-14027200 | chr11: 75236190-75237781 | chr16: 90014251-90014613 |
| chr1: 14219351-14219737 | chr11: 75917272-75917926 | chr17: 10632790-10633490 |
| chr1: 146556313-146556676 | chr11: 77122737-77123088 | chr17: 11501632-11502328 |
| chr1: 14924611-14925993 | chr11: 78673008-78673213 | chr17: 1163342-1163773 |
| chr1: 149605515-149605903 | chr11: 789872-790133 | chr17: 12692738-12693690 |
| chr1: 150254366-150254637 | chr11: 8102359-8102913 | chr17: 1390457-1390786 |
| chr1: 150266477-150266689 | chr11: 826942-827625 | chr17: 1395120-1395372 |
| chr1: 151300523-151300724 | chr11: 8284103-8285032 | chr17: 14212364-14212788 |
| chr1: 151445872-151446142 | chr11: 86382696-86383586 | chr17: 15244706-15245126 |
| chr1: 151693992-151694282 | chr11: 87908244-87908614 | chr17: 15466360-15466843 |
| chr1: 151812254-151812525 | chr11: 9025096-9026315 | chr17: 1546743-1547324 |
| chr1: 151966633-151966893 | chr11: 93583375-93583717 | chr17: 1551731-1553249 |
| chr1: 152079998-152081705 | chr11: 94473536-94474338 | chr17: 15847758-15849513 |
| chr1: 154298206-154298544 | chr11: 94501367-94502696 | chr17: 16283928-16284768 |
| chr1: 154732823-154733436 | chr11: 9634970-9636065 | chr17: 17685017-17687240 |
| chr1: 154971871-154972404 | chr11: 9779593-9780470 | chr17: 18965478-18965728 |
| chr1: 155043413-155043922 | chr11: 98891544-98891821 | chr17: 2627241-2628302 |
| chr1: 155830196-155830489 | chr12: 103350090-103350422 | chr17: 26578273-26578682 |
| chr1: 156051240-156051461 | chr12: 103351580-103352695 | chr17: 26645291-26645614 |
| chr1: 156616554-156616946 | chr12: 103359249-103359629 | chr17: 26698360-26699557 |
| chr1: 156646293-156647260 | chr12: 104850254-104852395 | chr17: 26711384-26712311 |
| chr1: 156814882-156815792 | chr12: 105478090-105478517 | chr17: 27038085-27038919 |
| chr1: 156893520-156894232 | chr12: 106532107-106533696 | chr17: 27332269-27333188 |
| chr1: 157963541-157963947 | chr12: 107711604-107714107 | chr17: 27503599-27504014 |
| [symbol] 89-158119704 | chr12: 108297427-108297743 | chr17: 27942533-27945388 |
| chr1: 159141203-159141718 | chr12: 109162409-109162722 | chr17: 27949430-27950277 |
| chr1: 15929824-15930289 | chr12: 109729573-109729826 | chr17: 29298047-29298606 |
| chr1: 160040129-160040668 | chr12: 110150048-110150262 | chr17: 29718231-29719291 |
| chr1: 16085148-16085862 | chr12: 110156268-110156496 | chr17: 29814615-29815662 |
| chr1: 161228478-161229028 | chr12: 111471961-111473546 | chr17: 30593199-30594033 |
| chr1: 162760251-162760722 | chr12: 112204499-112204979 | chr17: 30845904-30846702 |
| chr1: 162792177-162792574 | chr12: 115104849-115105548 | chr17: 32953154-32953801 |
| chr1: 16543684-16544307 | chr12: 115120775-115122945 | chr17: 33787402-33787845 |
| chr1: 166134259-166136448 | chr12: 115135926-115136350 | chr17: 33814235-33814947 |
| chr1: 167789397-167789647 | chr12: 115889598-115889995 | chr17: 34091137-34091919 |
| chr1: 17033769-17034728 | chr12: 116354788-116355187 | chr17: 3438842-3439046 |
| chr1: 171810468-171811325 | chr12: 116946196-116946607 | chr17: 35060323-35060692 |
| chr1: 179555402-179555770 | chr12: 117316390-117317611 | chr17: 35303285-35303572 |
| chr1: 180881317-180882592 | chr12: 117536291-117537421 | chr17: 36102034-36104766 |
| chr1: 182584178-182584545 | chr12: 120031495-120033212 | chr17: 36105335-36105583 |
| chr1: 184633224-184633663 | chr12: 120799373-120799912 | chr17: 36575500-36575782 |
| chr1: 1875618-1875877 | chr12: 122277302-122277539 | chr17: 36584421-36585453 |
| chr1: 18971730-18972097 | chr12: 122667649-122668038 | chr17: 36728634-36729284 |
| chr1: 19970256-19971923 | chr12: 123380334-123380894 | chr17: 37365987-37366539 |
| chr1: 200860077-200860576 | chr12: 126018101-126018365 | chr17: 37856449-37856891 |
| chr1: 200992283-200992839 | chr12: 128850550-128850755 | chr17: 38020382-38020645 |
| chr1: 201368561-201369032 | chr12: 129787736-129788160 | chr17: 38347534-38347765 |
| chr1: 201450881-201451105 | chr12: 130526916-130527117 | chr17: 38497528-38498963 |
| chr1: 201475886-201476516 | chr12: 13152820-13153084 | chr17: 38501397-38501839 |
| chr1: 201708788-201709429 | chr12: 132312440-132315739 | chr17: 39683909-39684599 |
| chr1: 202936046-202936252 | chr12: 132689881-132690197 | chr17: 39705046-39705332 |
| chr1: 203456785-203457059 | chr12: 132690340-132690571 | chr17: 40250273-40250591 |
| chr1: 203598472-203598853 | chr12: 133463808-133464858 | chr17: 40332598-40333471 |
| chr1: 204159599-204159833 | chr12: 14927292-14928023 | chr17: 40440189-40441014 |
| chr1: 204797611-204797930 | chr12: 175667-176400 | chr17: 40805675-40805957 |
| chr1: 20512361-20512797 | chr12: 1770702-1771476 | chr17: 40912817-40913553 |
| chr1: 205537752-205538443 | chr12: 1905278-1906765 | chr17: 40932330-40933299 |
| chr1: 206223538-206224028 | chr12: 20521617-20523122 | chr17: 41723220-41723826 |
| chr1: 2064629-2064855 | chr12: 21680409-21680982 | chr17: 41791111-41791476 |
| chr1: 206730398-206730908 | chr12: 21810489-21810766 | chr17: 41984149-41985012 |
| chr1: 20810463-20813511 | chr12: 22486836-22488666 | chr17: 42015422-42015707 |
| chr1: 209848444-209849428 | chr12: 24714957-24716243 | chr17: 42015844-42016069 |
| chr1: 209979317-209979666 | chr12: 26348261-26349130 | chr17: 42030174-42030941 |
| chr1: 210465710-210466212 | chr12: 2800140-2801062 | chr17: 42061047-42061643 |
| chr1: 211306668-211307675 | chr12: 28127891-28128575 | chr17: 42082028-42084972 |
| [symbol] 62-211689104 | chr12: 29935996-29937433 | chr17: 42091713-42091948 |
| chr1: 213123648-213125092 | chr12: 3862069-3862606 | chr17: 42092144-42092432 |
| chr1: 214161198-214161415 | chr12: 4273820-4274491 | chr17: 42287693-42288392 |
| chr1: 2144200-2144497 | chr12: 4378367-4382222 | chr17: 42392324-42393079 |
| chr1: 215256052-215256636 | chr12: 4383194-4384405 | chr17: 42402788-42403266 |
| chr1: 219347110-219347572 | chr12: 49318487-49319476 | chr17: 44026528-44026738 |
| chr1: 220960017-220960603 | chr12: 49363665-49364443 | chr17: 44848309-44849912 |
| chr1: 2222199-2222569 | chr12: 49390618-49392441 | chr17: 45400875-45401440 |
| chr1: 225117221-225117781 | chr12: 49487964-49488202 | chr17: 45928212-45928710 |
| chr1: 226270724-226271841 | chr12: 49688874-49691360 | chr17: 46089637-46089851 |
| chr1: 22668639-22668862 | chr12: 49735720-49736875 | chr17: 46114574-46115059 |
| chr1: 226736355-226737412 | chr12: 50297581-50297988 | chr17: 46507345-46507778 |
| chr1: 227729516-227730492 | chr12: 50349080-50349525 | chr17: 46655216-46655604 |
| chr1: 228565950-228567121 | chr12: 51785280-51785821 | chr17: 46687528-46688730 |
| chr1: 230561104-230562702 | chr12: 51818461-51819166 | chr17: 46710813-46711419 |
| chr1: 231175063-231176317 | chr12: 52444554-52445421 | chr17: 46719361-46720234 |
| chr1: 231176786-231177009 | chr12: 52545938-52546363 | chr17: 46723732-46724383 |
| chr1: 232941055-232941707 | chr12: 52701963-52702560 | chr17: 46755566-46756006 |
| chr1: 233749374-233750314 | chr12: 53267860-53268290 | chr17: 46827436-46827641 |
| chr1: 236687072-236687608 | chr12: 53273232-53273498 | chr17: 47209812-47210740 |
| chr1: 23750509-23751663 | chr12: 53297443-53297824 | chr17: 47572346-47575316 |
| chr1: 23884843-23885087 | chr12: 53441385-53441706 | chr17: 47647377-47647660 |
| chr1: 240254960-240257063 | chr12: 53448009-53448406 | chr17: 47967874-47968409 |
| chr1: 244012713-244013245 | chr12: 53613717-53615103 | chr17: 48619112-48619794 |
| chr1: 244213398-244213619 | chr12: 53718633-53719778 | chr17: 49021857-49022279 |
| chr1: 2460761-2462010 | chr12: 54332806-54333731 | chr17: 4981358-4981979 |
| chr1: 24648203-24648985 | chr12: 54343623-54343848 | chr17: 52977867-52978307 |
| chr1: 24739858-24740262 | chr12: 54346778-54347101 | chr17: 53315619-53316198 |
| chr1: 2477564-2478363 | chr12: 54387826-54388732 | chr17: 53342199-53343061 |
| chr1: 26490523-26491015 | chr12: 54389114-54389520 | chr17: 54674159-54674366 |
| chr1: 26686517-26687281 | chr12: 54393375-54394648 | chr17: 54910497-54912470 |
| chr1: 27338880-27339441 | chr12: 54399413-54399654 | chr17: 55939089-55939591 |
| chr1: 27854417-27854963 | chr12: 54399907-54400495 | chr17: 56832962-56833986 |
| chr1: 27894928-27895524 | chr12: 54408427-54408713 | chr17: 58228062-58228361 |
| chr1: 27960568-27961023 | chr12: 54784901-54785238 | chr17: 58498712-58499332 |
| chr1: 27986306-27986808 | chr12: 5541211-5542973 | chr17: 59473061-59483266 |
| chr1: 29101791-29102069 | chr12: 56522944-56523285 | chr17: 59539363-59539834 |
| chr1: 2929156-2929376 | chr12: 56881402-56882702 | chr17: 5973316-5974449 |
| chr1: 31158010-31158261 | chr12: 58013172-58013652 | chr17: 60704543-60705943 |
| chr1: 31380845-31381078 | chr12: 58021295-58022037 | chr17: 60729646-60730269 |
| chr1: 32169538-32169869 | chr12: 58258987-58259496 | chr17: 60885409-60885857 |
| [symbol] 2-32180487 | chr12: 59989652-59990507 | chr17: 61523001-61524470 |
| chr1: 32226147-32226535 | chr12: 63025589-63026213 | chr17: 61615393-61616035 |
| chr1: 32237828-32238661 | chr12: 6419604-6420024 | chr17: 6460072-6460302 |
| chr1: 3239916-3240261 | chr12: 6472661-6473322 | chr17: 64961008-64962321 |
| chr1: 32410189-32410630 | chr12: 6492890-6493522 | chr17: 68164914-68165720 |
| chr1: 32892429-32892835 | chr12: 65672392-65673250 | chr17: 6945390-6947521 |
| chr1: 3310103-3311035 | chr12: 6664426-6665336 | chr17: 7108305-7108654 |
| chr1: 33219428-33220028 | chr12: 71003625-71004057 | chr17: 71639029-71641670 |
| chr1: 33358470-33359449 | chr12: 8123347-8123561 | chr17: 7164286-7166245 |
| chr1: 33438459-33438979 | chr12: 81330609-81331514 | chr17: 71897971-71898420 |
| chr1: 33938026-33938328 | chr12: 82152321-82152674 | chr17: 72270302-72270512 |
| chr1: 3447450-3447950 | chr12: 8849964-8851403 | chr17: 72352828-72353798 |
| chr1: 35331704-35332409 | chr12: 96883287-96883737 | chr17: 72442928-72443194 |
| chr1: 35350879-35351854 | chr12: 98850691-98851002 | chr17: 72449983-72450617 |
| chr1: 35394748-35396206 | chr12: 99288223-99289374 | chr17: 72667196-72667710 |
| chr1: 36042433-36043444 | chr13: 100631753-100635526 | chr17: 72732849-72733545 |
| chr1: 3662964-3664085 | chr13: 102068117-102069258 | chr17: 72848167-72848901 |
| chr1: 36771831-36773009 | chr13: 107186469-107189024 | chr17: 72889350-72890060 |
| chr1: 3688554-3689684 | chr13: 110958892-110960590 | chr17: 72931730-72932601 |
| chr1: 37498378-37500624 | chr13: 110960925-110961143 | chr17: 73030677-73031160 |
| chr1: 38229839-38230888 | chr13: 113548644-113549127 | chr17: 73083867-73084495 |
| chr1: 41847265-41849204 | chr13: 113597554-113598303 | chr17: 73520957-73522540 |
| chr1: 43832815-43833073 | chr13: 113622738-113623660 | chr17: 74496993-74497632 |
| chr1: 44401758-44402423 | chr13: 113807289-113807865 | chr17: 74706466-74707067 |
| chr1: 44871110-44874047 | chr13: 114017867-114018463 | chr17: 75524590-75525152 |
| chr1: 44883137-44884272 | chr13: 20175967-20176766 | chr17: 7608377-7608956 |
| chr1: 46767426-46769036 | chr13: 20702852-20703122 | chr17: 76127522-76128406 |
| chr1: 46859725-46860291 | chr13: 20806017-20806867 | chr17: 77093292-77093805 |
| chr1: 46913787-46914343 | chr13: 23489631-23490058 | chr17: 77783810-77789842 |
| chr1: 47489227-47489633 | chr13: 26760422-26760892 | chr17: 78977614-78978447 |
| chr1: 47690981-47691727 | chr13: 28491489-28492518 | chr17: 79366807-79374742 |
| chr1: 47915640-47915952 | chr13: 28501860-28502090 | chr17: 79448219-79448827 |
| chr1: 47998900-47999517 | chr13: 28542222-28543544 | chr17: 79485600-79486913 |
| chr1: 48058794-48059230 | chr13: 28554428-28555065 | chr17: 79614851-79615559 |
| chr1: 48190757-48190992 | chr13: 29105412-29105676 | chr17: 79917287-79920022 |
| chr1: 48449871-48450144 | chr13: 29292671-29293329 | chr17: 80329067-80330208 |
| chr1: 48462132-48462976 | chr13: 30995686-30995947 | chr17: 8054551-8055835 |
| chr1: 48937305-48937683 | chr13: 31019861-31020137 | chr17: 9142981-9143839 |
| chr1: 49242372-49242810 | chr13: 31377146-31377493 | chr18: 10131854-10132080 |
| chr1: 50513645-50514320 | chr13: 36705041-36705707 | chr18: 12271658-12271920 |
| chr1: 50798668-50799536 | chr13: 36871668-36872059 | chr18: 12407716-12408242 |
| [symbol] 8-53387523 | chr13: 36919738-36921004 | chr18: 13136597-13137564 |
| chr1: 55446088-55446846 | chr13: 37005582-37006453 | chr18: 13824002-13824215 |
| chr1: 57110664-57111337 | chr13: 37247950-37248463 | chr18: 14132001-14132482 |
| chr1: 57887964-57890637 | chr13: 43597505-43597856 | chr18: 14430639-14431567 |
| chr1: 59280952-59281194 | chr13: 44359859-44361696 | chr18: 19928417-19929329 |
| chr1: 60280625-60281048 | chr13: 44453208-44454295 | chr18: 20139485-20140246 |
| chr1: 6086245-6086494 | chr13: 45885755-45886103 | chr18: 21199432-21199798 |
| chr1: 61508643-61509282 | chr13: 50070023-50070719 | chr18: 21269270-21270349 |
| chr1: 61519353-61519971 | chr13: 53174029-53174674 | chr18: 22006311-22007007 |
| chr1: 6208717-6209039 | chr13: 53424942-53425995 | chr18: 24443201-24443458 |
| chr1: 6241032-6241251 | chr13: 67804594-67805459 | chr18: 25755415-25758175 |
| chr1: 6265826-6266778 | chr13: 88323570-88324640 | chr18: 2846566-2848175 |
| chr1: 6301696-6302856 | chr13: 95201561-95202293 | chr18: 28681321-28682455 |
| chr1: 6484504-6485327 | chr13: 95354190-95355184 | chr18: 2905951-2907348 |
| chr1: 6507208-6509186 | chr13: 95619884-95620977 | chr18: 29077553-29078526 |
| chr1: 6545144-6545559 | chr13: 96204692-96205496 | chr18: 31802359-31803792 |
| chr1: 65468273-65468828 | chr13: 98794399-98796241 | chr18: 31804481-31804875 |
| chr1: 68696640-68697628 | chr14: 100204041-100204262 | chr18: 34823799-34824017 |
| chr1: 70032968-70034495 | chr14: 100625320-100626461 | chr18: 3498942-3499358 |
| chr1: 72748472-72749736 | chr14: 100680270-100680947 | chr18: 42258984-42260795 |
| chr1: 76080455-76080808 | chr14: 101012841-101013117 | chr18: 43355319-43355698 |
| chr1: 76540148-76540653 | chr14: 101121009-101121365 | chr18: 43913346-43914477 |
| chr1: 77333112-77334534 | chr14: 102247661-102248279 | chr18: 44526867-44527137 |
| chr1: 8002409-8002699 | chr14: 103010785-103011625 | chr18: 45662315-45663520 |
| chr1: 8013994-8014651 | chr14: 103389017-103390248 | chr18: 47087076-47087446 |
| chr1: 805199-805628 | chr14: 103394398-103397070 | chr18: 47087880-47088528 |
| chr1: 82265999-82269048 | chr14: 103589354-103590246 | chr18: 48085839-48087589 |
| chr1: 8277196-8277822 | chr14: 103739579-103740892 | chr18: 5237724-5238674 |
| chr1: 84326268-84326838 | chr14: 105293561-105293879 | chr18: 52495605-52496007 |
| chr1: 85462586-85463435 | chr14: 105309691-105310500 | chr18: 52626518-52626849 |
| chr1: 87617154-87617417 | chr14: 105330515-105332186 | chr18: 53447075-53447842 |
| chr1: 87617693-87617986 | chr14: 105512056-105512307 | chr18: 5542848-5543054 |
| chr1: 90308840-90309606 | chr14: 105662778-105663403 | chr18: 55862654-55862873 |
| chr1: 9258566-9258956 | chr14: 105952604-105954296 | chr18: 56932714-56932994 |
| chr1: 94702691-94703344 | chr14: 21100839-21101043 | chr18: 56934834-56936827 |
| chr1: 95285603-95286319 | chr14: 21537083-21537355 | chr18: 5890293-5891682 |
| chr1: 9749296-9750228 | chr14: 21539712-21540045 | chr18: 59992070-59993556 |
| chr1: 999679-999911 | chr14: 23355739-23356402 | chr18: 63417776-63418640 |
| chr10: 100227439-100227832 | chr14: 24457865-24458100 | chr18: 65183238-65184014 |
| chr10: 101293016-101293238 | chr14: 24550318-24551148 | chr18: 70208975-70211790 |
| chr10: 102416497-102416716 | chr14: 24835811-24836220 | chr18: 72123607-72124717 |
| [symbol] 698-102431119 | chr14: 24837873-24838324 | chr18: 74514041-74514292 |
| chr10: 102440601-102441011 | chr14: 24898780-24899288 | chr18: 74843361-74845426 |
| chr10: 102469807-102470341 | chr14: 25518425-25519612 | chr18: 7566558-7568830 |
| chr10: 102501701-102502232 | chr14: 27066314-27066578 | chr18: 77724353-77724836 |
| chr10: 102586126-102588109 | chr14: 32670418-32671092 | chr18: 811650-812709 |
| chr10: 102590123-102590402 | chr14: 34419509-34420438 | chr18: 8367274-8367599 |
| chr10: 102778605-102778922 | chr14: 37641233-37641879 | chr18: 9333897-9334244 |
| chr10: 102792043-102792266 | chr14: 38052646-38054166 | chr19: 10077032-10077444 |
| chr10: 102807775-102808271 | chr14: 38057139-38057798 | chr19: 10120751-10121231 |
| chr10: 102809888-102810282 | chr14: 38060842-38062119 | chr19: 10526983-10527755 |
| chr10: 102882978-102883551 | chr14: 38063664-38065665 | chr19: 10529628-10532004 |
| chr10: 103326283-103326712 | chr14: 38067447-38069207 | chr19: 1068439-1068764 |
| chr10: 103538816-103539988 | chr14: 45431629-45432115 | chr19: 10697787-10698099 |
| chr10: 103985853-103986246 | chr14: 52780688-52781969 | chr19: 1074727-1075071 |
| chr10: 104000255-104001741 | chr14: 54413305-54413764 | chr19: 11353961-11354362 |
| chr10: 105211951-105212306 | chr14: 55595698-55596692 | chr19: 11491560-11492740 |
| chr10: 106028543-106029047 | chr14: 58862542-58863209 | chr19: 11529214-11529588 |
| chr10: 110225928-110226465 | chr14: 60097209-60097553 | chr19: 11529723-11529966 |
| chr10: 11059443-11060524 | chr14: 60794577-60794867 | chr19: 11531279-11531590 |
| chr10: 11207179-11207980 | chr14: 61114103-61116552 | chr19: 11533199-11533619 |
| chr10: 113943284-113943657 | chr14: 61746805-61748141 | chr19: 11689418-11689768 |
| chr10: 115860680-115861093 | chr14: 61787881-61789467 | chr19: 11909403-11909841 |
| chr10: 116391467-116391827 | chr14: 62583680-62584279 | chr19: 11959578-11960064 |
| chr10: 116527525-116528474 | chr14: 67878535-67879167 | chr19: 11998805-11999131 |
| chr10: 116852262-116854094 | chr14: 70041214-70041662 | chr19: 12076030-12076366 |
| chr10: 118976050-118976706 | chr14: 73704055-73705106 | chr19: 12098572-12098950 |
| chr10: 119293918-119297675 | chr14: 73706300-73706677 | chr19: 12146031-12146649 |
| chr10: 122708511-122708899 | chr14: 73957751-73958889 | chr19: 12163327-12163560 |
| chr10: 122739053-122739391 | chr14: 74036128-74036662 | chr19: 12175461-12176057 |
| chr10: 124638744-124639793 | chr14: 74058401-74059116 | chr19: 12266999-12267686 |
| chr10: 125851152-125853233 | chr14: 74100344-74101230 | chr19: 12305553-12306304 |
| chr10: 126135810-126138896 | chr14: 75389713-75390133 | chr19: 12444033-12444548 |
| chr10: 133879006-133879255 | chr14: 75894309-75895469 | chr19: 12476050-12476372 |
| chr10: 134973511-134974311 | chr14: 76819146-76819379 | chr19: 12943347-12943661 |
| chr10: 135075002-135075452 | chr14: 88789601-88790493 | chr19: 12978360-12978785 |
| chr10: 135089970-135090491 | chr14: 89493136-89494382 | chr19: 13207376-13207621 |
| chr10: 13933483-13934184 | chr14: 90167783-90168352 | chr19: 13208344-13210525 |
| chr10: 17270431-17272617 | chr14: 91883362-91883566 | chr19: 1324970-1325349 |
| chr10: 17495408-17496721 | chr14: 92302202-92303078 | chr19: 13266390-13267030 |
| chr10: 18429126-18430234 | chr14: 92413582-92414324 | chr19: 1401093-1401784 |
| chr10: 20104452-20106105 | chr14: 96505312-96506402 | chr19: 14168143-14168512 |
| [symbol] 44-20106946 | chr14: 96890431-96890633 | chr19: 14542769-14543102 |
| chr10: 21462129-21463808 | chr14: 97685060-97685946 | chr19: 1455227-1455544 |
| chr10: 22518027-22518334 | chr14: 99711840-99713431 | chr19: 1456078-1456347 |
| chr10: 25241111-25241900 | chr14: 99739802-99740920 | chr19: 1456886-1457312 |
| chr10: 25463757-25465639 | chr15: 100880958-100882438 | chr19: 1503512-1503791 |
| chr10: 26222922-26224022 | chr15: 100890243-100890494 | chr19: 15090017-15090873 |
| chr10: 26500608-26501126 | chr15: 101389733-101390260 | chr19: 15288315-15288911 |
| chr10: 26680635-26680999 | chr15: 101548676-101548898 | chr19: 15334123-15334822 |
| chr10: 28030183-28035211 | chr15: 23207950-23208796 | chr19: 15619224-15619613 |
| chr10: 29698363-29699044 | chr15: 24123232-24123432 | chr19: 15662114-15662358 |
| chr10: 31422710-31423316 | chr15: 28982670-28983497 | chr19: 15695299-15695535 |
| chr10: 35103125-35105243 | chr15: 30113706-30113940 | chr19: 16186790-16188275 |
| chr10: 43571940-43573434 | chr15: 31733082-31733489 | chr19: 1675922-1676137 |
| chr10: 44185040-44186132 | chr15: 32828333-32829181 | chr19: 17007102-17008857 |
| chr10: 45470023-45470291 | chr15: 34728788-34729495 | chr19: 1725416-1726154 |
| chr10: 45914375-45914883 | chr15: 37179517-37179782 | chr19: 17346166-17346886 |
| chr10: 49812853-49813352 | chr15: 37393667-37394248 | chr19: 17392384-17393775 |
| chr10: 51572199-51572620 | chr15: 40728263-40728466 | chr19: 17403040-17403570 |
| chr10: 52177725-52178182 | chr15: 41851724-41851970 | chr19: 17487896-17488125 |
| chr10: 57390344-57391215 | chr15: 41913334-41914360 | chr19: 17501956-17502250 |
| chr10: 60272468-60273456 | chr15: 41952536-41953222 | chr19: 17580198-17580598 |
| chr10: 60935828-60937049 | chr15: 45427297-45428206 | chr19: 1762373-1762848 |
| chr10: 62492786-62493376 | chr15: 45722562-45722939 | chr19: 1766145-1766364 |
| chr10: 62703308-62704290 | chr15: 48009419-48011044 | chr19: 17716645-17717531 |
| chr10: 64133790-64134420 | chr15: 48470008-48470628 | chr19: 1776028-1776730 |
| chr10: 71077947-71079377 | chr15: 51633725-51634318 | chr19: 17791068-17791301 |
| chr10: 71812319-71813693 | chr15: 52587354-52588172 | chr19: 17905580-17906317 |
| chr10: 71892093-71892802 | chr15: 60690709-60690930 | chr19: 18059850-18060148 |
| chr10: 72043447-72043886 | chr15: 61519622-61520031 | chr19: 18209912-18210268 |
| chr10: 72218131-72218484 | chr15: 61520424-61521716 | chr19: 18319146-18319363 |
| chr10: 72647739-72648317 | chr15: 62456016-62457648 | chr19: 18499098-18499697 |
| chr10: 72977124-72977774 | chr15: 64443588-64444821 | chr19: 18539782-18540341 |
| chr10: 734708-735606 | chr15: 65066917-65068352 | chr19: 1862257-1862600 |
| chr10: 74020774-74021023 | chr15: 65127965-65128185 | chr19: 18722359-18724001 |
| chr10: 74079337-74079861 | chr15: 65669311-65670617 | chr19: 18811562-18811791 |
| chr10: 75670697-75671379 | chr15: 65714743-65715571 | chr19: 19280999-19281560 |
| chr10: 77191062-77191571 | chr15: 66274584-66274838 | chr19: 19335772-19336151 |
| chr10: 79396096-79398495 | chr15: 66544433-66546134 | chr19: 19729128-19729814 |
| chr10: 8091375-8098329 | chr15: 68126023-68126569 | chr19: 19738573-19739821 |
| chr10: 81892223-81892640 | chr15: 68723368-68724691 | chr19: 19843483-19843943 |
| chr10: 85954200-85955303 | chr15: 69366322-69366796 | chr19: 19971578-19971863 |
| [symbol] 00-86300953 | chr15: 71145996-71146820 | chr19: 20011911-20012238 |
| chr10: 88730555-88731632 | chr15: 72476677-72476969 | chr19: 20149856-20150175 |
| chr10: 90342529-90343221 | chr15: 72564443-72565165 | chr19: 20162773-20163107 |
| chr10: 93647053-93647393 | chr15: 72611947-72612802 | chr19: 2041824-2042593 |
| chr10: 94351357-94351615 | chr15: 74044562-74045885 | chr19: 21949904-21950217 |
| chr10: 94459255-94459591 | chr15: 74425092-74428821 | chr19: 22018595-22018827 |
| chr10: 94831513-94832312 | chr15: 74537810-74538114 | chr19: 22034395-22034943 |
| chr10: 95326230-95327108 | chr15: 75470912-75471212 | chr19: 22193331-22193583 |
| chr10: 95360390-95361387 | chr15: 75628251-75628677 | chr19: 2273694-2274047 |
| chr10: 95753348-95754201 | chr15: 78633323-78634089 | chr19: 2302467-2302999 |
| chr10: 97802872-97804262 | chr15: 79102716-79104628 | chr19: 23253797-23254382 |
| chr10: 98479726-98480487 | chr15: 83348840-83349865 | chr19: 23386650-23387069 |
| chr10: 99473085-99473291 | chr15: 83620951-83621727 | chr19: 23456575-23456868 |
| chr11: 100998031-100999774 | chr15: 83775862-83776922 | chr19: 2478866-2479244 |
| chr11: 101918074-101918493 | chr15: 83875649-83877079 | chr19: 2613933-2614133 |
| chr11: 102188180-102188440 | chr15: 84047823-84048105 | chr19: 267086-267660 |
| chr11: 106888324-106890203 | chr15: 84115763-84116921 | chr19: 28284529-28285129 |
| chr11: 109963241-109964677 | chr15: 84322323-84323771 | chr19: 295115-295354 |
| chr11: 112832525-112834490 | chr15: 84976071-84977044 | chr19: 30215233-30215594 |
| chr11: 113185333-113185663 | chr15: 89438177-89438850 | chr19: 30363203-30363527 |
| chr11: 113258444-113258821 | chr15: 89942593-89943853 | chr19: 31839636-31843049 |
| chr11: 113345069-113346328 | chr15: 89959743-89959990 | chr19: 31843881-31844183 |
| chr11: 113929634-113932190 | chr15: 90039465-90039984 | chr19: 31847947-31848209 |
| chr11: 113953621-113953839 | chr15: 91427541-91427905 | chr19: 3275609-3275970 |
| chr11: 116371183-116371606 | chr15: 93198375-93199181 | chr19: 33685147-33686085 |
| chr11: 116706482-116706910 | chr15: 93615623-93617185 | chr19: 34112280-34114353 |
| chr11: 119227097-119227758 | chr15: 96883942-96884533 | chr19: 3434930-3435417 |
| chr11: 119455154-119456102 | chr15: 96888959-96889417 | chr19: 34396472-34398079 |
| chr11: 119612092-119612476 | chr15: 99091356-99091812 | chr19: 34850072-34850587 |
| chr11: 120039602-120040210 | chr15: 99557979-99559170 | chr19: 35531781-35531991 |
| chr11: 120110498-120110719 | chr16: 10479687-10480254 | chr19: 36164078-36164286 |
| chr11: 120856726-120857174 | chr16: 1098523-1098875 | chr19: 36351441-36352167 |
| chr11: 121322539-121323302 | chr16: 11327009-11327234 | chr19: 3671903-3672121 |
| chr11: 122854931-122855582 | chr16: 1202415-1204624 | chr19: 3687787-3688262 |
| chr11: 123301050-123302149 | chr16: 12897366-12897889 | chr19: 36909282-36909854 |
| chr11: 124709223-124709957 | chr16: 1392545-1392753 | chr19: 37063893-37064749 |
| chr11: 124713006-124713395 | chr16: 1429051-1430104 | chr19: 37095681-37096589 |
| chr11: 124932731-124933657 | chr16: 1458144-1458435 | chr19: 37157633-37158119 |
| chr11: 126225356-126226073 | chr16: 19421817-19422498 | chr19: 3721465-3721788 |
| chr11: 126286452-126286870 | chr16: 203754-204866 | chr19: 37263382-37264083 |
| chr11: 12695415-12696981 | chr16: 2054012-2054288 | chr19: 37328897-37329518 |
| [symbol] 713-128392611 | chr16: 2069719-2070865 | chr19: 37340919-37341262 |
| chr11: 129243999-129244567 | chr16: 2077043-2077470 | chr19: 37341732-37342000 |
| chr11: 129245109-129246395 | chr16: 21294981-21295414 | chr19: 37568953-37569549 |
| chr11: 129685738-129686211 | chr16: 21831483-21831944 | chr19: 37803517-37803822 |
| chr11: 130029659-130030262 | chr16: 2245867-2247100 | chr19: 37957727-37958390 |
| chr11: 130297402-130298517 | chr16: 25078048-25078532 | chr19: 37959853-37960615 |
| chr11: 133825338-133827457 | chr16: 2835064-2835958 | chr19: 38145827-38147219 |
| chr11: 134201785-134202407 | chr16: 28634441-28635031 | chr19: 38182794-38183327 |
| chr11: 13689589-13690724 | chr16: 28752083-28752332 | chr19: 38210108-38210692 |
| chr11: 15094958-15095872 | chr16: 29624388-29625391 | chr19: 3821975-3822278 |
| chr11: 1592499-1592810 | chr16: 29887789-29888316 | chr19: 38754847-38755699 |
| chr11: 16632509-16632725 | chr16: 30428871-30429799 | chr19: 38852312-38853485 |
| chr11: 17373020-17373665 | chr16: 3078142-3078553 | chr19: 39359917-39361160 |
| chr11: 17565767-17566052 | chr16: 3079537-3079954 | chr19: 39440405-39441011 |
| chr11: 18067717-18067928 | chr16: 30913356-30913942 | chr19: 39574701-39575137 |
| chr11: 18415923-18416680 | chr16: 31008833-31009036 | chr19: 39687598-39687964 |
| chr11: 18727321-18727727 | chr16: 31117102-31117435 | chr19: 39810993-39811293 |
| chr11: 19798538-19798949 | chr16: 31213567-31214287 | chr19: 39894559-39894768 |
| chr11: 20385162-20385673 | chr16: 31342453-31343140 | chr19: 39993358-39993765 |
| chr11: 2165136-2165672 | chr16: 31483277-31483646 | chr19: 39997591-39998291 |
| chr11: 22362863-22363377 | chr16: 31580071-31580317 | chr19: 40323908-40325005 |
| chr11: 22850766-22851367 | chr16: 31580560-31581023 | chr19: 40732076-40732665 |
| chr11: 2290105-2292932 | chr16: 3199653-3199937 | chr19: 41025315-41026106 |
| chr11: 2465172-2465648 | chr16: 3225355-3225594 | chr19: 41055125-41055331 |
| chr11: 2466248-2466818 | chr16: 32822902-32823585 | chr19: 41060059-41060565 |
| chr11: 27740645-27741236 | chr16: 3355021-3356012 | chr19: 41106059-41106415 |
| chr11: 30040073-30040294 | chr16: 4166656-4167012 | chr19: 41109614-41110075 |
| chr11: 30606005-30608128 | chr16: 4357553-4357955 | chr19: 41111084-41111767 |
| chr11: 3181575-3182120 | chr16: 4363936-4364151 | chr19: 41115446-41115767 |
| chr11: 32354761-32355484 | chr16: 4377509-4378162 | chr19: 41119032-41120394 |
| chr11: 32448262-32449744 | chr16: 4421643-4422240 | chr19: 41633777-41634047 |
| chr11: 35547140-35547396 | chr16: 4587837-4588890 | chr19: 41698744-41699325 |
| chr11: 416420-417650 | chr16: 46823933-46824263 | chr19: 42419339-42419564 |
| chr11: 43596991-43597336 | chr16: 47176788-47178446 | chr19: 42501262-42501486 |
| chr11: 43902255-43902528 | chr16: 48399731-48400040 | chr19: 42720827-42722132 |
| chr11: 45307442-45307975 | chr16: 50581476-50582735 | chr19: 42901017-42901375 |
| chr11: 45686161-45687495 | chr16: 52579637-52580845 | chr19: 42905724-42906350 |
| chr11: 45921388-45922167 | chr16: 52581294-52581610 | chr19: 42927286-42928598 |
| chr11: 46316876-46317485 | chr16: 54316904-54317149 | chr19: 43979360-43979684 |
| chr11: 46389075-46389378 | chr16: 54962423-54967805 | chr19: 44324437-44325009 |
| chr11: 47736740-47737106 | chr16: 55357522-55359040 | chr19: 44405818-44406053 |
| [symbol] 18-49230040 | chr16: 56224732-56224980 | chr19: 44763979-44764312 |
| chr11: 504190-504659 | chr16: 56225256-56226351 | chr19: 44952417-44952809 |
| chr11: 57117316-57117575 | chr16: 56677121-56677598 | chr19: 45257704-45257918 |
| chr11: 57243682-57244463 | chr16: 56701864-56702208 | chr19: 45281134-45281355 |
| chr11: 57249947-57250848 | chr16: 56703388-56703648 | chr19: 4535070-4535339 |
| chr11: 58940831-58941052 | chr16: 57835956-57836989 | chr19: 4558364-4558579 |
| chr11: 61061752-61063063 | chr16: 58058713-58058928 | chr19: 45655294-45657246 |
| chr11: 61159837-61160285 | chr16: 58535041-58535596 | chr19: 45843871-45844418 |
| chr11: 61283865-61284147 | chr16: 66638255-66639561 | chr19: 45888769-45889465 |
| chr11: 61322733-61323285 | chr16: 66982435-66982945 | chr19: 45947711-45948047 |
| chr11: 61335113-61335340 | chr16: 67034471-67035340 | chr19: 46032305-46032855 |
| chr11: 61355039-61355363 | chr16: 67188614-67189652 | chr19: 4607080-4607350 |
| chr11: 61519742-61519972 | chr16: 67193006-67193219 | chr19: 46144873-46145631 |
| chr11: 61722745-61723483 | chr16: 67196793-67199988 | chr19: 46282545-46283127 |
| chr11: 62476771-62477481 | chr16: 67204209-67204637 | chr19: 46379884-46380207 |
| chr11: 62690975-62691488 | chr16: 67211934-67212509 | chr19: 46456210-46456503 |
| chr11: 62693374-62694772 | chr16: 67312897-67313731 | chr19: 46518284-46520080 |
| chr11: 63685490-63685968 | chr16: 67564059-67564332 | chr19: 46974558-46975073 |
| chr11: 63775347-63775613 | chr16: 67700010-67701354 | chr19: 46996328-46998437 |
| chr11: 63803376-63804365 | chr16: 68002838-68003039 | chr19: 47137781-47138070 |
| chr11: 64038965-64039306 | chr16: 68273546-68274084 | chr19: 47614409-47614661 |
| chr11: 64409878-64411253 | chr16: 68678687-68680149 | chr19: 47910108-47910563 |
| chr11: 64993281-64993484 | chr16: 69760294-69760506 | chr19: 47922252-47922777 |
| chr19: 48216487-48216837 | chr22: 23522551-23524465 | chr6: 10410110-10410690 |
| chr19: 48833395-48833720 | chr22: 23908323-23909178 | chr6: 10414217-10414498 |
| chr19: 48837142-48837549 | chr22: 24110030-24110995 | chr6: 10415019-10415318 |
| chr19: 48901805-48902123 | chr22: 24551814-24552696 | chr6: 10417385-10417842 |
| chr19: 49061546-49061769 | chr22: 26565200-26565986 | chr6: 10419400-10420323 |
| chr19: 49199965-49200184 | chr22: 27152698-27153133 | chr6: 10421415-10421727 |
| chr19: 49242019-49242962 | chr22: 29426511-29426843 | chr6: 10422322-10422648 |
| chr19: 49255779-49256495 | chr22: 29467292-29467817 | chr6: 10426201-10426638 |
| chr19: 49340489-49340774 | chr22: 31218227-31218893 | chr6: 105627407-105627830 |
| chr19: 49522774-49523093 | chr22: 32026298-32026874 | chr6: 106958233-106958718 |
| chr19: 49575130-49576076 | chr22: 33453893-33454505 | chr6: 106959765-106960985 |
| chr19: 49646093-49646308 | chr22: 37655606-37655839 | chr6: 108436110-108436522 |
| chr19: 49655103-49655395 | chr22: 37730552-37731415 | chr6: 108455061-108455273 |
| chr19: 49935752-49936275 | chr22: 38073038-38073412 | chr6: 112575092-112575483 |
| chr19: 49939611-49940045 | chr22: 38453188-38453503 | chr6: 117584585-117584816 |
| chr19: 50016532-50016851 | chr22: 38808785-38809017 | chr6: 118241348-118241569 |
| chr19: 50030982-50031300 | chr22: 39745984-39746634 | chr6: 124124178-124125309 |
| chr19: 50037079-50037670 | chr22: 39853324-39854065 | chr6: 125283125-125284389 |
| [symbol] 10-50096912 | chr22: 40057942-40058844 | chr6: 125420744-125421141 |
| chr19: 50183923-50184172 | chr22: 40390485-40391490 | chr6: 127836731-127837706 |
| chr19: 50312012-50312821 | chr22: 42062791-42063002 | chr6: 1311069-1314220 |
| chr19: 50651174-50651535 | chr22: 42470036-42470669 | chr6: 132271357-132271658 |
| chr19: 50706483-50707196 | chr22: 43505952-43506167 | chr6: 134159127-134159349 |
| chr19: 50708602-50708855 | chr22: 45015934-45016488 | chr6: 134213960-134214351 |
| chr19: 50831455-50832070 | chr22: 45403037-45406372 | chr6: 13873766-13874262 |
| chr19: 50833814-50834128 | chr22: 46262066-46263747 | chr6: 13925100-13925510 |
| chr19: 50836391-50837447 | chr22: 46366727-46368726 | chr6: 146136326-146136564 |
| chr19: 51069360-51069600 | chr22: 50328694-50329178 | chr6: 150246753-150247257 |
| chr19: 51161924-51162514 | chr22: 50453057-50454137 | chr6: 150284683-150286515 |
| chr19: 51198809-51199347 | chr22: 50628950-50629249 | chr6: 150463772-150465002 |
| chr19: 51227662-51228883 | chr22: 50982571-50982994 | chr6: 151561284-151562550 |
| chr19: 51321268-51321781 | chr3: 101497831-101498648 | chr6: 151646669-151646958 |
| chr19: 51416003-51416249 | chr3: 101568108-101569046 | chr6: 152128823-152129771 |
| chr19: 51522005-51522803 | chr3: 10206450-10207024 | chr6: 152623280-152623480 |
| chr19: 51568155-51568412 | chr3: 112930438-112931506 | chr6: 152957811-152958472 |
| chr19: 51842129-51842353 | chr3: 113251700-113252290 | chr6: 155316300-155317197 |
| chr19: 52206245-52206726 | chr3: 117716101-117716564 | chr6: 158957221-158958677 |
| chr19: 52207133-52207731 | chr3: 119041383-119042255 | chr6: 159290674-159291195 |
| chr19: 52222424-52223208 | chr3: 119421856-119422334 | chr6: 1604607-1615866 |
| chr19: 52452317-52452543 | chr3: 120003884-120004426 | chr6: 166267510-166268461 |
| chr19: 52531324-52531724 | chr3: 120626881-120627579 | chr6: 166401527-166402659 |
| chr19: 52800400-52800871 | chr3: 122640714-122641355 | chr6: 17102310-17102539 |
| chr19: 52839445-52839937 | chr3: 12328995-12329227 | chr6: 19837505-19839314 |
| chr19: 52900956-52901158 | chr3: 124303530-124304092 | chr6: 21594614-21596437 |
| chr19: 5293011-5294211 | chr3: 124931162-124931747 | chr6: 21664508-21665178 |
| chr19: 53030825-53031359 | chr3: 126242784-126243358 | chr6: 21665716-21666031 |
| chr19: 53073309-53074039 | chr3: 127391012-127392434 | chr6: 21666656-21666862 |
| chr19: 53104385-53105149 | chr3: 128145258-128145656 | chr6: 24360030-24360411 |
| chr19: 53141176-53141813 | chr3: 128205496-128212274 | chr6: 25652381-25652709 |
| chr19: 53193140-53193945 | chr3: 128336407-128337113 | chr6: 26020672-26021125 |
| chr19: 5339641-5341061 | chr3: 128564772-128565007 | chr6: 26021958-26022193 |
| chr19: 53400461-53401205 | chr3: 129323916-129325332 | chr6: 26044204-26044469 |
| chr19: 53426079-53426389 | chr3: 129345668-129346521 | chr6: 26045645-26046033 |
| chr19: 53496733-53497028 | chr3: 129721369-129722131 | chr6: 26172034-26172398 |
| chr19: 53606060-53606784 | chr3: 13008649-13009210 | chr6: 26184037-26184336 |
| chr19: 53635626-53636230 | chr3: 131753881-131754139 | chr6: 26197071-26197537 |
| chr19: 53661431-53661848 | chr3: 133464950-133465420 | chr6: 26199198-26199466 |
| chr19: 53662190-53662500 | chr3: 134082861-134083201 | chr6: 26204765-26205213 |
| chr19: 53696030-53696650 | chr3: 134125488-134125871 | chr6: 26217212-26217521 |
| [symbol] 69-54024560 | chr3: 136751342-136751683 | chr6: 26240698-26240951 |
| chr19: 54024646-54024923 | chr3: 138153270-138154621 | chr6: 26250437-26250827 |
| chr19: 54369388-54369809 | chr3: 140769887-140771080 | chr6: 26251899-26252233 |
| chr19: 54382665-54382951 | chr3: 145878431-145879287 | chr6: 26271346-26271588 |
| chr19: 54409967-54410200 | chr3: 145968335-145969008 | chr6: 26272471-26272696 |
| chr19: 54599161-54599456 | chr3: 150802997-150805168 | chr6: 26273291-26273557 |
| chr19: 54665922-54666377 | chr3: 152552603-152553712 | chr6: 26987599-26988074 |
| chr19: 54982389-54982897 | chr3: 156533840-156535131 | chr6: 27100175-27101124 |
| chr19: 5536532-5536789 | chr3: 157155268-157155826 | chr6: 27107139-27107394 |
| chr19: 55591434-55591644 | chr3: 158288801-158289271 | chr6: 27598688-27599146 |
| chr19: 55591906-55593980 | chr3: 159756633-159756997 | chr6: 27777856-27778251 |
| chr19: 55667533-55668465 | chr3: 160822495-160823260 | chr6: 27782248-27782485 |
| chr19: 55672024-55673212 | chr3: 168864031-168864449 | chr6: 27791781-27792217 |
| chr19: 5567519-5567939 | chr3: 169482339-169483052 | chr6: 27798991-27799427 |
| chr19: 55865017-55865434 | chr3: 169487146-169487522 | chr6: 27835191-27835461 |
| chr19: 55865825-55866324 | chr3: 173113271-173113681 | chr6: 28367125-28367651 |
| chr19: 55953372-55954641 | chr3: 179168736-179169593 | chr6: 2841811-2842273 |
| chr19: 56159258-56159937 | chr3: 179754521-179755245 | chr6: 2875602-2875811 |
| chr19: 56728589-56729375 | chr3: 181413015-181414022 | chr6: 2971087-2971962 |
| chr19: 56879418-56879995 | chr3: 181430142-181431076 | chr6: 3231596-3232289 |
| chr19: 56904637-56905355 | chr3: 181437185-181437478 | chr6: 32632159-32633027 |
| chr19: 56915357-56915856 | chr3: 182896921-182897381 | chr6: 33539023-33539339 |
| chr19: 57049676-57050646 | chr3: 182971430-182972635 | chr6: 36098040-36098621 |
| chr19: 57078366-57079301 | chr3: 183542497-183543804 | chr6: 39196818-39197517 |
| chr19: 57106576-57106896 | chr3: 184319377-184319822 | chr6: 40554596-40555928 |
| chr19: 57149424-57149631 | chr3: 184320006-184320218 | chr6: 40995803-40996241 |
| chr19: 57276615-57276942 | chr3: 184321784-184322277 | chr6: 41339237-41340027 |
| chr19: 57702697-57703425 | chr3: 18485113-18487056 | chr6: 41410590-41411140 |
| chr19: 57831600-57832250 | chr3: 184971642-184972002 | chr6: 41438063-41438594 |
| chr19: 57862442-57863236 | chr3: 185000558-185000896 | chr6: 42109905-42110361 |
| chr19: 57874764-57875110 | chr3: 186490603-186490813 | chr6: 4282005-4282310 |
| chr19: 57999026-57999613 | chr3: 186648175-186649103 | chr6: 42928219-42928810 |
| chr19: 58011125-58011743 | chr3: 187455331-187455843 | chr6: 43021126-43021694 |
| chr19: 5802235-5802569 | chr3: 187456251-187456580 | chr6: 43044045-43045057 |
| chr19: 58038573-58039208 | chr3: 187457732-187457948 | chr6: 43142014-43142217 |
| chr19: 58070554-58071273 | chr3: 188665276-188665552 | chr6: 43237261-43237643 |
| chr19: 58111230-58111770 | chr3: 193720954-193721291 | chr6: 43970074-43970922 |
| chr19: 58125531-58125902 | chr3: 193776092-193776308 | chr6: 44119527-44119781 |
| chr19: 58175624-58176104 | chr3: 193858771-193859695 | chr6: 44187187-44187400 |
| chr19: 58220190-58220517 | chr3: 194117602-194118988 | chr6: 45387347-45388164 |
| chr19: 58280927-58281486 | chr3: 194207386-194208785 | chr6: 4775132-4777550 |
| [symbol] 37-58446800 | chr3: 196255496-196256013 | chr6: 49518316-49519186 |
| chr19: 58458687-58459219 | chr3: 197281606-197283128 | chr6: 52441270-52441793 |
| chr19: 58609339-58609988 | chr3: 197391890-197392481 | chr6: 53212618-53214043 |
| chr19: 58661738-58662287 | chr3: 23244051-23245071 | chr6: 54711154-54712033 |
| chr19: 58666024-58666833 | chr3: 24535845-24537436 | chr6: 56407465-56407926 |
| chr19: 58739944-58740554 | chr3: 24870675-24871369 | chr6: 56818874-56820308 |
| chr19: 58858454-58859223 | chr3: 27410613-27411066 | chr6: 69345217-69345483 |
| chr19: 58951215-58952250 | chr3: 27756213-27756422 | chr6: 70576975-70577572 |
| chr19: 59025309-59025705 | chr3: 27756661-27756941 | chr6: 710835-711297 |
| chr19: 6199293-6199551 | chr3: 32021970-32023364 | chr6: 7141037-7141576 |
| chr19: 6463992-6464780 | chr3: 32443153-32443455 | chr6: 72129509-72130756 |
| chr19: 6530827-6531552 | chr3: 38035702-38036000 | chr6: 73972820-73973027 |
| chr19: 6740670-6741203 | chr3: 38040290-38040535 | chr6: 74019429-74020053 |
| chr19: 675672-675881 | chr3: 38080629-38081187 | chr6: 74024403-74025020 |
| chr19: 6767946-6768160 | chr3: 38179858-38180689 | chr6: 74161087-74162090 |
| chr19: 681346-681576 | chr3: 40428652-40429015 | chr6: 74233269-74233546 |
| chr19: 7953281-7953708 | chr3: 40657160-40657395 | chr6: 75794881-75795088 |
| chr19: 8273531-8273920 | chr3: 44690127-44690587 | chr6: 75914706-75916387 |
| chr19: 8274242-8275045 | chr3: 44726929-44727237 | chr6: 83073417-83075319 |
| chr19: 9473590-9474001 | chr3: 44754100-44754399 | chr6: 87647254-87647707 |
| chr19: 9896564-9896994 | chr3: 44770937-44771137 | chr6: 94126273-94129677 |
| chr2: 100937780-100939059 | chr3: 45187027-45187946 | chr6: 96463870-96464136 |
| chr2: 101033607-101034296 | chr3: 46734732-46735717 | chr6: 99279318-99283842 |
| chr2: 10260280-10260931 | chr3: 46742765-46743086 | chr7: 100091181-100091598 |
| chr2: 102758807-102759577 | chr3: 46887629-46887919 | chr7: 100167137-100168144 |
| chr2: 105488369-105489991 | chr3: 46940055-46940394 | chr7: 100201615-100203652 |
| chr2: 105760110-105761018 | chr3: 50275394-50275645 | chr7: 100224271-100224558 |
| chr2: 106886119-106886738 | chr3: 50377804-50378540 | chr7: 100230650-100231320 |
| chr2: 107502354-107504216 | chr3: 50402104-50402942 | chr7: 100434983-100435193 |
| chr2: 10861207-10862382 | chr3: 50604906-50605359 | chr7: 100845051-100845726 |
| chr2: 110370907-110373301 | chr3: 51989764-51990639 | chr7: 102157824-102158510 |
| chr2: 110518072-110518913 | chr3: 52279602-52280140 | chr7: 102329951-102330654 |
| chr2: 112656187-112656918 | chr3: 53190510-53190764 | chr7: 104624338-104624848 |
| chr2: 119981080-119981818 | chr3: 54155240-54157025 | chr7: 106684814-106685765 |
| chr2: 121101047-121101432 | chr3: 55515153-55515613 | chr7: 108095402-108097206 |
| chr2: 121101801-121104534 | chr3: 55517659-55517939 | chr7: 114562171-114563193 |
| chr2: 121104713-121104935 | chr3: 55519090-55519623 | chr7: 115850371-115851028 |
| chr2: 121199724-121199993 | chr3: 55520227-55522344 | chr7: 116139775-116140352 |
| chr2: 121200504-121200788 | chr3: 55522561-55522836 | chr7: 121513047-121513911 |
| chr2: 121499229-121499578 | chr3: 56501869-56502345 | chr7: 12610166-12610834 |
| chr2: 121624828-121625209 | chr3: 57198244-57199378 | chr7: 126891301-126894205 |
| [symbol] 01-127644104 | chr3: 57994922-57995218 | chr7: 126987644-126988233 |
| chr2: 127729673-127729905 | chr3: 59035407-59035944 | chr7: 127880751-127881375 |
| chr2: 128173476-128173793 | chr3: 62304515-62304780 | chr7: 128509207-128510201 |
| chr2: 128180295-128181381 | chr3: 62859618-62861190 | chr7: 128530630-128531356 |
| chr2: 130691703-130692102 | chr3: 64430586-64431192 | chr7: 128809090-128809393 |
| chr2: 131129719-131130511 | chr3: 65534685-65535344 | chr7: 128828334-128829366 |
| chr2: 131513364-131514183 | chr3: 69129059-69129515 | chr7: 130126018-130126801 |
| chr2: 131720820-131721867 | chr3: 69434927-69435583 | chr7: 130417913-130419378 |
| chr2: 131792238-131793189 | chr3: 69591012-69592050 | chr7: 132260475-132262527 |
| chr2: 132121264-132121762 | chr3: 72788073-72788416 | chr7: 1329382-1329673 |
| chr2: 132285409-132285992 | chr3: 73673231-73674375 | chr7: 133812006-133812233 |
| chr2: 133173533-133174369 | chr3: 74663507-74664121 | chr7: 138666359-138666972 |
| chr2: 133426653-133428870 | chr3: 75834255-75834761 | chr7: 138720045-138721019 |
| chr2: 134023948-134024467 | chr3: 77088499-77089198 | chr7: 139167481-139168712 |
| chr2: 134785626-134785845 | chr3: 85008285-85008837 | chr7: 14029379-14029593 |
| chr2: 145273457-145275378 | chr3: 96532024-96533625 | chr7: 1408517-1408878 |
| chr2: 145281737-145282269 | chr3: 98451287-98452225 | chr7: 148036495-148036848 |
| chr2: 14772378-14775809 | chr3: 98620132-98620910 | chr7: 148982080-148982675 |
| chr2: 154334450-154335458 | chr3: 9904332-9904811 | chr7: 149119399-149120307 |
| chr2: 155554006-155556069 | chr3: 9956823-9957664 | chr7: 149495-150038 |
| chr2: 160654230-160654631 | chr3: 99594970-99595215 | chr7: 150019951-150020752 |
| chr2: 160918675-160919177 | chr3: 9993731-9994136 | chr7: 150655109-150655643 |
| chr2: 161263872-161264460 | chr4: 1005931-1006282 | chr7: 150675207-150675866 |
| chr2: 162930234-162930879 | chr4: 102711830-102712199 | chr7: 150676179-150676585 |
| chr2: 164592918-164593511 | chr4: 103997209-103998007 | chr7: 151078463-151079536 |
| chr2: 166649910-166650966 | chr4: 10462833-10463689 | chr7: 151136934-151137273 |
| chr2: 168149034-168150797 | chr4: 1049653-1049923 | chr7: 154861798-154862074 |
| chr2: 171569878-171573904 | chr4: 109683730-109684362 | chr7: 155579869-155580392 |
| chr2: 172944989-172945384 | chr4: 111532648-111533391 | chr7: 156810573-156814593 |
| chr2: 172945912-172946212 | chr4: 113435899-113438002 | chr7: 158785262-158785771 |
| chr2: 172947844-172948200 | chr4: 113445023-113445350 | chr7: 16460779-16461265 |
| chr2: 172949243-172950126 | chr4: 1161128-1161682 | chr7: 1703862-1710517 |
| chr2: 172951290-172952159 | chr4: 118006539-118006859 | chr7: 191876-193799 |
| chr2: 172952810-172953160 | chr4: 119273306-119274458 | chr7: 20817456-20818227 |
| chr2: 172959268-172962054 | chr4: 123747741-123748980 | chr7: 20823308-20825662 |
| chr2: 172971046-172971387 | chr4: 124333-124841 | chr7: 21582444-21583313 |
| chr2: 173600282-173600970 | chr4: 124426781-124427157 | chr7: 21985072-21985842 |
| chr2: 1746834-1748971 | chr4: 126235896-126238930 | chr7: 22893795-22894577 |
| chr2: 174877566-174877778 | chr4: 13529255-13529865 | chr7: 23513535-23514412 |
| chr2: 175193399-175193764 | chr4: 13548571-13549956 | chr7: 24323559-24325080 |
| chr2: 175199464-175202639 | chr4: 1396292-1401730 | chr7: 25891957-25892615 |
| [symbol] 44-175207553 | chr4: 141294667-141295274 | chr7: 25898238-25898771 |
| chr2: 176989338-176989587 | chr4: 142053328-142054601 | chr7: 26191795-26192757 |
| chr2: 176993480-176995557 | chr4: 154143933-154144463 | chr7: 27190275-27191115 |
| chr2: 177001222-177001783 | chr4: 154170369-154170600 | chr7: 27194584-27194827 |
| chr2: 177005365-177005666 | chr4: 154605087-154606052 | chr7: 27208872-27209616 |
| chr2: 177029414-177029941 | chr4: 156588298-156589292 | chr7: 27212417-27214396 |
| chr2: 177052958-177054350 | chr4: 15704641-15705000 | chr7: 27219310-27219750 |
| chr2: 177502241-177502910 | chr4: 157892686-157893286 | chr7: 27224268-27224596 |
| chr2: 180725718-180726465 | chr4: 157997167-157997686 | chr7: 27275056-27275626 |
| chr2: 183902403-183903625 | chr4: 166299976-166300668 | chr7: 29603110-29603328 |
| chr2: 185463209-185463589 | chr4: 166794546-166795341 | chr7: 30028519-30029822 |
| chr2: 189156417-189157719 | chr4: 168155006-168155765 | chr7: 31232678-31232900 |
| chr2: 191044980-191045829 | chr4: 170946767-170947925 | chr7: 35301151-35301731 |
| chr2: 192109829-192110940 | chr4: 171010944-171011503 | chr7: 37487355-37488672 |
| chr2: 197457393-197458522 | chr4: 175750331-175750584 | chr7: 37960317-37961046 |
| chr2: 200320503-200329681 | chr4: 176922474-176922820 | chr7: 38670440-38671008 |
| chr2: 201450527-201451027 | chr4: 176923076-176923630 | chr7: 42276004-42277850 |
| chr2: 201983199-201983709 | chr4: 176986922-176987360 | chr7: 42533077-42533283 |
| chr2: 207308267-207308907 | chr4: 183369277-183370459 | chr7: 44079718-44080568 |
| chr2: 20865290-20867589 | chr4: 184319565-184320198 | chr7: 44364433-44365328 |
| chr2: 21022565-21022934 | chr4: 184643915-184644414 | chr7: 48128047-48128499 |
| chr2: 213402181-213403867 | chr4: 186455984-186456864 | chr7: 5111621-5112088 |
| chr2: 214148958-214149426 | chr4: 187476280-187476844 | chr7: 51383312-51384938 |
| chr2: 217236026-217237129 | chr4: 190942735-190944898 | chr7: 5336514-5336894 |
| chr2: 217497812-217498847 | chr4: 21950086-21950346 | chr7: 54731780-54732479 |
| chr2: 217556803-217557174 | chr4: 26030421-26030692 | chr7: 55000877-55001135 |
| chr2: 218621280-218621498 | chr4: 2765639-2766063 | chr7: 56183578-56184165 |
| chr2: 219156978-219157380 | chr4: 298804-299312 | chr7: 63767751-63767991 |
| chr2: 219252422-219252631 | chr4: 30718854-30719847 | chr7: 64349425-64350151 |
| chr2: 219748277-219748805 | chr4: 30721205-30724842 | chr7: 64407590-64408299 |
| chr2: 219762988-219763537 | chr4: 330163-332068 | chr7: 65037625-65037864 |
| chr2: 219773547-219774103 | chr4: 3464800-3465440 | chr7: 65509075-65509594 |
| chr2: 219827660-219828205 | chr4: 379900-380399 | chr7: 65878464-65878977 |
| chr2: 219866132-219868054 | chr4: 38869402-38869780 | chr7: 6654746-6655860 |
| chr2: 219922155-219923130 | chr4: 4108735-4109499 | chr7: 69062375-69065037 |
| chr2: 219924820-219926130 | chr4: 41218315-41218768 | chr7: 70060835-70061456 |
| chr2: 220159439-220159977 | chr4: 41646293-41646582 | chr7: 73021487-73021720 |
| chr2: 220196432-220196768 | chr4: 41753670-41753947 | chr7: 73867788-73868061 |
| chr2: 220348956-220349784 | chr4: 42399153-42400802 | chr7: 74202334-74203629 |
| chr2: 220361463-220363254 | chr4: 4577005-4577448 | chr7: 74572475-74573770 |
| chr2: 220377744-220377946 | chr4: 46391904-46392572 | chr7: 75889087-75889345 |
| [symbol] 64-220406840 | chr4: 48271363-48271846 | chr7: 75889498-75889760 |
| chr2: 223288923-223290013 | chr4: 48272027-48272298 | chr7: 75896511-75896944 |
| chr2: 223536123-223536565 | chr4: 48492118-48493589 | chr7: 76026791-76027223 |
| chr2: 224701963-224702790 | chr4: 4854614-4855210 | chr7: 77649015-77649543 |
| chr2: 225906654-225907464 | chr4: 4873263-4873613 | chr7: 82072022-82073520 |
| chr2: 228582486-228582821 | chr4: 48908293-48908850 | chr7: 82791675-82792412 |
| chr2: 228736231-228736544 | chr4: 52917389-52918280 | chr7: 8473140-8475199 |
| chr2: 229045958-229046553 | chr4: 53617119-53617607 | chr7: 84814840-84816242 |
| chr2: 230135709-230136004 | chr4: 55991403-55992171 | chr7: 852961-853230 |
| chr2: 231692698-231693496 | chr4: 5713036-5713451 | chr7: 87256959-87258444 |
| chr2: 231712776-231712982 | chr4: 57687611-57687906 | chr7: 88388489-88389283 |
| chr2: 231902027-231903122 | chr4: 62065884-62068801 | chr7: 90225083-90226364 |
| chr2: 232276743-232277135 | chr4: 667274-667601 | chr7: 915754-916644 |
| chr2: 232765247-232765491 | chr4: 7194538-7195467 | chr7: 94536850-94537477 |
| chr2: 232791576-232792016 | chr4: 74486045-74486258 | chr7: 95025560-95026122 |
| chr2: 233367781-233368577 | chr4: 74702421-74702627 | chr7: 96631384-96631800 |
| chr2: 233740669-233741879 | chr4: 74809870-74810089 | chr7: 96746768-96747190 |
| chr2: 234776883-234777098 | chr4: 74864114-74864329 | chr7: 98971650-98973170 |
| chr2: 236578081-236580153 | chr4: 74964657-74965279 | chr7: 993402-994574 |
| chr2: 238395062-238396241 | chr4: 77507078-77507339 | chr7: 99768885-99769559 |
| chr2: 238599858-238601430 | chr4: 77610325-77610943 | chr8: 102092795-102093240 |
| chr2: 238768074-238768831 | chr4: 786119-786341 | chr8: 103822615-103823263 |
| chr2: 239755097-239758310 | chr4: 7940564-7941853 | chr8: 104310810-104311620 |
| chr2: 241391877-241393598 | chr4: 79472807-79473177 | chr8: 104383410-104384109 |
| chr2: 241395249-241395802 | chr4: 81123509-81124318 | chr8: 104510871-104513913 |
| chr2: 241771656-241772194 | chr4: 81124469-81124845 | chr8: 105235385-105235979 |
| chr2: 241855375-241855631 | chr4: 81951942-81952808 | chr8: 105600454-105601704 |
| chr2: 241922778-241923143 | chr4: 8594413-8594777 | chr8: 10586614-10586886 |
| chr2: 242498014-242499274 | chr4: 87515184-87515735 | chr8: 107669778-107670591 |
| chr2: 242742599-242743758 | chr4: 8869045-8870067 | chr8: 110374553-110374793 |
| chr2: 242785912-242786616 | chr4: 8893372-8896268 | chr8: 11058625-11058844 |
| chr2: 242808406-242808618 | chr4: 89378224-89378948 | chr8: 11059012-11059242 |
| chr2: 242823447-242823970 | chr4: 91048412-91049682 | chr8: 110986114-110986983 |
| chr2: 24300061-24300294 | chr4: 95678799-95679809 | chr8: 11555100-11555603 |
| chr2: 26396103-26397204 | chr5: 102201584-102201912 | chr8: 11759863-11760212 |
| chr2: 26401696-26402099 | chr5: 10307521-10307913 | chr8: 119963947-119964178 |
| chr2: 26407423-26408253 | chr5: 112630220-112630624 | chr8: 119964390-119964637 |
| chr2: 26726190-26726710 | chr5: 112823257-112824304 | chr8: 120220389-120221222 |
| chr2: 26785115-26785687 | chr5: 113391072-113392005 | chr8: 12990091-12990914 |
| chr2: 27341568-27341923 | chr5: 113696517-113699195 | chr8: 13133843-13134312 |
| chr2: 27958208-27958723 | chr5: 115151349-115152713 | chr8: 131455249-131456285 |
| [symbol] 2-31361821 | chr5: 11903551-11904703 | chr8: 133492399-133493586 |
| chr2: 31456741-31457653 | chr5: 121412501-121414077 | chr8: 142318354-142319155 |
| chr2: 38763115-38763564 | chr5: 121647451-121648147 | chr8: 143530646-143530872 |
| chr2: 42068364-42068594 | chr5: 122424906-122425958 | chr8: 143531119-143534495 |
| chr2: 43864323-43864956 | chr5: 122426126-122426836 | chr8: 143556715-143556997 |
| chr2: 45161428-45163058 | chr5: 122433879-122435551 | chr8: 144099457-144099731 |
| chr2: 45235512-45237792 | chr5: 125930602-125931049 | chr8: 144503421-144503808 |
| chr2: 47499465-47499813 | chr5: 126564998-126565580 | chr8: 144640397-144640772 |
| chr2: 47796924-47799166 | chr5: 127872564-127874945 | chr8: 144798488-144799038 |
| chr2: 48757212-48757785 | chr5: 128300801-128301329 | chr8: 144822012-144822805 |
| chr2: 54785027-54785969 | chr5: 128795504-128797417 | chr8: 144842965-144843542 |
| chr2: 56410866-56412539 | chr5: 131346893-131347776 | chr8: 145555343-145562310 |
| chr2: 58273643-58274422 | chr5: 132155289-132155497 | chr8: 145697880-145698784 |
| chr2: 64836207-64836765 | chr5: 132946745-132948467 | chr8: 145700239-145700822 |
| chr2: 65086805-65087180 | chr5: 134824932-134825224 | chr8: 145806259-145806713 |
| chr2: 66660453-66660794 | chr5: 134827286-134827644 | chr8: 145909677-145912846 |
| chr2: 66661119-66661657 | chr5: 135170272-135171156 | chr8: 15397637-15398287 |
| chr2: 68870352-68871055 | chr5: 135527057-135528978 | chr8: 16884364-16885339 |
| chr2: 69240084-69241004 | chr5: 139017134-139017668 | chr8: 17354322-17355250 |
| chr2: 70351316-70351550 | chr5: 139040820-139041028 | chr8: 1771362-1772760 |
| chr2: 7057531-7058433 | chr5: 139047906-139048235 | chr8: 22089368-22089668 |
| chr2: 70994448-70995385 | chr5: 139076543-139077179 | chr8: 22408673-22409635 |
| chr2: 71192095-71192495 | chr5: 139081002-139081202 | chr8: 22456092-22456508 |
| chr2: 71503548-71504233 | chr5: 139135590-139135976 | chr8: 22457124-22457753 |
| chr2: 71680559-71681276 | chr5: 139174905-139176172 | chr8: 22960385-22960927 |
| chr2: 71693165-71694127 | chr5: 139227606-139228279 | chr8: 23081957-23082975 |
| chr2: 73151201-73152060 | chr5: 139742900-139743148 | chr8: 23260443-23261867 |
| chr2: 74347274-74347777 | chr5: 139927158-139927491 | chr8: 24770909-24772547 |
| chr2: 74425445-74426423 | chr5: 140011483-140012739 | chr8: 25897201-25897612 |
| chr2: 74725040-74727038 | chr5: 140167092-140168139 | chr8: 26371173-26372830 |
| chr2: 74776017-74776897 | chr5: 140207726-140208078 | chr8: 27183091-27183390 |
| chr2: 74781495-74782685 | chr5: 140261885-140262153 | chr8: 27348659-27348883 |
| chr2: 75147006-75147336 | chr5: 140531158-140532017 | chr8: 29210484-29210801 |
| chr2: 75426593-75428030 | chr5: 140568217-140569118 | chr8: 33371898-33372526 |
| chr2: 7571072-7571642 | chr5: 140573424-140574316 | chr8: 35092680-35093559 |
| chr2: 75787718-75788312 | chr5: 140604454-140605304 | chr8: 35093901-35094111 |
| chr2: 79220193-79220592 | chr5: 140615392-140616353 | chr8: 38325092-38326374 |
| chr2: 79739697-79740243 | chr5: 140620865-140621698 | chr8: 38644474-38645760 |
| chr2: 85359957-85362593 | chr5: 140741175-140741738 | chr8: 40755165-40755404 |
| chr2: 85640970-85641259 | chr5: 140750051-140750264 | chr8: 41624556-41625380 |
| chr2: 85811341-85811855 | chr5: 140762402-140762768 | chr8: 41654876-41655984 |
| [symbol] 0-85982198 | chr5: 140767197-140767695 | chr8: 49231523-49231810 |
| chr2: 8818293-8818503 | chr5: 140777443-140777938 | chr8: 494156-496083 |
| chr2: 88316234-88316859 | chr5: 140802400-140802832 | chr8: 49647703-49647988 |
| chr2: 88469692-88470386 | chr5: 141132812-141133338 | chr8: 54569668-54570010 |
| chr2: 96054895-96055143 | chr5: 141228931-141230084 | chr8: 54789582-54790665 |
| chr2: 96192056-96193072 | chr5: 141705392-141705688 | chr8: 54791846-54795141 |
| chr2: 96314996-96315230 | chr5: 142782072-142785071 | chr8: 57232371-57232795 |
| chr2: 98703355-98703889 | chr5: 145316146-145316354 | chr8: 58907002-58907822 |
| chr20: 1206680-1207119 | chr5: 146257347-146258575 | chr8: 60030135-60032356 |
| chr20: 13200671-13202616 | chr5: 146614284-146614560 | chr8: 61193313-61194195 |
| chr20: 13975769-13976287 | chr5: 149681703-149682601 | chr8: 65491084-65491319 |
| chr20: 13976701-13977068 | chr5: 150004671-150004984 | chr8: 65492936-65494452 |
| chr20: 18039533-18039928 | chr5: 150284386-150284635 | chr8: 6691834-6693135 |
| chr20: 1874934-1875718 | chr5: 150325905-150326194 | chr8: 67089250-67089962 |
| chr20: 19738040-19739773 | chr5: 150537020-150537418 | chr8: 6949350-6950039 |
| chr20: 22562737-22566104 | chr5: 151304227-151304824 | chr8: 70946891-70947299 |
| chr20: 23028404-23032218 | chr5: 153853057-153853551 | chr8: 72917306-72917641 |
| chr20: 24898955-24899181 | chr5: 153862143-153862451 | chr8: 74005022-74005856 |
| chr20: 30196119-30196585 | chr5: 154026819-154027257 | chr8: 7543874-7544385 |
| chr20: 30582789-30583099 | chr5: 15500077-15500923 | chr8: 80695831-80696458 |
| chr20: 30605745-30605970 | chr5: 155107505-155108934 | chr8: 81805956-81806327 |
| chr20: 3229239-3229693 | chr5: 157002175-157003182 | chr8: 82192379-82193685 |
| chr20: 32450501-32450765 | chr5: 157285770-157286254 | chr8: 82644604-82644849 |
| chr20: 33865768-33866091 | chr5: 158758475-158758839 | chr8: 8820767-8821112 |
| chr20: 3387966-3388901 | chr5: 159343216-159343574 | chr8: 89339966-89340481 |
| chr20: 34188720-34190267 | chr5: 159738782-159739913 | chr8: 95651207-95651760 |
| chr20: 36793550-36793867 | chr5: 160974693-160975384 | chr8: 95652456-95652873 |
| chr20: 37230524-37230742 | chr5: 16179065-16180420 | chr8: 95653899-95654733 |
| chr20: 37274692-37275134 | chr5: 168727430-168728275 | chr8: 97156764-97158030 |
| chr20: 39311379-39312435 | chr5: 170288880-170289737 | chr8: 97505748-97507607 |
| chr20: 39316551-39319987 | chr5: 170877799-170878210 | chr8: 9756178-9756993 |
| chr20: 4202149-4202765 | chr5: 172068287-172069174 | chr8: 99305904-99306726 |
| chr20: 42285962-42286535 | chr5: 172385523-172385912 | chr9: 103173890-103174153 |
| chr20: 43438738-43439546 | chr5: 172710766-172711062 | chr9: 103790613-103791764 |
| chr20: 44098281-44099536 | chr5: 172754057-172757098 | chr9: 103791945-103792173 |
| chr20: 44452577-44453162 | chr5: 174151479-174152364 | chr9: 104248248-104249501 |
| chr20: 44539730-44540099 | chr5: 175223610-175224679 | chr9: 1051820-1052240 |
| chr20: 44642095-44642406 | chr5: 175621334-175621827 | chr9: 10612636-10613333 |
| chr20: 44746823-44747060 | chr5: 176236762-176238081 | chr9: 107509907-107510768 |
| chr20: 44935933-44937310 | chr5: 176789979-176790296 | chr9: 110249749-110252660 |
| chr20: 45142001-45142337 | chr5: 176830276-176831639 | chr9: 112262011-112262317 |
| [symbol] 51-45524020 | chr5: 177098635-177099525 | chr9: 112402768-112403349 |
| chr20: 47443735-47445181 | chr5: 177366539-177366973 | chr9: 114287381-114287695 |
| chr20: 48598960-48599657 | chr5: 177433282-177434067 | chr9: 116111664-116112189 |
| chr20: 50158905-50159509 | chr5: 177540208-177541234 | chr9: 116450146-116450454 |
| chr20: 55500348-55501102 | chr5: 178016559-178017670 | chr9: 116860474-116860695 |
| chr20: 55839288-55839766 | chr5: 178322714-178323538 | chr9: 123631107-123631672 |
| chr20: 55840217-55841794 | chr5: 178367621-178368725 | chr9: 123690772-123691675 |
| chr20: 55964273-55964656 | chr5: 178770725-178772794 | chr9: 124061806-124062229 |
| chr20: 55964917-55965271 | chr5: 180479586-180480959 | chr9: 124461798-124462190 |
| chr20: 56323974-56324254 | chr5: 180542154-180542402 | chr9: 124498514-124498962 |
| chr20: 56725858-56726113 | chr5: 2038528-2038949 | chr9: 124975754-124976692 |
| chr20: 57224696-57226322 | chr5: 31855004-31855426 | chr9: 125109008-125109644 |
| chr20: 57581903-57582595 | chr5: 36690208-36690658 | chr9: 126135408-126136193 |
| chr20: 57797224-57797441 | chr5: 373843-374426 | chr9: 126762469-126762683 |
| chr20: 59826978-59828978 | chr5: 38556223-38557563 | chr9: 126807511-126808181 |
| chr20: 6103437-6103970 | chr5: 38845503-38846476 | chr9: 129677707-129678009 |
| chr20: 61147458-61147787 | chr5: 41510325-41510651 | chr9: 130461544-130461839 |
| chr20: 61200973-61201272 | chr5: 42423531-42423740 | chr9: 131012455-131013429 |
| chr20: 61456340-61456565 | chr5: 42424339-42425047 | chr9: 131965038-131965636 |
| chr20: 61884645-61886387 | chr5: 42994627-42994936 | chr9: 132020630-132021038 |
| chr20: 61927195-61927482 | chr5: 42995123-42995415 | chr9: 132082872-132083582 |
| chr20: 61937483-61937738 | chr5: 43017969-43018668 | chr9: 132099124-132099616 |
| chr20: 61992187-61993599 | chr5: 43040346-43040633 | chr9: 132145577-132146328 |
| chr20: 62600654-62601676 | chr5: 43040846-43041161 | chr9: 132331219-132331458 |
| chr20: 62673793-62674131 | chr5: 43396898-43397364 | chr9: 132359673-132360061 |
| chr20: 62714764-62715761 | chr5: 472601-474261 | chr9: 132382433-132383004 |
| chr20: 62958974-62959513 | chr5: 474959-475319 | chr9: 132499969-132500553 |
| chr20: 708602-709290 | chr5: 49736608-49737300 | chr9: 13278313-13279805 |
| chr20: 8112885-8113592 | chr5: 55776605-55777233 | chr9: 132934214-132934483 |
| chr20: 9048959-9050018 | chr5: 57878726-57879177 | chr9: 133308594-133309448 |
| chr20: 9819272-9819861 | chr5: 58334837-58335881 | chr9: 133412891-133413096 |
| chr21: 18984536-18985697 | chr5: 60921535-60922472 | chr9: 134151854-134153015 |
| chr21: 27011625-27012398 | chr5: 6448754-6449629 | chr9: 134158161-134158682 |
| chr21: 28216559-28218117 | chr5: 66299769-66300083 | chr9: 136451013-136451276 |
| chr21: 32929928-32932017 | chr5: 67584214-67584451 | chr9: 137217063-137218078 |
| chr21: 36041306-36043224 | chr5: 68710808-68711520 | chr9: 137299191-137299437 |
| chr21: 38119794-38120742 | chr5: 691081-691376 | chr9: 137533360-137534397 |
| chr21: 38352857-38353274 | chr5: 72415612-72416766 | chr9: 138985838-138987846 |
| chr21: 38362016-38362868 | chr5: 72715408-72715997 | chr9: 139014622-139014848 |
| chr21: 40032244-40033665 | chr5: 72732366-72733732 | chr9: 139159210-139159560 |
| chr21: 40760627-40760829 | chr5: 74349801-74350239 | chr9: 139551255-139551559 |
| [symbol] 52-42880674 | chr5: 75378975-75380796 | chr9: 139552948-139553269 |
| chr21: 43373136-43374062 | chr5: 76011121-76012292 | chr9: 139553660-139553915 |
| chr21: 43917047-43917268 | chr5: 76115511-76116089 | chr9: 139595846-139596130 |
| chr21: 44073202-44074650 | chr5: 76941396-76941888 | chr9: 139872238-139873143 |
| chr21: 45148455-45149262 | chr5: 78365299-78365711 | chr9: 140051063-140051730 |
| chr21: 46129392-46129689 | chr5: 87437096-87437505 | chr9: 140317161-140318663 |
| chr21: 46351329-46352911 | chr5: 87976095-87976546 | chr9: 14348685-14349074 |
| chr21: 46706692-46707049 | chr5: 92906240-92908875 | chr9: 14349308-14349515 |
| chr22: 17849475-17850733 | chr5: 94619460-94621121 | chr9: 17134822-17135706 |
| chr22: 18923471-18923840 | chr5: 95170618-95170855 | chr9: 214587-215431 |
| chr22: 19753313-19755013 | chr5: 9544693-9546715 | chr9: 21559134-21559816 |
| chr22: 21319179-21319912 | chr5: 96038210-96038884 | chr9: 2241892-2242102 |
| chr22: 22862624-22863220 | chr6: 101841426-101841905 | chr9: 27528358-27528725 |
| chr9: 27528977-27529885 | | |
| chr9: 33044246-33044612 | | |
| chr9: 33447447-33447824 | | |
| chr9: 33750520-33751160 | | |
| chr9: 34377402-34377610 | | |
| chr9: 34379542-34380017 | | |
| chr9: 34577867-34578258 | | |
| chr9: 34589114-34591978 | | |
| chr9: 35756949-35757339 | | |
| chr9: 36036799-36037564 | | |
| chr9: 36258171-36258886 | | |
| chr9: 37575919-37576445 | | |
| chr9: 38069785-38069991 | | |
| chr9: 38423948-38424584 | | |
| chr9: 4297818-4300182 | | |
| chr9: 46148701-46149726 | | |
| chr9: 4662253-4662951 | | |
| chr9: 707022-707420 | | |
| chr9: 71788716-71789542 | | |
| chr9: 72658837-72659277 | | |
| chr9: 77502094-77502518 | | |
| chr9: 79073908-79074561 | | |
| chr9: 79520804-79521508 | | |
| chr9: 80911780-80912611 | | |
| chr9: 85677016-85678321 | | |
| chr9: 86571048-86572027 | | |
| chr9: 8857486-8858708 | | |
| chr9: 88713706-88714908 | | |
| [symbol] 5-89562647 | | |
| chr9: 90112515-90113817 | | |
| chr9: 90340716-90341542 | | |
| chr9: 90589210-90589807 | | |
| chr9: 93563776-93564546 | | |
| chr9: 93955501-93956420 | | |
| chr9: 94183408-94183994 | | |
| chr9: 95569430-95572255 | | |
| chr9: 95896008-95897016 | | |
| chr9: 97021465-97021967 | | |
| chr9: 97766650-97767955 | | |
| chr9: 97810766-97811272 | | |
| chr9: 99145525-99145849 | | |
| chr1: 10762450-10766925 | chr12: 101107864-101113622 | chr17: 48039283-48045064 |
| chr1: 110608266-110615303 | chr12: 103694091-103698418 | chr17: 48192635-48197085 |
| chr1: 113263574-113267787 | chr12: 104695349-104699984 | chr17: 48543571-48548900 |
| chr1: 113284333-113289172 | chr12: 106972413-106983086 | chr17: 4998370-5003205 |
| chr1: 114693137-114698672 | chr12: 113011100-113015529 | chr17: 50233176-50238466 |
| chr1: 115878168-115883332 | chr12: 113513165-113517970 | chr17: 59483574-59487780 |
| chr1: 116378360-116384364 | chr12: 113588807-113593304 | chr17: 59526980-59537254 |
| chr1: 1179757-1184470 | chr12: 113898751-113918717 | chr17: 6614423-6619471 |
| chr1: 119524783-119532712 | chr12: 114831912-114854360 | chr17: 6677206-6681710 |
| chr1: 119541057-119553320 | chr12: 114876144-114888579 | chr17: 70109980-70122442 |
| chr1: 12121489-12126148 | chr12: 115107504-115112061 | chr17: 71946479-71951255 |
| chr1: 145073484-145077845 | chr12: 117796077-117801448 | chr17: 72853622-72860012 |
| chr1: 146550329-146554577 | chr12: 119210111-119214393 | chr17: 72913569-72918510 |
| chr1: 1468605-1477220 | chr12: 120833587-120837927 | chr17: 73747619-73752178 |
| chr1: 147780067-147784473 | chr12: 122014171-122019693 | chr17: 74015770-74020658 |
| chr1: 149330994-149335389 | chr12: 123752050-123756373 | chr17: 74531282-74536566 |
| chr1: 155145186-155149444 | chr12: 127208779-127213651 | chr17: 75240872-75254180 |
| chr1: 155262319-155267536 | chr12: 127938452-127942907 | chr17: 75275318-75280172 |
| chr1: 155288607-155293001 | chr12: 129335871-129340653 | chr17: 75366689-75372506 |
| chr1: 156103708-156108171 | chr12: 130385610-130391139 | chr17: 75396285-75400527 |
| chr1: 156336759-156341251 | chr12: 130906778-130911191 | chr17: 75445478-75449821 |
| chr1: 156356051-156360252 | chr12: 131197825-131202157 | chr17: 77803867-77811046 |
| chr1: 156388404-156393581 | chr12: 132903450-132908206 | chr17: 7830533-7835164 |
| chr1: 156861416-156865711 | chr12: 14132627-14137242 | chr17: 78997641-79001641 |
| [symbol] 05-160342843 | chr12: 15473319-15477901 | chr17: 7903928-7909445 |
| chr1: 161693638-161699298 | chr12: 184864-189610 | chr17: 79312963-79322653 |
| chr1: 164543541-164547917 | chr12: 29300035-29304954 | chr17: 79857809-79862963 |
| chr1: 165321704-165328328 | chr12: 3306813-3312270 | chr17: 932418-937088 |
| chr1: 16858874-16864296 | chr12: 3473011-3477654 | chr18: 11146308-11151936 |
| chr1: 170628457-170632851 | chr12: 41084523-41089102 | chr18: 11748954-11754756 |
| chr1: 173636663-173641045 | chr12: 45442203-45447386 | chr18: 12252148-12257089 |
| chr1: 175566377-175570808 | chr12: 48397169-48401372 | chr18: 13639585-13644415 |
| chr1: 177131393-177135846 | chr12: 49181050-49185282 | chr18: 13866533-13871026 |
| chr1: 179542721-179547307 | chr12: 49369691-49377550 | chr18: 19742937-19754363 |
| chr1: 180196120-180206975 | chr12: 49482921-49487178 | chr18: 30347691-30354302 |
| chr1: 181285301-181289873 | chr12: 5016586-5023171 | chr18: 35142908-35149628 |
| chr1: 181450707-181455073 | chr12: 5151013-5156346 | chr18: 43606141-43610510 |
| chr1: 18434552-18439673 | chr12: 52113411-52117679 | chr18: 44334184-44340100 |
| chr1: 18954896-18970739 | chr12: 52406382-52410675 | chr18: 44770993-44780084 |
| chr1: 19201875-19206234 | chr12: 52650019-52654743 | chr18: 44787407-44792678 |
| chr1: 197885089-197889791 | chr12: 53105913-53110471 | chr18: 54786960-54791194 |
| chr1: 200007808-200012036 | chr12: 53357193-53361507 | chr18: 55017708-55023605 |
| chr1: 201250453-201255648 | chr12: 53489573-53493955 | chr18: 55092826-55110853 |
| chr1: 202160959-202165390 | chr12: 54069054-54073265 | chr18: 55920988-55926068 |
| chr1: 202676882-202681769 | chr12: 54319302-54323721 | chr18: 56885092-56889665 |
| chr1: 203042723-203047390 | chr12: 54336762-54341168 | chr18: 56937625-56943540 |
| chr1: 208130328-208135117 | chr12: 54352530-54382102 | chr18: 58998684-59003692 |
| chr1: 214151215-214161080 | chr12: 54421428-54428709 | chr18: 61141927-61145927 |
| chr1: 21614381-21619101 | chr12: 54438643-54450091 | chr18: 70531966-70538871 |
| chr1: 217308750-217313178 | chr12: 54517769-54522457 | chr18: 72914108-72919233 |
| chr1: 221048449-221070185 | chr12: 57616770-57621402 | chr18: 73165403-73169920 |
| chr1: 225863069-225867328 | chr12: 58001881-58006249 | chr18: 74151240-74157073 |
| chr1: 226073151-226077680 | chr12: 58156856-58162000 | chr18: 74797145-74802038 |
| chr1: 226125113-226129695 | chr12: 63541637-63546967 | chr18: 74959557-74965822 |
| chr1: 228783987-228788204 | chr12: 6436273-6440931 | chr18: 76730971-76743244 |
| chr1: 231294560-231299345 | chr12: 65216246-65221143 | chr18: 77545966-77560948 |
| chr1: 24227116-24231537 | chr12: 65512879-65517863 | chr18: 902579-911574 |
| chr1: 243644395-243648888 | chr12: 72663684-72669551 | chr19: 10404935-10409342 |
| chr1: 248018331-248023252 | chr12: 75600992-75605344 | chr19: 10461627-10466378 |
| chr1: 25253528-25261005 | chr12: 81100035-81104716 | chr19: 1061545-1066265 |
| chr1: 2770127-2774665 | chr12: 81469570-81474119 | chr19: 1106395-1111610 |
| chr1: 29583898-29588598 | chr12: 99137387-99141769 | chr19: 11592373-11596987 |
| chr1: 2977276-2982758 | chr13: 100545634-100550911 | chr19: 12664244-12668682 |
| chr1: 32050472-32054771 | chr13: 100639335-100644188 | chr19: 12765750-12769980 |
| chr1: 34626784-34632976 | chr13: 102566426-102571495 | chr19: 12829794-12834225 |
| [symbol] 3-34645024 | chr13: 108516335-108521063 | chr19: 12878575-12882888 |
| chr1: 36547555-36551965 | chr13: 109145799-109151019 | chr19: 13122960-13127259 |
| chr1: 38217703-38222012 | chr13: 112705805-112730419 | chr19: 13133318-13138169 |
| chr1: 38459585-38463988 | chr13: 112756599-112763113 | chr19: 13196700-13200999 |
| chr1: 38939920-38944404 | chr13: 20873519-20878214 | chr19: 13211451-13215821 |
| chr1: 39042060-39046561 | chr13: 27332227-27337205 | chr19: 13614753-13619267 |
| chr1: 39978366-39983768 | chr13: 28364550-28370505 | chr19: 14087571-14091796 |
| chr1: 40233768-40239190 | chr13: 28496227-28501046 | chr19: 15290400-15294632 |
| chr1: 40767187-40771871 | chr13: 28547840-28552246 | chr19: 1746168-1752243 |
| chr1: 41282848-41287149 | chr13: 32887117-32892116 | chr19: 18977352-18983200 |
| chr1: 41829977-41834542 | chr13: 36042845-36055119 | chr19: 19366709-19374393 |
| chr1: 44029287-44033853 | chr13: 51415372-51420149 | chr19: 21767190-21771786 |
| chr1: 46949169-46953792 | chr13: 53417898-53424872 | chr19: 2422006-2429983 |
| chr1: 47007576-47012132 | chr13: 58201587-58210930 | chr19: 30713550-30719970 |
| chr1: 4711990-4718555 | chr13: 79179945-79185880 | chr19: 33623468-33627805 |
| chr1: 47907713-47913020 | chr13: 84451665-84455897 | chr19: 35631410-35635697 |
| chr1: 50878917-50884103 | chr13: 93877246-93882877 | chr19: 36244329-36249982 |
| chr1: 50890438-50895243 | chr14: 101190852-101195499 | chr19: 36334276-36339138 |
| chr1: 53525573-53530974 | chr14: 101921576-101927995 | chr19: 36498170-36502530 |
| chr1: 53740298-53744845 | chr14: 103653242-103657928 | chr19: 36521392-36525887 |
| chr1: 55503061-55508015 | chr14: 105165664-105170129 | chr19: 3866587-3871217 |
| chr1: 61513876-61518831 | chr14: 24042887-24048760 | chr19: 38698334-38702577 |
| chr1: 63780395-63798140 | chr14: 24639054-24644220 | chr19: 38874071-38878332 |
| chr1: 65729412-65733849 | chr14: 24801679-24806353 | chr19: 39735690-39741288 |
| chr1: 65989002-65993811 | chr14: 29234836-29239832 | chr19: 39752974-39758540 |
| chr1: 66256441-66260918 | chr14: 29252366-29257069 | chr19: 40312927-40317144 |
| chr1: 67216080-67220293 | chr14: 33400095-33406079 | chr19: 405012-411511 |
| chr1: 67771330-67775767 | chr14: 36971170-36996488 | chr19: 42889312-42893646 |
| chr1: 77745315-77750224 | chr14: 37047334-37055690 | chr19: 44201559-44205987 |
| chr1: 86619279-86624871 | chr14: 37114189-37138348 | chr19: 44276274-44280777 |
| chr1: 91170103-91194804 | chr14: 38676246-38682937 | chr19: 45258353-45263809 |
| chr1: 91298980-91303891 | chr14: 38722255-38727537 | chr19: 45896880-45902315 |
| chr1: 92943908-92954609 | chr14: 48141434-48147589 | chr19: 45999831-46004686 |
| chr10: 100990157-100994687 | chr14: 51336713-51341146 | chr19: 46316491-46321266 |
| chr10: 101277942-101292338 | chr14: 52732208-52737486 | chr19: 46913312-46917802 |
| chr10: 102277163-102281730 | chr14: 54416678-54420881 | chr19: 47149769-47155125 |
| chr10: 102417148-102421668 | chr14: 57258879-57286558 | chr19: 48963003-48967792 |
| chr10: 102471207-102493011 | chr14: 58329677-58335121 | chr19: 49667276-49671552 |
| chr10: 102505483-102511646 | chr14: 60971773-60980180 | chr19: 50879419-50883664 |
| chr10: 102889011-102908693 | chr14: 61101979-61106663 | chr19: 50929271-50933638 |
| chr10: 102973970-102980096 | chr14: 62277477-62282019 | chr19: 51167660-51174023 |
| [symbol] 035-102998646 | chr14: 69254677-69259036 | chr19: 51599823-51604260 |
| chr10: 103041991-103046480 | chr14: 74704189-74710192 | chr19: 51813158-51817458 |
| chr10: 105359785-105364188 | chr14: 77734734-77739772 | chr19: 54410711-54415087 |
| chr10: 105418686-105423076 | chr14: 85995469-86002478 | chr19: 54479413-54485572 |
| chr10: 105525044-105529044 | chr14: 92787495-92792712 | chr19: 55595978-55600887 |
| chr10: 106397568-106404812 | chr14: 95235623-95241679 | chr19: 55813941-55818277 |
| chr10: 108921781-108926805 | chr14: 95824676-95828941 | chr19: 56596039-56602296 |
| chr10: 109672197-109676964 | chr15: 100911439-100916022 | chr19: 56986314-56991741 |
| chr10: 110669725-110674326 | chr15: 23155795-23160624 | chr19: 58092740-58097764 |
| chr10: 111214605-111219083 | chr15: 27110031-27115479 | chr19: 5827049-5831474 |
| chr10: 118028733-118036230 | chr15: 27213952-27218856 | chr19: 58543116-58556587 |
| chr10: 118890162-118902329 | chr15: 33007531-33013696 | chr19: 7931264-7936898 |
| chr10: 118998436-119003530 | chr15: 33600817-33606003 | chr19: 8672333-8676764 |
| chr10: 119309205-119315563 | chr15: 35044444-35049480 | chr19: 868775-873318 |
| chr10: 119492494-119496991 | chr15: 37388176-37392380 | chr2: 102801673-102806556 |
| chr10: 120351693-120357821 | chr15: 40266582-40271061 | chr2: 105457128-105482760 |
| chr10: 121575530-121580385 | chr15: 45406468-45411528 | chr2: 106679983-106684403 |
| chr10: 123920851-123925542 | chr15: 47474370-47479499 | chr2: 107101834-107106053 |
| chr10: 124899908-124913035 | chr15: 49252985-49257564 | chr2: 108600825-108605467 |
| chr10: 125423496-125428642 | chr15: 53074188-53089488 | chr2: 114031360-114038041 |
| chr10: 125648821-125653373 | chr15: 53095562-53100476 | chr2: 114254776-114260043 |
| chr10: 125730221-125734843 | chr15: 59155046-59159594 | chr2: 118979770-118984466 |
| chr10: 129532411-129539366 | chr15: 60285108-60300520 | chr2: 119590603-119618826 |
| chr10: 130336696-130340994 | chr15: 67071307-67075943 | chr2: 119912127-119918663 |
| chr10: 130506444-130510658 | chr15: 74417871-74425044 | chr2: 124780253-124785255 |
| chr10: 131262948-131267947 | chr15: 76628030-76635515 | chr2: 127411697-127416171 |
| chr10: 134595358-134604649 | chr15: 79572831-79577211 | chr2: 127780614-127784829 |
| chr10: 15759424-15764101 | chr15: 79722100-79727643 | chr2: 128419720-128424182 |
| chr10: 16559605-16565822 | chr15: 89145661-89151198 | chr2: 130761484-130765764 |
| chr10: 1776785-1782018 | chr15: 89310720-89315183 | chr2: 132180328-132185101 |
| chr10: 22621351-22636862 | chr15: 89901447-89924768 | chr2: 137520461-137525696 |
| chr10: 22762709-22769050 | chr15: 89947374-89955182 | chr2: 139535693-139540650 |
| chr10: 23459301-23465889 | chr15: 91640909-91645702 | chr2: 142885725-142890553 |
| chr10: 23478698-23484455 | chr15: 96871409-96879721 | chr2: 144692667-144697180 |
| chr10: 23981367-23986978 | chr15: 96893307-96912030 | chr2: 154725907-154731328 |
| chr10: 26502384-26509434 | chr15: 96957342-96962531 | chr2: 157183558-157188355 |
| chr10: 27545669-27550402 | chr16: 10910160-10914719 | chr2: 162271295-162286677 |
| chr10: 43426168-43431460 | chr16: 20082708-20087305 | chr2: 171669599-171682358 |
| chr10: 48436412-48441320 | chr16: 2226191-2232946 | chr2: 176929576-176984402 |
| chr10: 50600990-50608783 | chr16: 22822617-22828459 | chr2: 177010372-177027692 |
| chr10: 50815602-50822356 | chr16: 23722271-23726775 | chr2: 177034255-177045444 |
| [symbol] 96-63215009 | chr16: 24265041-24269527 | chr2: 182319762-182325029 |
| chr10: 71329450-71335392 | chr16: 3011017-3015228 | chr2: 182519222-182523927 |
| chr10: 75405414-75409706 | chr16: 3065522-3070358 | chr2: 19558964-19563650 |
| chr10: 76571196-76575507 | chr16: 31051480-31055800 | chr2: 198027069-198031438 |
| chr10: 8074003-8080378 | chr16: 3188766-3193389 | chr2: 200331688-200336172 |
| chr10: 88120925-88129364 | chr16: 3218439-3223356 | chr2: 207504775-207509422 |
| chr10: 94178316-94182754 | chr16: 48842552-48847264 | chr2: 20868007-20873280 |
| chr10: 94453525-94457896 | chr16: 49307124-49319263 | chr2: 219734133-219738788 |
| chr10: 94818027-94831040 | chr16: 49870450-49874926 | chr2: 219846292-219860917 |
| chr10: 99787615-99793320 | chr16: 51145491-51149944 | chr2: 220171871-220176283 |
| chr11: 105479127-105483422 | chr16: 51166267-51171110 | chr2: 220297484-220302243 |
| chr11: 115628399-115633117 | chr16: 51181700-51190763 | chr2: 220410342-220414678 |
| chr11: 119291321-119295943 | chr16: 54323041-54327703 | chr2: 223157726-223187468 |
| chr11: 123064518-123068986 | chr16: 54968302-54974846 | chr2: 233249362-233255414 |
| chr11: 124627724-124631926 | chr16: 55362824-55367483 | chr2: 237066072-237080762 |
| chr11: 128417199-128421513 | chr16: 55511221-55515526 | chr2: 238862316-238867170 |
| chr11: 128692085-128696688 | chr16: 58028215-58033633 | chr2: 241457633-241462047 |
| chr11: 131778329-131783532 | chr16: 6066915-6072401 | chr2: 241756142-241762783 |
| chr11: 132811563-132816395 | chr16: 62067122-62072634 | chr2: 25497764-25502429 |
| chr11: 132932060-132936291 | chr16: 66610750-66615412 | chr2: 30451567-30457655 |
| chr11: 132950539-132955307 | chr16: 67206068-67210678 | chr2: 31803294-31808403 |
| chr11: 133992710-133997090 | chr16: 67569253-67574728 | chr2: 3284325-3288530 |
| chr11: 14993129-14997908 | chr16: 67916680-67920909 | chr2: 3748829-3753927 |
| chr11: 17738790-17745779 | chr16: 68478865-68484822 | chr2: 38299277-38306518 |
| chr11: 20179201-20184325 | chr16: 71457782-71462338 | chr2: 45153196-45159049 |
| chr11: 20616198-20625399 | chr16: 82658652-82663813 | chr2: 45167506-45173884 |
| chr11: 27741473-27746564 | chr16: 84000270-84004860 | chr2: 45225645-45230783 |
| chr11: 2888389-2893337 | chr16: 86528748-86534994 | chr2: 45238373-45243579 |
| chr11: 31823744-31850776 | chr16: 86547070-86552512 | chr2: 45393870-45400186 |
| chr11: 32450145-32459311 | chr16: 86609389-86615821 | chr2: 465850-470659 |
| chr11: 36395927-36401398 | chr16: 88941428-88945669 | chr2: 50572046-50576817 |
| chr11: 43566922-43571854 | chr17: 1171536-1176733 | chr2: 54084777-54089266 |
| chr11: 44323658-44329932 | chr17: 12566668-12571335 | chr2: 5829188-5834208 |
| chr11: 46297545-46302216 | chr17: 12875271-12879773 | chr2: 63280515-63289097 |
| chr11: 46364877-46369101 | chr17: 14199727-14204052 | chr2: 66650692-66656218 |
| chr11: 60716429-60720888 | chr17: 14246392-14250721 | chr2: 66670432-66675636 |
| chr11: 61535001-61539001 | chr17: 15818621-15823325 | chr2: 66806569-66811404 |
| chr11: 624729-642628 | chr17: 1878790-1883116 | chr2: 71785431-71789897 |
| chr11: 64134815-64140187 | chr17: 19881326-19885610 | chr2: 73141056-73150260 |
| chr11: 64476844-64481598 | chr17: 21365115-21369592 | chr2: 80527678-80532846 |
| chr11: 64813041-64817722 | chr17: 27897512-27902067 | chr2: 80547579-80551798 |
| [symbol] 32-65355134 | chr17: 32482008-32486280 | chr2: 87013975-87020182 |
| chr11: 65407637-65412127 | chr17: 33774554-33778888 | chr2: 87086817-87091037 |
| chr11: 65814405-65818665 | chr17: 35289900-35302875 | chr2: 97190978-97195383 |
| chr11: 67348566-67353565 | chr17: 36715692-36720593 | chr20: 10196136-10200984 |
| chr11: 68620109-68624339 | chr17: 37319483-37324099 | chr20: 17204529-17210756 |
| chr11: 69515841-69521929 | chr17: 37761692-37766304 | chr20: 21374359-21380245 |
| chr11: 69829572-69834484 | chr17: 40935259-40939480 | chr20: 21483933-21498714 |
| chr11: 70209532-70213532 | chr17: 41275001-41280000 | chr20: 21684200-21697344 |
| chr11: 70506329-70510617 | chr17: 43035167-43039740 | chr20: 22546968-22561240 |
| chr11: 70670835-70675055 | chr17: 43470528-43476343 | chr20: 25061839-25067525 |
| chr11: 71950113-71954528 | chr17: 45947677-45951885 | chr20: 2537134-2541877 |
| chr11: 723597-728870 | chr17: 46602363-46610390 | chr20: 2727998-2733630 |
| chr11: 72530613-72535774 | chr17: 46618368-46634212 | chr20: 2778979-2783497 |
| chr11: 79146359-79154200 | chr17: 46667435-46676181 | chr20: 3143122-3147746 |
| chr11: 8188227-8192671 | chr17: 46689521-46699701 | chr20: 32854660-32859248 |
| chr11: 88239711-88244562 | chr17: 46794235-46802746 | chr20: 33294515-33300242 |
| chr11: 89222417-89226718 | chr17: 46822786-46827372 | chr20: 36010596-36015439 |
| chr20: 36224618-36228841 | chr6: 161186085-161190639 | chr20: 44655464-44661243 |
| chr20: 37350131-37359372 | chr6: 1617094-1623094 | |
| chr20: 39992546-39997810 | chr6: 166577974-166585423 | |
| chr20: 41815476-41821212 | chr6: 166664838-166669541 | |
| chr20: 44683772-44689610 | chr6: 170730120-170734442 | |
| chr20: 48182194-48186833 | chr6: 26612014-26616851 | |
| chr20: 51587708-51592020 | chr6: 27226101-27230364 | |
| chr20: 52787253-52792986 | chr6: 29593299-29597795 | |
| chr20: 5294267-5299798 | chr6: 29892141-29897117 | |
| chr20: 57087461-57092237 | chr6: 30093174-30097610 | |
| chr20: 57413136-57429047 | chr6: 30137719-30142263 | |
| chr20: 61701527-61706022 | chr6: 33046417-33050814 | |
| chr20: 688576-693099 | chr6: 33391593-33395908 | |
| chr20: 9494472-9498893 | chr6: 33653967-33658238 | |
| chr21: 19615099-19619874 | chr6: 35477389-35481678 | |
| chr21: 31309387-31314106 | chr6: 37614723-37619179 | |
| chr21: 32622145-32626382 | chr6: 38680950-38685265 | |
| chr21: 34393129-34402245 | chr6: 389189-395790 | |
| chr21: 38063180-38068185 | chr6: 4077053-4081443 | |
| chr21: 38074763-38083833 | chr6: 41526267-41530900 | |
| chr21: 42216490-42221222 | chr6: 41906746-41911711 | |
| chr22: 19744156-19748369 | chr6: 42070033-42074701 | |
| chr22: 19965280-19969808 | chr6: 42143848-42148053 | |
| chr22: 25079851-25084112 | chr6: 42877280-42881623 | |
| [symbol] 82-29714013 | chr6: 46653263-46658738 | |
| chr22: 31196492-31201033 | chr6: 50680335-50685214 | |
| chr22: 31498397-31503239 | chr6: 50785287-50793573 | |
| chr22: 37210770-37215467 | chr6: 50808643-50820431 | |
| chr22: 37463057-37467331 | chr6: 55037171-55041392 | |
| chr22: 37909980-37914258 | chr6: 5995028-6009797 | |
| chr22: 38377094-38381964 | chr6: 70990041-70994912 | |
| chr22: 38474837-38480839 | chr6: 7227878-7232865 | |
| chr22: 39260339-39265211 | chr6: 72296275-72300528 | |
| chr22: 42303618-42309254 | chr6: 78170232-78176088 | |
| chr22: 42320044-42324909 | chr6: 85470703-85476132 | |
| chr22: 42683895-42688095 | chr6: 99273764-99278038 | |
| chr22: 44255943-44260612 | chr6: 99288280-99292771 | |
| chr22: 44285498-44290061 | chr7: 100073304-100077551 | |
| chr22: 44724725-44729590 | chr7: 100815485-100825701 | |
| chr22: 46316694-46321087 | chr7: 101003900-101009443 | |
| chr22: 46438394-46443019 | chr7: 103083711-103088132 | |
| chr22: 48882885-48889043 | chr7: 103966784-103971959 | |
| chr22: 50494442-50499393 | chr7: 113722925-113729795 | |
| chr3: 11032447-11037384 | chr7: 12149221-12153559 | |
| chr3: 113158300-113162641 | chr7: 121938007-121959341 | |
| chr3: 121900743-121905645 | chr7: 124402175-124406432 | |
| chr3: 126111548-126115967 | chr7: 127988927-127994616 | |
| chr3: 127631994-127636588 | chr7: 128553330-128558650 | |
| chr3: 127792370-127798136 | chr7: 129418287-129425355 | |
| chr3: 12836472-12840782 | chr7: 130788359-130794773 | |
| chr3: 128717866-128723245 | chr7: 1360812-1365643 | |
| chr3: 129691128-129696841 | chr7: 136551855-136558194 | |
| chr3: 13112628-13117245 | chr7: 142492564-142497248 | |
| chr3: 133391119-133395657 | chr7: 143580126-143584610 | |
| chr3: 137480965-137493004 | chr7: 149387655-149391976 | |
| chr3: 138654628-138661107 | chr7: 149742403-149748469 | |
| chr3: 147106512-147116479 | chr7: 152619917-152624149 | |
| chr3: 147124989-147144391 | chr7: 153746408-153752444 | |
| chr3: 154144348-154148965 | chr7: 153999965-154004281 | |
| chr3: 157810054-157823836 | chr7: 155162558-155177248 | |
| chr3: 170301045-170305768 | chr7: 155239324-155245757 | |
| chr3: 172163373-172168738 | chr7: 155256828-155263403 | |
| chr3: 184054420-184058671 | chr7: 155300254-155305158 | |
| chr3: 185909345-185914228 | chr7: 155593693-155607095 | |
| chr3: 186076711-186082111 | chr7: 156407024-156411865 | |
| [symbol] 9-19192100 | chr7: 156793356-156803632 | |
| chr3: 192123822-192129994 | chr7: 156869055-156873297 | |
| chr3: 22411493-22416365 | chr7: 158934508-158940492 | |
| chr3: 236392-242140 | chr7: 19143873-19148256 | |
| chr3: 26662105-26666796 | chr7: 19182819-19187033 | |
| chr3: 27769639-27773942 | chr7: 20368004-20373504 | |
| chr3: 32859142-32863429 | chr7: 20828568-20832817 | |
| chr3: 3838514-3844772 | chr7: 23285222-23289508 | |
| chr3: 44061315-44065837 | chr7: 26413747-26418891 | |
| chr3: 44594536-44599018 | chr7: 27132098-27136736 | |
| chr3: 46616308-46620669 | chr7: 27144070-27150389 | |
| chr3: 49945622-49950430 | chr7: 27180614-27187562 | |
| chr3: 55506337-55510708 | chr7: 27195602-27208462 | |
| chr3: 62352292-62365082 | chr7: 27225521-27231043 | |
| chr3: 63261990-63266205 | chr7: 27258102-27262467 | |
| chr3: 64251534-64255819 | chr7: 27276946-27294197 | |
| chr3: 6900824-6906641 | chr7: 30719373-30724445 | |
| chr3: 71832069-71836653 | chr7: 32108064-32112910 | |
| chr3: 75665778-75671067 | chr7: 35294922-35300218 | |
| chr3: 75953760-75958308 | chr7: 37953623-37958555 | |
| chr3: 87839797-87844563 | chr7: 42265547-42269823 | |
| chr3: 9175692-9180189 | chr7: 43150021-43155340 | |
| chr4: 100868378-100873994 | chr7: 49811009-49817752 | |
| chr4: 105147-109898 | chr7: 53284852-53289192 | |
| chr4: 107954556-107959453 | chr7: 54610325-54614558 | |
| chr4: 109091039-109096546 | chr7: 56353509-56357798 | |
| chr4: 110220971-110226257 | chr7: 6588564-6592957 | |
| chr4: 111552966-111557504 | chr7: 6659876-6664695 | |
| chr4: 114898356-114902810 | chr7: 70594229-70600382 | |
| chr4: 122299568-122304290 | chr7: 71798758-71804768 | |
| chr4: 128542032-128546903 | chr7: 72836384-72840815 | |
| chr4: 134067163-134072442 | chr7: 73892816-73897110 | |
| chr4: 13522063-13528083 | chr7: 749713-754150 | |
| chr4: 140199065-140203449 | chr7: 87561343-87566571 | |
| chr4: 144618823-144624218 | chr7: 89745893-89751036 | |
| chr4: 147557206-147563901 | chr7: 90891568-90898683 | |
| chr4: 151502012-151507085 | chr7: 95223504-95228194 | |
| chr4: 154707513-154716240 | chr7: 96648222-96654246 | |
| chr4: 155661810-155666315 | chr7: 97359133-97365018 | |
| chr4: 156127169-156132209 | chr7: 97839637-97844005 | |
| chr4: 156678096-156683386 | chr8: 101115923-101120693 | |
| [symbol] 9-15782729 | chr8: 102502479-102506841 | |
| chr4: 158141297-158146053 | chr8: 105476673-105481340 | |
| chr4: 164262822-164267772 | chr8: 11534768-11540961 | |
| chr4: 169797087-169801625 | chr8: 11555853-11569212 | |
| chr4: 172731735-172737118 | chr8: 120426399-120431178 | |
| chr4: 174420025-174462054 | chr8: 130993922-130998149 | |
| chr4: 185935243-185944747 | chr8: 132050204-132056749 | |
| chr4: 187217321-187221745 | chr8: 139506796-139511774 | |
| chr4: 188914606-188918876 | chr8: 142526186-142531029 | |
| chr4: 190935926-190942591 | chr8: 143543446-143548178 | |
| chr4: 204378-208892 | chr8: 144806222-144812978 | |
| chr4: 24799110-24803902 | chr8: 144988271-145004135 | |
| chr4: 25088107-25092510 | chr8: 145101286-145110027 | |
| chr4: 41747185-41751811 | chr8: 145923411-145928101 | |
| chr4: 41867175-41884964 | chr8: 21642909-21649845 | |
| chr4: 46993129-46997872 | chr8: 21903462-21907757 | |
| chr4: 47032428-47036940 | chr8: 23560476-23569678 | |
| chr4: 4857633-4871173 | chr8: 24810947-24816299 | |
| chr4: 54964164-54978202 | chr8: 25898563-25907842 | |
| chr4: 5707986-5712495 | chr8: 26719643-26726566 | |
| chr4: 57519622-57524703 | chr8: 37820487-37826008 | |
| chr4: 5889204-5897116 | chr8: 41422342-41427300 | |
| chr4: 66533194-66537620 | chr8: 4846969-4854635 | |
| chr4: 680725-685079 | chr8: 49466684-49470959 | |
| chr4: 81107888-81121391 | chr8: 50820271-50824860 | |
| chr4: 85401831-85425190 | chr8: 53849702-53856426 | |
| chr4: 90226715-90231010 | chr8: 55364181-55382186 | |
| chr4: 93224349-93229007 | chr8: 57356127-57361415 | |
| chr4: 94753787-94758310 | chr8: 65279904-65292946 | |
| chr4: 959348-964155 | chr8: 65708991-65713722 | |
| chr5: 11382682-11387521 | chr8: 68862585-68866946 | |
| chr5: 115695135-115699589 | chr8: 70979874-70986888 | |
| chr5: 122428677-122433443 | chr8: 72466561-72471561 | |
| chr5: 134361093-134388370 | chr8: 85094760-85099247 | |
| chr5: 139136876-139141242 | chr8: 86348766-86353196 | |
| chr5: 140050060-140055381 | chr8: 87079654-87084046 | |
| chr5: 140303713-140309193 | chr8: 97167732-97174022 | |
| chr5: 140344106-140348931 | chr8: 9758751-9766748 | |
| chr5: 140785448-140790044 | chr8: 98287605-98292404 | |
| chr5: 140796758-140801359 | chr8: 99958498-99963438 | |
| chr5: 140808495-140814617 | chr8: 99982585-99988983 | |
| [symbol] 28-140866748 | chr9: 100608697-100622192 | |
| chr5: 145716290-145727852 | chr9: 102588743-102593303 | |
| chr5: 146886751-146891840 | chr9: 104497850-104503076 | |
| chr5: 148031473-148036080 | chr9: 112079403-112084905 | |
| chr5: 158476379-158480630 | chr9: 115820072-115825416 | |
| chr5: 158521907-158526598 | chr9: 120173254-120179496 | |
| chr5: 159397005-159401928 | chr9: 120505228-120509642 | |
| chr5: 170733170-170746107 | chr9: 122129087-122134214 | |
| chr5: 172108283-172113166 | chr9: 123654751-123658972 | |
| chr5: 172657050-172674971 | chr9: 124411513-124416193 | |
| chr5: 174156681-174161729 | chr9: 124985744-124993086 | |
| chr5: 175083005-175087756 | chr9: 126771247-126782953 | |
| chr5: 178419226-178424337 | chr9: 129370738-129391231 | |
| chr5: 179226284-179231003 | chr9: 131152347-131157923 | |
| chr5: 180484155-180488892 | chr9: 132457588-132462017 | |
| chr5: 1872908-1889743 | chr9: 133532535-133544394 | |
| chr5: 2736954-2759024 | chr9: 134427867-134432491 | |
| chr5: 31191953-31196419 | chr9: 135036714-135041978 | |
| chr5: 3588645-3605054 | chr9: 135453165-135468240 | |
| chr5: 37832672-37837128 | chr9: 136292739-136297236 | |
| chr5: 38255826-38261136 | chr9: 137965111-137969727 | |
| chr5: 45693395-45698510 | chr9: 139094666-139098993 | |
| chr5: 50683454-50688148 | chr9: 139394206-139399040 | |
| chr5: 52775789-52779996 | chr9: 139713664-139718441 | |
| chr5: 54517055-54529760 | chr9: 16724860-16729273 | |
| chr5: 59187047-59191894 | chr9: 17904420-17909488 | |
| chr5: 63253045-63259886 | chr9: 19786216-19791288 | |
| chr5: 71012918-71017715 | chr9: 21968914-21973190 | |
| chr5: 72524204-72531976 | chr9: 22003888-22008229 | |
| chr5: 72592148-72597808 | chr9: 23818692-23824135 | |
| chr5: 72674121-72680421 | chr9: 23848911-23853522 | |
| chr5: 76921888-76938984 | chr9: 32780937-32785625 | |
| chr5: 77138543-77149785 | chr9: 35842850-35846850 | |
| chr5: 77251833-77256049 | chr9: 36737535-36741782 | |
| chr5: 77266351-77270787 | chr9: 37000490-37004957 | |
| chr5: 77803754-77808313 | chr9: 77110713-77115927 | |
| chr5: 87966636-87972070 | chr9: 79631327-79640169 | |
| chr5: 87978879-87987810 | chr9: 86150354-86155777 | |
| chr5: 88183225-88187589 | chr9: 91790663-91795611 | |
| chr5: 92921488-92926497 | chr9: 95475297-95479708 | |
| chr5: 92937796-92942216 | chr9: 96106467-96110992 | |
| [symbol] 42-100041477 | chr9: 96708812-96720186 | |
| chr6: 100895008-100917245 | chr9: 967530-975276 | |
| chr6: 101844767-101849135 | chr9: 97399287-97404067 | |
| chr6: 10379559-10392565 | chr9: 98109365-98114362 | |
| chr6: 106427112-106436459 | chrX: 152610776-152615464 | |
| chr6: 108483672-108499996 | chrX: 67350651-67354923 | |
| chr6: 10879847-10884051 | chrX: 99889300-99893794 | |
| chr6: 110297366-110303267 | | |
| chr6: 117196090-117200705 | | |
| chr6: 117589534-117594279 | | |
| chr6: 117867098-117871530 | | |
| chr6: 127439554-127443760 | | |
| chr6: 134208640-134213218 | | |
| chr6: 134636798-134641021 | | |
| chr6: 137240316-137247442 | | |
| chr6: 1376446-1396170 | | |
| chr6: 137807343-137819223 | | |
| chr6: 138743349-138747593 | | |
| chr6: 150333526-150338278 | | |
| chr6: 150356873-150361394 | | |
| chr6: 154358587-154363008 | | |
| chr6: 168839439-168843699 | | |

[symbol]

indicates data missing or illegible when filed

1. A computer-implemented process for encoding a signal generated from processing a biological sample originating from a subject, the signal indicative of marker information in instances of an analyte in the biological sample, the computer-implemented process comprising:
using a computer processor having access to computer storage that stores the signal generated from processing a biological sample originating from a subject, processing the signal by:
computing, for each instance of an analyte in the biological sample, and for each window of a plurality of windows on health-condition-informative regions of the analyte, a respective value for the instance for the window based on a first function of respective marker information for the instance of the analyte for the window;
computing, for each window of the plurality of windows on the health-condition-informative region, one or more respective metrics for the window based on a second function of the respective values computed for the instances of the analyte for the window based on the first function; and
storing a data structure in memory including the one or more respective metrics computed for the window as a set of values associated with an identifier of the window of the health-condition-informative region; and
wherein the set of values corresponds to a set of features corresponding to inputs of a computational model, and
wherein the set of values for the subject for the plurality of windows represents an encoding of the signal from the biological sample of the subject.

2. A computer-implemented process for encoding a signal generated from processing a biological sample originating from a subject, the signal indicative of marker information in instances of an analyte in the biological sample, the computer-implemented process comprising:
using a computer processor having access to computer storage that stores the signal generated from processing a biological sample originating from a subject, processing the signal by:
for each window of a plurality of windows on a health-condition-informative region of an analyte:
computing, for each instance of the analyte overlapping the window, a respective value for the instance for the window based on a first function of the respective marker information for the instance overlapping the window,
computing one or more respective metrics for the window based on a second function of the respective values computed for the instances overlapping the window based on the first function, and
storing a data structure in memory including the one or more respective metrics computed for the window as a set of values associated with an identifier of the window of the health-condition-informative region; and
wherein the set of values corresponds to a set of features corresponding to inputs of a computational model, and
wherein the set of values for the subject for the plurality of windows represents an encoding of the signal generated from processing the biological sample originating from the subject.

3. A computer-implemented process for encoding methylation signals for DNA fragments from a liquid biopsy of a subject, each methylation signal indicative of methylation of CpGs in a respective DNA fragment, the process comprising:
using a computer processor having access to computer storage that stores the methylation signals for the DNA fragments from the liquid biopsy of the subject, processing the methylation signals by:
computing, for each DNA fragment, and for each window of a plurality of windows on a cancer-informative region of DNA of the subject, a respective value for the DNA fragment for the window based on a first function of the respective methylation signal for the DNA fragment for the window;
computing, for each window of the plurality of windows on the cancer-informative region, one or more respective metrics for the window based on a second function of the respective values computed for the DNA fragments for the window based on the first function; and
storing a data structure in memory including the one or more respective metrics computed for the window as a set of values associated with an identifier of the window of the cancer-informative region; and
wherein the set of values corresponds to a set of features corresponding to inputs of a computational model, and
wherein the set of values for the subject for the plurality of windows represents an encoding of the methylation signals for the DNA fragments from the liquid biopsy of the subject.

4. A computer-implemented process for encoding methylation signals for DNA fragments from a liquid biopsy of a subject, each methylation signal indicative of methylation of CpGs in a respective DNA fragment, the process comprising:
using a computer processor having access to computer storage that stores the methylation signals for the DNA fragments from the liquid biopsy of the subject, processing the methylation signals by:
for each window of a plurality of windows on a cancer-informative region of DNA of the subject:
computing, for each DNA fragment overlapping the window, a respective value for the DNA fragment for the window based on a first function of the respective methylation signal for the DNA fragment in the window,
computing one or more respective metrics for the window based on a second function of the respective values computed for the DNA fragments for the window based on the first function, and
storing a data structure in memory including the one or more respective metrics computed for the window as a set of values associated with an identifier of the window of the cancer-informative region; and
wherein the set of values corresponds to a set of features corresponding to inputs of a computational model, and
wherein the set of values for the subject for the plurality of windows represents an encoding of the methylation signals for the DNA fragments from the liquid biopsy of the subject.

5. A process for encoding methylation signals for DNA fragments from a liquid biopsy of a subject, each methylation signal indicative of methylation of CpGs in a respective DNA fragment, the process comprising:
processing the liquid biopsy of the subject to generate in computer storage a respective methylation signal for each of a plurality of DNA fragments in the liquid biopsy, the respective methylation signal indicative of methylation of CpGs in the DNA fragment;
using a computer processor having access to the computer storage that stores the methylation signals for the DNA fragments from the liquid biopsy of the subject, processing the methylations signals by:
for each window of a plurality of windows on a cancer-informative region of DNA of the subject:
computing, for each DNA fragment overlapping the window, a respective value for the DNA fragment for the window based on a first function of the respective methylation signal for the DNA fragment in the window,
computing one or more respective metrics for the window based on a second function of the respective values computed for the DNA fragments for the window based on the first function, and
storing a data structure in memory including the one or more respective metrics computed for the window as a set of values associated with an identifier of the window of the cancer-informative region; and
wherein the set of values corresponds to a set of features corresponding to inputs of a computational model, and
wherein the set of values for the subject for the plurality of windows represents an encoding of the methylation signals for the DNA fragments from the liquid biopsy of the subject.

6. A non transitory computer storage medium, comprising computer storage with data encoded thereon, the data defining a training set for training a computational model, wherein the data in the training set represents a plurality of processed samples, each processed sample originating from a respective liquid biopsy from a respective subject, wherein the data for each processed sample includes:
a respective set of values for the processed sample encoding methylation signals from DNA fragments in the processed sample, each set of values including, for each cancer-informative region of DNA, and for each window on the cancer-informative region, one or more respective metrics computed for the window and associated with an identifier of the window of the cancer-informative region, wherein each respective metric comprises a value based on computing a respective value for each DNA fragment for the window based on a first function of the respective methylation signal for the DNA fragment in the window, and a second function of the respective values computed for the DNA fragments for the window based on the first function, and
a respective label for the processed sample indicative of a respective known characteristic of the respective subject associated with the processed sample.

7. A machine comprising:
a. a processing system comprising at least one computer processor;
b. computer storage, accessible by the processing system, the computer storage comprising data defining a training set for training a computational model, data the in the training set representing a plurality of processed samples, each processed sample originating from a respective liquid biopsy from a respective subject, the data for each processed sample including:
i. a respective set of values for the processed sample encoding methylation signals from DNA fragments in the processed sample, each set of values including, for each cancer-informative region of DNA, and for each window on the cancer-informative region, one or more respective metrics computed for the window and associated with an identifier of the window of the cancer-informative region, wherein each respective metric comprises a value based on computing a respective value for each DNA fragment for the window based on a first function of the respective methylation signal for the DNA fragment in the window, and a second function of the respective values computed for the DNA fragments for the window based on the first function, and
ii. a respective label for the processed sample indicative of a respective known characteristic of the respective subject associated with the processed sample;
c. computer program code stored in the computer storage that when executed by the processing system defines a computational model having inputs for receiving a set of values for a processed sample from the training set, and having an output providing a computed characteristic based on the set of values received at the inputs and parameters of a function, the computer program code further configuring the processing system to access the training set and train the computational model by repeatedly:
i. applying the respective sets of values for processed samples in the training set to the inputs of the computational model,
ii. receiving, from the output of the computational model, respective outputs in response to the respective set of values applied to the inputs of the computational model,
iii. comparing the respective outputs for the respective sets of values to the respective labels for the processed samples corresponding to the input sets of values, and
iv. adjusting the parameters of the computational model to reduce error between the respective outputs from the computational model and the respective labels for the processed samples.

8. A cancer recognition system for recognizing a risk of presence of a neoplasm in a subject based on a liquid biopsy from the subject, comprising:
equipment having an input that receives a liquid biopsy and an output that provides a methylation signal for the liquid biopsy, the methylation signal indicative of methylation of CpGs of DNA fragments in the liquid biopsy; and
an analytical platform having an input receiving the methylation signal for the liquid biopsy from the equipment and having a processing system that, in response to computer program instructions, is configured to process the methylation signals and to:
for each window of a plurality of windows on a cancer-informative region of DNA of the subject:
compute, for each DNA fragment overlapping the window, a respective value for the DNA fragment for the window based on a first function of the respective methylation signal for the DNA fragment in the window,
compute one or more respective metrics for the window based on a second function of the respective values computed for the DNA fragments for the window based on the first function,
store a data structure in memory including the one or more respective metrics computed for the window as a set of values associated with an identifier of the window of the cancer-informative region, wherein the set of values corresponds to a set of features corresponding to inputs of a computational model, and wherein the set of values for the subject for the plurality of windows represents an encoding of the methylation signals for the DNA fragments from the liquid biopsy of the subject, and
input the computed set of values for the subject for the set of features to a trained computational model that applies a function to the computed set of values to produce an output indicative of a risk of presence of a neoplasm in the subject.

9. In any of the preceding claims, further comprising storing the respective metrics computed for each of the plurality of windows on the region in a database as data representing the biological sample or liquid biopsy sample.

10. In any of the preceding claims, further comprising applying the data representing the biological sample or liquid biopsy sample to a computational model that determines the risk of presence of the early-stage neoplasm in the subject based on the set of values for the set of features.

11. In any of claim 1, 2, 9 or 10, wherein the first function applied to an instance of an analyte comprises a count of occurrences of marker information within the instance of the analyte within the window.

12. In claim 11, wherein the second function of the respective values computed for the instances of the analyte for the window is based on a respective ratio of a count of instances of the analyte having a specific count of occurrences of marker information to a count of instances of analyte for the window.

13. In any of claims 3 through 10, wherein the first function of the methylation signal for a DNA fragment in a window comprises a count of methylated CpGs in the DNA fragment in the window.

14. In claim 13, wherein the second function of the respective values computed for DNA fragments for a window is based on a respective ratio of a count of DNA fragments having a specific count of methylated CpGs to a count of DNA fragments for the window.

15. In claim 13, wherein the second function of the respective values computed for DNA fragments for a window is based on a count of DNA fragments having a specific count of methylated CpGs.

16. In any of claim 1, 2, 9 or 10, wherein the first function applied to an instance of an analyte comprises an indication of a pattern of marker information in the instance in the window, from among a set of possible patterns.

17. In claim 16, wherein the second function of the respective values computed for the instances of the analyte for the window is based on, for each possible pattern of marker information in the window, a ratio of a count of instances of the analyte having the pattern to a count of instances of the analyte in the window.

18. In any of claims 3 to 10, wherein the first function of the methylation signal comprises an indication of a pattern of methylation of CpGs in the DNA fragment in the window.

19. In claim 18, wherein the second function of the respective values computed for DNA fragments for a window is based on, for each possible pattern of methylation in the window, a respective ratio of a count of DNA fragments having the pattern of methylation to a count of the DNA fragments.

20. In any of the preceding claims, wherein a methylation signal comprises data indicative of a respective methylation of each CpG in a sequence of CpGs of a DNA fragment.

21. In any of the preceding claims, wherein the region comprises a plurality of CpGs wherein a number N of CpGs in the region is an integer greater than or equal to 1 and less than or equal to X, a positive integer.

22. In any of the preceding claims, wherein the region comprises a plurality of CpGs wherein a number N of CpGs in the region is an integer selected from the group consisting of 1, 2, . . . , N.

23. In any of the preceding claims, wherein the first function and the second function are computed for a plurality of different window sizes for a region.

24. In any of the preceding claims, wherein the first function comprises, for a window of size W within a region, for each possible pattern of 2W patterns, a respective count for the pattern, wherein a “count” is when a read has that pattern in that window in that region.

25. In any of the preceding claims, wherein stored data includes an identifier identifying a liquid biopsy sample or a biological sample.

26. In any of the preceding claims, wherein stored data includes an identifier identifying a subject corresponding to the liquid biopsy sample or biological sample.

27. In any of the preceding claims, wherein stored data includes a plurality of sets of values corresponding to a plurality of liquid biopsy samples or biological samples from a single subject.

28. In any of the preceding claims, wherein the set of values for the subject corresponding to the set of features includes data associating the respective metric for each window for each health-condition-informative region with an identifier of the window and an identifier of the health-condition-informative region.

29. In any of the preceding claims, wherein the data is stored in a database allowing search and retrieval of the data given one or more of an identifier of a subject, an identifier of a health-condition-informative region, and identifier of a window, or an identifier of a liquid biopsy sample or biological sample.

30. In any of claims 3 to 10, wherein an average number of cell-free DNA located per cancer-informative region is sufficient to likely include one or more cell-free DNA originating from an early-stage neoplasm if present in the subject.

31. In any of the preceding claims wherein a label for a sample is selected from a group comprising a label indicative of non-cancer and a label indicative of cancer.

32. In any of the preceding claims wherein a label for a sample is selected from a group comprising a label indicative of a type of cancer.

33. In any of the preceding claims wherein a label for a sample is indicative of a health condition.

34. In any of the preceding claims, wherein processing a sample includes processing the sample to locate cell-free DNA fragments.

35. In claim 34, wherein the cell-free DNA fragments originate from health-condition-informative regions of DNA.

36. In claim 35, wherein processing each sample includes processing located cell-free DNA fragments to determine respective methylation information related to CpGs of the located cell-free DNA fragments.

37. In claim 36, wherein the sample is processed such that an average number of cell-free DNA fragments processed per cancer-informative region is sufficient to be likely to detect one or more cell-free DNA fragments per cancer-informative region originating from a present cancer.

38. In any of the preceding claims, wherein a liquid biopsy sample or biological sample comprises plasma obtained from an asymptomatic individual.

39. In any of the preceding claims, wherein each window has a specified position within a sequence of sites of interest in a health-condition informative region, and a specified size, wherein the size is specified in terms of a number of consecutive sites of interest within the analyte.

40. In any of the preceding claims, the health-condition informative regions are selected from among the genomic regions in one or more of Table I or Table II.
